(19) United States
(12) Patent Application Publication
(10) Pub. No.: US 2018/0298024 A1
(43) Pub. Date: Oct. 18, 2018

(54) CORTISTATIN ANALOGS AND USES THEREOF

(71) Applicant: President and Fellows of Harvard College, Cambridge, MA (US)

(72) Inventors: Matthew D. Shair, Cambridge, MA (US); Henry Efrem Pelish, Cambridge, MA (US)

(73) Assignee: President and Fellows of Harvard College, Cambridge, MA (US)

(21) Appl. No.: 16/016,199

(22) Filed: Jun. 22, 2018

Related U.S. Application Data
(63) Continuation of application No. PCT/US2016/068125, filed on Dec. 21, 2016.
(60) Provisional application No. 62/387,246, filed on Dec. 23, 2015.
(60) Provisional application No. 62/297,494, filed on Feb. 19, 2016.

(51) Int. Cl. C07D 493/08; C07D 493/18; A61P 35/00

(52) U.S. Cl. CPC C07D 493/08; C07B 2200/05; A61P 35/00; C07D 493/18

(57) ABSTRACT

Specific Cortistatin derivatives with advantageous properties for in vivo administration to a host, including a human, in need thereof are provided. These novel species have advantageous pharmacokinetics, low toxicity, low to moderate hERG activity, and/or other pharmacological properties which make them stand out among the class of Cortistatins as superior candidates for human administration.

### STATEMENT OF RELATED APPLICATIONS

This application is a continuation of International Application No. PCT/US2016/068125, filed in the United States Patent and Trademark Office, Receiving Office on Dec. 21, 2016, which claims the benefit of U.S. Provisional Patent Application Nos. 62/387,246, filed Dec. 23, 2015 and 62/297,494, filed Feb. 19, 2016. The entirety of these applications are hereby incorporated by reference for all purposes.

### FIELD OF THE INVENTION

The present invention provides Cortistatin analogs with pharmacological properties amendable to in vivo administration in humans for the treatment of disorders mediated by CDK8 and/or CDK19.

### BACKGROUND

The Cortistatin family represents a group of anti-angiogenic steroidal alkaloids first isolated in 2006 from the marine sponge Corticium simplex. See, e.g., Aoki, et al., JACS (2006) 128: 3148-9. The family was initially split into four compounds: Cortistatin A, Cortistatin B, Cortistatin C, and Cortistatin D, which differ in the substitution of the D ring. The initial study showed that all four compounds are potent inhibitors of human umbilical vein endothelial cells (HUVECs) proliferation with Cortistatin A showing the strongest anti-proliferative activity (IC₅₀=1.8 nM). From Aoki's first work to the present, these natural products have been the subject of study, notably in the development of total syntheses and of new synthetic biologically active analogs.

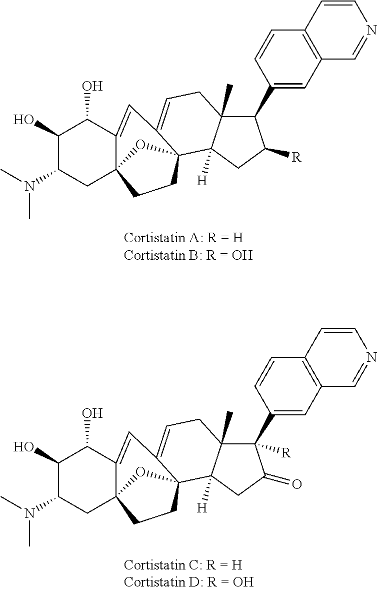

Shair et al., Nature (2015), 526: 273-276, “Mediator Kinase Inhibition Further Activates Super-Enhancer-Associated Genes in AML” describes that inhibition of CDK8 and CDK19 by Cortistatin A activates super-enhancer-associated genes in acute myeloid leukemia (AML). The activation of super-enhancer-associated genes causes an upregulation of several tumor suppressing and lineage-controlling transcription factors including CEBPA, IRF8, IRF1, and ETV6. Furthermore, leukemia cells have been shown to be sensitive to the dosage of super-enhancer-associated genes. Taken together these observations demonstrate that CDK8 and CDK19 are pharmacologically relevant targets for the treatment of AML and, by extension, other abnormal cellular proliferation acting through a like mechanism.

Cortistatin A (CA) is currently the most selective member of the naturally occurring Cortistatin family for cyclin-dependent kinase 8 (CDK8) and cyclin-dependent kinase 19 (CDK19), two kinases that coactivate the Mediator complex that is involved in the regulation of many RNA polymerase II-dependent genes. It has been shown that by inhibiting CDK8 and CDK19 using Cortistatin A, acute myeloid leukemia (AML) growth can be abated.

Baran, et al., JACS(2008), 130: 7241-7243 titled “Synthesis of (+)-Cortistatin A” describes a semisynthetic route to Cortistatin A starting from Prednisone, which already contains 70% of the desired carbon atoms and the corresponding, enantiopure chirality of Cortistatin A. The synthetic process utilized an isohypsic (oxidation-state conserving) cascade to construct the 9-(10,19)-abeo-androstane skeleton as well as a previously unreported alcohol-directed, dibromination reaction to afford Cortistatin A in approximately 3% overall yield. This synthesis, along with 3-substituted amino derivatives in the A ring, is described in U.S. Pat. No. 8,642,766 titled “Synthesis of (+) Cortistatin A and Related Compounds”. The application of Cortistatins for inhibition of retroviral replication is described in WO 2012/096934 titled “Inhibitors of Retroviral Replication”.

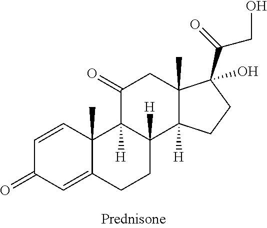

The publication by Nicolaou, et al., Angew. Chem. Int. Ed. (2008), 47: 7310-7313 titled “Total Synthesis of (+)-Cortistatin A” describes a total synthetic route to Cortistatin A starting from an enantiomerically enriched bicyclic enone (shown below) utilizing a Sonogashira coupling as well as a Suzuki-Miyaura coupling.

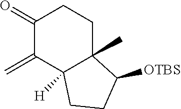 [SMILES: C=C1C(=O)CC[C@]2(C)[C@@H](O[Si](C)(C)C(C)(C)C)CC[C@@H]12]

The publication by Shair, et al., JACS (2008), 130: 16864-16866 titled “Enantioselective Synthesis of (+)-Cortistatin A, a Potent and Selective Inhibitor of Endothelial Cell Proliferation” describes the enantioselective synthesis of Cortistatin A starting from a different bicyclic enone (shown below) that was derived in two steps from the Hajos-Parrish ketone. The synthesis utilized a highly diastereoselective aza-Prins cyclization coupled with transannular etherification. The synthesis was also designed in such a way that the A, B, C, and D rings could be probed for their contribution to the antiangiogenic activity of Cortistatin A.

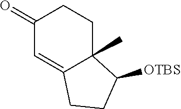 [SMILES: CC(C)(C)[Si](C)(C)O[C@H]1CCC2=CC(=O)CC[C@@]21C]

The publication by Myers et al., Nature Chemistry (2010), 2: 886-892 titled “Synthesis of Cortistatins A, J, K, and L” describes the synthesis of the A, J, K, and L members of the Cortistatin family. The synthesis features an intermediate (shown below) that can be within a few steps derivatized to either Cortistatin A, J, K, or L. The intermediate was synthesized in 9 steps and was converted to the final Cortistatins in 3 or 4 step sequences involving addition of a 7-isoquinolyl organometallic intermediate followed by deprotection.

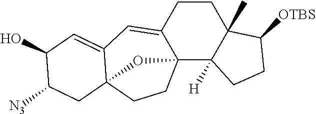 [SMILES: CC(C)(C)[Si](C)(C)O[C@H]1CC[C@@H]2[C@]1(C)CCC1=CC3=C[C@H](O)[C@@H](N=[N+]=[N-])C[C@]34CC[C@@]12O4]

U.S. Pat. No. 9,127,019 titled “Cortistatin Analogs and Synthesis Thereof” filed by Flyer, et. al., and assigned to the President and Fellows of Harvard College describes analogs of Cortistatins A, J, K, and L having the general Formula I and salts thereof, and the synthesis thereof, wherein R₁, R₂, R₃, R₄, n, and m are as described therein.

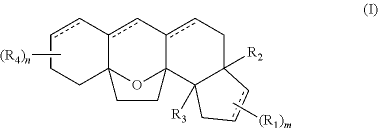

WO 2015/100420 titled “Cortistatin Analogs and Syntheses and Uses Thereof” filed by Shair, et al., and also assigned to the President and Fellows of Harvard College describes further analogs of Cortistatin and an improved modular synthesis of various sub formulas of Formula I including Formula A and Formula E shown below, wherein the variables used are defined therein.

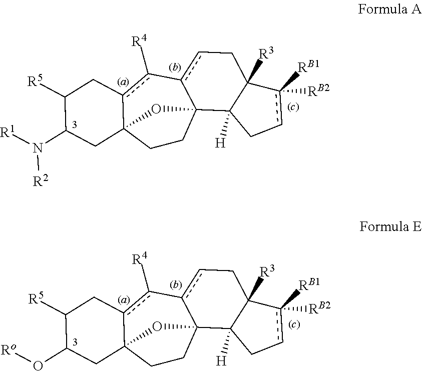

WO 2016/182904 titled “Targeted Selection of Patients for Treatment with Cortistatin Derivatives” filed by Shair, et al., and assigned to the President and Fellows of Harvard College describes the selection of patients for treatment with Cortistatin Analogues. WO 2016/182932 titled “Cortistatin Analogues, Syntheses, and Uses Thereof” filed by Shair, et al., and assigned to the President and Fellows of Harvard College describes additional Cortistatin analogues.

Other synthetic and biological descriptions of Cortistatin A and analogs of Cortistatin A have been described in Chiu et al., Chemistry (2015), 21: 14287-14291, titled “Formal Total Synthesis of (+)-Cortistatins A and J”; Valente et al., Current HIV Research (2015), 13: 64-79, titled “Didehydro-Cortistatin A Inhibits HIV-1 Tat Mediated Neuroinflammation and Prevents Potentiation of Cocaine Reward in Tat Transgenic Mice”; Motomasa et al., Chemical & Pharma. Bulletin (2013), 61: 1024-1029 titled “Synthetic Studies of Cortistatin A Analog from the CD-ring Fragment of Vitamin D2”; Valente et al., Cell Host & Microbe (2012), 12: 97-108 titled “An Analog of the Natural Steroidal Alkaloid Cortistatin A Potently Suppress Tat-dependent HIV Transcription”; Motomasa et al., ACS Med. Chem. Lett. (2012), 3: 673-677 titled “Creation of Readily Accessible and Orally Active Analog of Cortistatin A”; Danishefsky et al., Tetrahedron (2011) 67: 10249-10260 titled “Synthetic Studies Toward (+)-Cortistatin A”; Motomasa et al., Heterocycles (2011), 83: 1535-1552, titled “Synthetic Study of Carbocyclic Core of Cortistatin A, an Anti-angiogenic Steroidal Alkaloid from Marine Sponge”; Motomasa et al., Org. Lett. (2011), 13: 3514-3517, titled “Stereoselective Synthesis of Core Structure of Cortistatin A”; Baran et al., JACS (2011), 133: 8014-8027, titled “Scalable Synthesis of Cortistatin A and Related Structures”; Hirama et al., JOC (2011), 76: 2408-2425, titled “Total Synthesis of Cortistatins A and J”; Zhai et al., Org. Lett. (2010), 22: 5135-5137, titled “Concise Synthesis of the Oxapentacyclic Core of Cortistatin A”; Stoltz et al., Org. Biomol. Chem. (2010), 13: 2915-2917, titled “Efforts Toward Rapid Construction of the Cortistatin A Carbocyclic Core via Enyne-ene Metathesis”; Sarpong et al., Tetrahedron (2010), 66: 4696-4700, titled “Formal Total Synthesis of (+−)-Cortistatin A”; Nicolaou et al., Angewandte Chemie (2009), 48: 8952-8957, titled “Cortistatin A is a High-Affinity Ligand of Protein Kinases ROCK, CDK8, and CDK11”.

The publication by Hessel et al., Neurobiology of Aging (2003), 24: 427-435 titled “Cyclin C Expression is Involved in the Pathogenesis of Alzheimer's Disease” shows that Cyclin C is more highly expressed in neurons and astrocytes of Alzheimer's disease (AD) patients, and thus specific small molecule inhibition of CDK8 may also prove beneficial for treating degenerative disorders such as AD.

U.S. Patent application publication US2013/0217014 and PCT application WO2013/122609 titled “Methods of Using CDK8 Antagonists” filed by Firestein, et al., describes the use of CDK8 antagonists in general against various cancers. As described therein, part of the mediator complex CDK8 has a conserved function in transcription as described by Taatjes, D. J., Trends Biochem Sci 35, 315-322 (2010); and Conaway, R. C. and Conaway, J. W., Curr Opin Genet Dev 21, 225-230 (2011). CDK8 has also been reported as an oncogene in both colon cancer (Firestein R. et al., Nature 455:547-51 (2008); Morris E. J. et al., Nature 455:552-6 (2008); Starr T. K. et al., Science 323:1747-50 (2009)) and melanoma (Kapoor A. et al., Nature 468:1105-9 (2010)). CDK8, which is upregulated and amplified in a subset of human colon tumors, is known to transform immortalized cells and is required for colon cancer proliferation in vitro. Similarly, CDK8 has also been found to be overexpressed and essential for proliferation in melanoma. Kapoor, A. et al., Nature 468, 1105-1109 (2010). CDK8 has been shown to regulate several signaling pathways that are key regulators of both ES pluripotency and cancer. CDK8 activates the Wnt pathway by promoting expression of β-Catenin target genes (Firestein, R. et al., Nature 455, 547-551 (2008)) or by inhibiting E2F1, a potent inhibitor of β-Catenin transcriptional activity. Morris, E. J. et al., Nature 455, 552-556 (2008). CDK8 promotes Notch target gene expression by phosphorylating the Notch intracellular domain, activating Notch enhancer complexes at target genes. Fryer C. J. et al., Mol Cell 16:509-20 (2004).

Despite Cortistatin A's unique biological profile and the plethora of studies around the cores structure, it is not suitable as a potential drug due to its high toxicity and/or pharmacokinetic challenges. In fact, despite the potent nanomolar level CDK8 and CDK19 inhibitory activity of Cortistatin A and certain analogs, none have been advanced to clinical trials for the treatment of cancer or any other indication. For example, when Cortistatin A is administered to mice once-daily at a dose that fully inhibits CDK8 kinase activity in vivo, the experiment has to be terminated due to unacceptable weight loss in the animal. Furthermore, certain Cortistatin derivatives produce unacceptable hERG activity in the animal. The hERG protein, part of the potassium ion channel, contributes to the electrical activity of the heart that coordinates the heart's beating activity. When the electrical activity is compromised, it can result in a dangerous condition referred to as long QT prolongation.

One of the compounds described as a species in WO 2015/100420 (Paragraph 224 of page 91) is Compound A ((3S,3aR,9R,10aR,12aS,12bR)-3-(isoquinolin-7-yl)-3a-methyl-1,2,3,3a,4,7,8,9,10,11,12,12b-dodecahydro-10a,12a-epoxybenzo[4,5]cyclohepta[1,2-e]inden-9-ol).

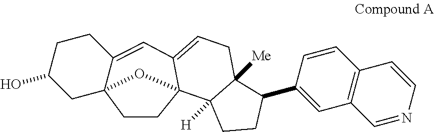

It has been discovered that Compound A is highly unusual among Cortistatin A analogs because it exhibits a combination of low hERG activity (wherein low hERG activity is defined as IC₅₀>1 μM), high selectivity against off-target enzymes and receptors and low toxicity (no significant weight loss, for example, <15% weight loss over 7 day dosing). The low toxicity results in higher tolerability of the drug, which allows for dosing at a higher level and thus better efficacy.

Given the therapeutic importance of inhibiting CDK8 and/or CDK19 in the treatment of tumors, cancer, and other disorders mediated by these enzymes, it is a goal of the invention to identify compounds that selectively inhibit CDK8 and/or CDK19 and have advantageous medicinal properties.

Thus, it is an object of this invention to provide new compounds, methods and compositions for the treatment of disorders mediated by CDK8 and CDK19, including tumors, cancers, disorders related to abnormal proliferation, inflammatory disorders, immune disorders, autoimmune disorders and other disorders that act through a similar pathway and are advantageous for human administration and therapy.

### SUMMARY

The present invention provides specific Cortistatin derivatives with advantageous properties for in vivo administration to a host, including a human, in need thereof. Specifically, these novel species have advantageous pharmacokinetics, low toxicity and/or other pharmacological properties which make them stand out among the class of Cortistatins as superior candidates for human administration. The compounds have been found to exhibit only low to moderate hERG activity and can be administered in therapeutically effective amounts without significant weight loss or unacceptable toxicity. Because of the low toxicity of these compounds, a higher Cmax and/or AUC can be achieved in a manner that allows for dosing in a range that provides high efficacy.

In particular, the invention provides Compound B, Compound C, and Compound D, shown below, or a pharmaceutically acceptable salt, prodrug, N-oxide, and/or a pharmaceutically acceptable composition thereof. Each compound has a unique substituent at the 3-position of the A-ring. Compound B has an (R)-pyrrolidin-3-amine, Compound C has an azetidin-3-amine and Compound D has a (3S,4S)-pyrrolidine-3,4-diol.

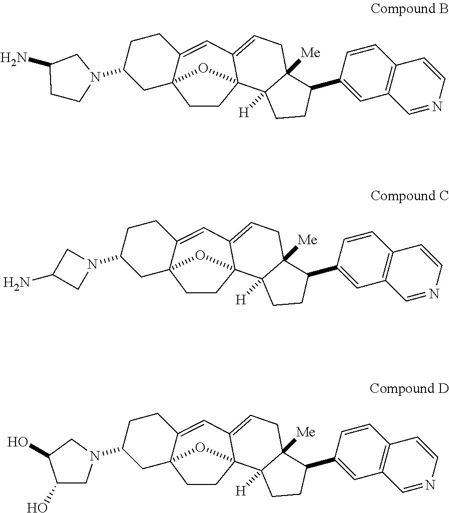

In one embodiment, a method for the treatment of a disorder mediated by CDK8 and/or CDK19, including a tumor, cancer, disorder related to abnormal proliferation, inflammatory disorder, immune disorder, or autoimmune disorder is provided that includes administering to a host in need thereof an effective amount of Compound B, C, or D or its pharmaceutically acceptable salt, prodrug, N-oxide, and/or a pharmaceutically acceptable composition thereof optionally in a pharmaceutically acceptable carrier.

Examples 3, 4, 6, 7, 12, 13, 14 provide comparative data of Compounds B, C, and D with Cortistatin A, Compound E (with unsubstituted pyrrolidine in the 3-position of the A ring) and Compound F (with unsubstituted azetidine in the 3-position of the A ring). As indicated in these examples, the compounds show superior properties. For example, Compound E has unacceptable sub-micromolar hERG activity, even though it is different from Compound B only by one amine group and from Compound D only by two hydroxyl groups. In fact, hERG activity is sometimes increased by the presence of basic groups, therefore it was truly surprising to discover that the amino substituted Compound B has less hERG activity than its unsubstituted pyrrolidine analog.

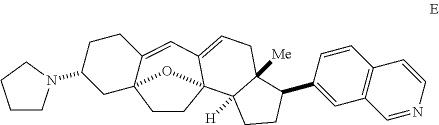

Compound F also has unacceptable sub-micromolar hERG activity.

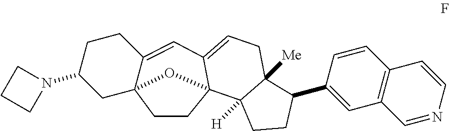

While WO 2015/100420 indicates that a hydroxyl can be in either the R- or S-configuration in the 3-position of the A-ring of Cortistatin, in fact, it was surprisingly discovered that the 3-hydroxyl group must be in the R-chirality to achieve superior properties for human administration.

Specific analogs of Compounds A, B, C, and D in the A and D rings are also provided as part of the invention. In one embodiment, a method for the treatment of a disorder mediated by CDK8 and/or CDK19, including a tumor, cancer, disorder related to abnormal proliferation, inflammatory disorder, immune disorder, or autoimmune disorder is provided that includes administering to a host in need thereof an effective amount of an analog of Compound A, B, C, or D as defined below or a pharmaceutically acceptable salt, prodrug, N-oxide, and/or a pharmaceutically acceptable composition thereof optionally in a pharmaceutically acceptable carrier.

In another embodiment, a deuterated derivative of Compound A, B, C or D or its analog is provided. Deuterium can replace one or more hydrogens in the compound. In one embodiment, deuterium is substituted for hydrogen in one or more positions in the substituent on the 3-position of the A ring. For example, in Compound A, the hydrogen in the hydroxyl can be replaced with deuterium. For example, in Compound B, a hydrogen in (R)-pyrrolidin-3-amine can be replaced with deuterium. For example, in Compound C, a hydrogen in azetidin-3-amine hydroxyl can be replaced with deuterium. For example, in Compound D, a hydrogen in (3S,4S)-pyrrolidine-3,4-diol can be replaced with deuterium. In another embodiment, deuterium is substituted for hydrogen in one or more positions in the A ring. In another embodiment, deuterium is substituted for hydrogen in one or more positions in the B ring. In another embodiment, deuterium is substituted for hydrogen in one or more positions in the C ring. In another embodiment, deuterium is substituted for hydrogen in the methyl group at the bridge carbon between the C and D rings. In another embodiment, deuterium is substituted for hydrogen in one or more positions in the D ring.

In yet another embodiment, deuterium is substituted for hydrogen in one or more positions in the isoquinoline ring.

The active compound or its pharmaceutically acceptable salt, prodrug, N-oxide, and/or a pharmaceutically acceptable composition thereof as disclosed herein is also useful for administration in combination or alternation with one or more additional pharmaceutical agents for use in combination therapy, as described in more detail herein.

The present invention thus includes at least the following features:
(i) Compounds B, C, D, and analogs of compounds A, B, C, and D as described herein;
(ii) Compounds B. C, and D, or a pharmaceutically acceptable salt, prodrug, N-oxide, and/or a pharmaceutically acceptable composition thereof, for use in treating a medical disorder which is associated with CDK8 and/or CDK19, such as a tumor, cancer, abnormal cellular proliferation, an inflammatory disorder, an immune disorder, or an autoimmune disorder;
(iii) Analogs of compounds A, B, C, and D wherein the A ring is partially unsaturated, substituted with one additional hydroxyl group, substituted with two additional hydroxyl groups, substituted with three additional hydroxyl groups, or in any combination of stereochemical configuration of the above, or a pharmaceutically acceptable salt, prodrug, N-oxide, and/or a pharmaceutically acceptable composition thereof, for use in treating a medical disorder which is associated with CDK8 and/or CDK19, such as a tumor, cancer, a disorder related to abnormal cellular proliferation, an inflammatory disorder, an immune disorder, or an autoimmune disorder;
(iv) Analogs of compounds A, B, C, and D wherein the D ring is substituted with one additional hydroxyl groups, two additional hydroxyl groups, or a cyclopropane fused ring, or a pharmaceutically acceptable salt, prodrug, N-oxide, and/or a pharmaceutically acceptable composition thereof, for use in treating a medical disorder which is associated with CDK8 and/or CDK19, such as a tumor, cancer, a disorder related to abnormal cellular proliferation, an inflammatory disorder, an immune disorder, or an autoimmune disorder;
(v) Deuterated derivatives of Compounds A, B, C, or D or their analogs or pharmaceutically acceptable salt, prodrug, N-oxide, and/or a pharmaceutically acceptable composition thereof;
(vi) Compounds B, C, and D, or a pharmaceutically acceptable salt, prodrug, N-oxide, and/or a pharmaceutically acceptable composition thereof, for use in treating a viral infection such as HIV;
(vii) Analogs of compounds A, B, C, and D wherein the A ring is partially unsaturated, substituted with one additional hydroxyl group, substituted with two additional hydroxyl groups, substituted with three additional hydroxyl groups, or in any combination of stereochemical configurations of the above, or a pharmaceutically acceptable salt, prodrug, N-oxide, and/or a pharmaceutically acceptable composition thereof, for use in treating a viral infection such as HIV;
(viii) Analogs of compounds A, B, C, and D wherein the D ring is substituted with one additional hydroxyl group, two additional hydroxyl groups, or a cyclopropane fused ring, or a pharmaceutically acceptable salt, prodrug, N-oxide, and/or a pharmaceutically acceptable composition thereof, for use in treating a viral infection such as HIV;
(ix) A process for manufacturing a medicament intended for the therapeutic use for treating or preventing a disorder listed in the methods of treatment, or generally for treating or preventing disorders mediated by CDK8 or CDK19, characterized in that a compound described above or an embodiment of the active compound is used in the manufacture;
(x) A compound described above or a salt thereof as described herein in substantially pure form (e.g., at least 90 or 95%);
(xi) A compound described above to treat a disorder described herein through a different mechanism of action; and
(xii) Methods for the manufacture of the compounds described herein.

### BRIEF DESCRIPTION OF THE FIGURES

FIG. 1 is a graph of the percent inhibition of Phosphodiesterase PDE3 activity vs concentration (μM) of Compound A (circle) or Cilostamide (square) as measured by the Panlab assay (Example 3). The x-axis is drug concentration measured in μM and the y-axis is inhibition measured as a percent. The IC₅₀ value of Compound A was 3.26 μM and the IC₅₀ value of Cilostamide was 0.059 μM.

FIG. 2 is a graph of the percent inhibition of Adenosine Transporter activity vs concentration (μM) of Compound A (circle) or Nitrobenzylthioinosine (square) as measured by the Panlab assay (Example 3). The x-axis is drug concentration measured in μM and the y-axis is inhibition measured as a percent. The IC₅₀ value of Compound A was 3.61 μM and the IC₅₀ value of nitrobenzylthioinosine was 0.35 nM. The Kᵢ value of Compound A was 1.23 μM and the Kᵢ value of nitrobenzylthioinosine was 0.12 nM. The n_{H} value of Compound A was 1.30 and the n_{H} value of nitrobenzylthioinosine was 1.10.

FIG. 3 is a graph of the percent inhibition of Dopamine Transporter activity vs concentration (μM) of Compound A (circle) or GBR-12909 (square) as measured by the Panlab assay (Example 3). The x-axis is drug concentration measured in μM and the y-axis is inhibition measured as a percent. The IC₅₀ value of Compound A was 4.90 μM and the IC₅₀ value of GBR-12909 was 0.61 nM. The Kᵢ value of Compound A was 3.89 μM and the Kᵢ value of GBR-12909 was 0.49 nM. The n_{H} value of Compound A was 0.97 and the n_{H} value of GBR-12909 was 0.77.

FIG. 4 is a graph of the percent inhibition of Tachykinin NK₁ activity vs concentration (μM) of Compound A (circle) or L-703-606 (square) as measured by the Panlab assay (Example 3). The x-axis is concentration measured in μM and the y-axis is inhibition measured as a percent. The x-axis is drug concentration measured in μM and the y-axis is inhibition measured as a percent. The IC₅₀ value of Compound A was 5.94 μM and the IC₅₀ value of L-703,606 was 3.60 nM. The Kᵢ value of Compound A was 4.30 μM and the Kᵢ value of L-703,606 was 2.60 nM. The n_{H} value of Compound A was 1.01 and the n_{H} value of L-703,606 was 0.88.

FIG. 5 is a graph of the percent inhibition of Opiate μ(OP3, MOP) activity vs concentration (μM) of Compound A (circle) or DAMIGO (square) as measured by the Panlab assay (Example 3). The x-axis is drug concentration measured in μM and the y-axis is inhibition measured as a percent. The IC₅₀ value of Compound A was 5.73 μM and the IC₅₀ value of DAMGO was 13.8 nM. The Kᵢ value of Compound A was 2.33 μM and the Kᵢ value of DAMGO was 5.61 nM. The n_{H} value of Compound A was 0.90 and the n_{H} value of DAMGO was 0.75.

FIG. 6 is a graph of the percent inhibition of the hERG ion channel vs concentration (μM) of Compound B, wherein the fitted curve indicates an IC₅₀ of approximately 11 μM (Example 4). The x-axis is drug concentration measured in μM and the y-axis is inhibition measured as a percent.

FIG. 7 is a graph of the percent inhibition of the hERG ion channel vs concentration (μM) of Compound C, wherein the fitted curve indicates an IC₅₀ of approximately 11 μM (Example 4). The x-axis is drug concentration measured in μM and the y-axis is inhibition measured as a percent.

FIG. 8 is a graph of the percent inhibition of the hERG ion channel vs concentration (μM) of Compound D, wherein the fitted curve indicates an IC₅₀ of approximately 6 μM (Example 4). The x-axis is drug concentration measured in μM and the y-axis is inhibition measured as a percent.

FIG. 9 is a graph of the percent inhibition of the hERG ion channel vs concentration (μM) of Compound E, wherein the fitted curve indicates an IC₅₀ of approximately 0.6 μM (Example 4). The x-axis is drug concentration measured in μM and the y-axis is inhibition measured as a percent.

FIG. 10 is a bar graph of light scattering unit vs concentration (μM) of Compounds A and E in the TurboSol assay. FIG. 10 approximates the solubility of Compounds A and E at the tested concentrations in the hERG assay as determined by the measured light scattering units (LSU). The x-axis is Compound E and Compound A concentration measured in μM (60% TS and 80% TS are the standards for 60% and 80% transmittance, respectively) and the y-axis is light scattering units (LSU) measured in ×1000. The 30 μM solution of Compound A was visibly cloudy at 30 μM but scattered far less light than an 80% TS standard. Compound A did not have significant LSU measurements at 3 μM and only inhibited hERG at 9% at this level (Example 5).

FIG. 11 is a bar graph of light scattering unit vs concentration (μM) of Compounds B and C in the TurboSol assay. FIG. 11 approximates the solubility of Compounds B and C at the tested concentrations in the hERG assay as determined by the measured light scattering units (LSU). The x-axis is Compound B and Compound C concentration measured in μM (60% TS and 80% TS are the standards for 60% and 80% transmittance, respectively) and the y-axis is light scattering units (LSU) measured in ×1000. The 30 μM solution of Compounds B and C were slightly above the LSU threshold. The 10 μM solutions (approximate IC₅₀ concentrations) did not have significant light scattering (Example 5).

FIG. 12 is a bar graph of light scattering unit vs concentration (μM) of Compound D in the TurboSol assay. FIG. 12 approximates the solubility of Compound D at the tested concentrations in the hERG assay as determined by the measured light scattering units (LSU). The x-axis is Compound D concentration measured in μM (60% TS and 80% TS are the standards for 60% and 80% transmittance, respectively) and the y-axis is light scattering units (LSU) measured in ×1000. The 30 μM solution of Compound D was slightly above the LSU threshold. The 10 μM solution (approximate IC₅₀ concentration) did not have significant light scattering (Example 5).

FIG. 13 is a graph of body weight (g) of mice vs time (days) during and following dosing with various concentrations of Compound A. The x-axis is time measured in days and the y-axis is weight measured in grams. As shown, only at a 10 mg/kg QD×7 dose was significant weight loss observed. The mice were treated for 7 days. Weight loss is one measure of tolerability (Example 6).

FIG. 14 is a graph of normalized body weight (%) of mice vs time (days) during and following dosing with various concentrations of Compound A. The x-axis is time measured in days and the y-axis is normalized body weight measured as a percent. As shown, only at a 10 mg/kg QD×7 dose was significant weight loss observed. The mice were treated for 7 days. Weight loss is one measure of tolerability (Example 6).

FIG. 15 is a graph of body weight (g) of mice vs time (days) during and following dosing with various concentrations of Compound D. The x-axis is time measured in days and the y-axis is weight measured in grams. As shown, only at a 10 mg/kg QD×7 dose was significant weight loss observed. The mice were treated for 7 days. Weight loss is one measure of tolerability (Example 6).

FIG. 16 is a graph of normalized body weight (%) of mice vs time (days) during and following dosing with various concentrations of Compound D. The x-axis is time measured in days and the y-axis is normalized body weight measured as a percent. As shown, only at a 10 mg/kg QD×7 dose was significant weight loss observed. The mice were treated for 7 days. Weight loss is one measure of tolerability (Example 6).

FIG. 17 is a graph of normalized body weight (%) of mice vs time (days) during and following dosing with various concentrations of Compound F. The x-axis is time measured in days and the y-axis is normalized body weight measured as a percent. The mice were treated for 7 days except for the 3 mg/kg and 10 mg/kg dosing group which received a dosing holiday after rapid weight loss. Weight loss is one measure of tolerability (Example 7).

FIG. 18 is a graph of normalized body weight (%) of MV4; 11 leukemia-bearing NSG mice vs time (days) during and following dosing with various concentrations of Cortistatin A to measure tolerability. The x-axis is time measured in days and the y-axis is normalized body weight measured as a percent. The mice were treated for the entirety of the experiment, except where noted otherwise (1.25 mg/kg, 0.0625 mg/kg, and 0.31 mg/kg due to rapid weight loss). Weight loss is one measure of tolerability (Example 7). A dose of 0.16 mg/kg IP qD was selected for a efficacy study.

FIG. 19 is a bar graph of the percent inhibitory activity of Compound A at a concentration of 1 μM against 320 different kinases. The x-axis are kinases and the y-axis is inhibition measured as a percent. As shown, only 5 kinases initially had over 50% inhibition (Example 8).

FIG. 20 is a bar graph of the percent inhibitory activity of Compound B at a concentration of 1 μM against 320 different kinases. The x-axis are kinases and the y-axis is inhibition measured as a percent. As shown, only CDK8/Cyclin C had over 50% inhibition (Example 8).

FIG. 21 is a bar graph of the percent inhibitory activity of Compound C at a concentration of 1 μM against 320 different kinases. The x-axis are kinases and the y-axis is inhibition measured as a percent. As shown, only CDK8/Cyclin C had over 50% inhibition (Example 8).

FIG. 22 is a bar graph of the percent inhibitory activity of Compound D at a concentration of 1 μM against 320 different kinases. The x-axis are kinases and the y-axis is inhibition measured as a percent. As shown, only CDK8/Cyclin C had over 50% inhibition (Example 8).

FIG. 23 is a Western blot study of Compounds A and D that shows a dose dependent response for inhibition of CDK8. Decreased staining results from inhibition of the target (Example 9).

FIG. 24 is a Western blot study of Compounds B and C that shows a dose dependent response for inhibition of CDK8. Decreased staining results from inhibition of the target (Example 9).

FIG. 25 is a graph of the ratio of CDK8 W105M mutant cells (red) to wild type cells (green) vs days of proliferation at various concentrations of Compound A. The x-axis is time measured in days and the y-axis is the ratio of CDK8 W105M cells to wild type cells. FIG. 25 illustrates the mechanism of action for Compound A by testing Compound A's antiproliferative effects against wild type AML cells (green fluorescence) and W105M CDK8 mutant cells (red fluorescence). The increase in red to green fluorescence indicates that the mutated AML cells proliferated faster than the wild type. This observation was dose dependent, supporting CDK8 as the cellular target of Compound A and the target responsible for the antileukemic activity of Compound A (Example 11).

FIG. 26 is a graph of the ratio of CDK8 W105M mutant cells (red) to wild type cells (green) vs days of proliferation at various concentrations of Compound B. The x-axis is time measured in days and the y-axis is the ratio of CDK8 W105M cells to wild type cells. FIG. 26 illustrates the mechanism of action for Compound B by testing Compound B's antiproliferative effects against wild type AML cells (green fluorescence) and W105M CDK8 mutant cells (red fluorescence). The increase in red to green fluorescence indicates that the mutated AML cells proliferated faster than the wild type. This observation was dose dependent, supporting CDK8 as the cellular target of Compound B and the target responsible for the antileukemic activity of Compound B (Example 11).

FIG. 27 is a graph of the ratio of CDK8 W105M mutant cells (red) to wild type cells (green) vs days of proliferation at various concentrations of Compound C. The x-axis is time measured in days and the y-axis is the ratio of CDK8 W105M cells to wild type cells. FIG. 27 illustrates the mechanism of action for Compound C by testing Compound C's antiproliferative effects against wild type AML cells (green fluorescence) and W105M CDK8 mutant cells (red fluorescence). The increase in red to green fluorescence indicates that the mutated AML cells proliferated faster than the wild type. This observation was dose dependent supporting CDK8 as the cellular target of Compound C and the target responsible for the antileukemic activity of Compound C (Example 11).

FIG. 28 is a graph of the ratio of CDK8 W105M mutant cells (red) to wild type cells (green) vs days of proliferation at various concentrations of Compound D. The x-axis is time measured in days and the y-axis is the ratio of CDK8 W105M cells to wild type cells. FIG. 28 illustrates the mechanism of action for Compound D by testing Compound D's antiproliferative effects against wild type AML cells (green fluorescence) and W105M CDK8 mutant cells (red fluorescence). The increase in red to green fluorescence indicates that the mutated AML cells proliferated faster than the wild type. this observation was dose dependent, supporting CDK8 as the cellular target of Compound A and the target responsible for the antileukemic activity of Compound A (Example 11).

FIG. 29 is a graph of the bioluminescence (% of original) vs number of treatment days at various doses and methods of dosing Compound A. The x-axis is time measured in days and the y-axis is bioluminescence measured as a percent of the original. In the assay, increased bioluminescence signals an increase in AML cell proliferation, and therefore the graph depicts the in vivo efficacy of Compound A, wherein lower bioluminescence represents higher efficacy (Example 13).

FIG. 30 is a graph of the log(2) scale bioluminescence (% of original) vs number of treatment days for Compounds A, F, and Cortistatin A. The x-axis is time measured in days and the y-axis is bioluminescence measured as a percent of the original. In the assay, increased bioluminescence signals an increase in AML cell proliferation, and therefore the graph depicts the in vivo efficacy of Compounds A, F, and Cortistatin A wherein lower bioluminescence represents higher efficacy. All three compounds were dosed at their highest tolerable dose (Example 13).

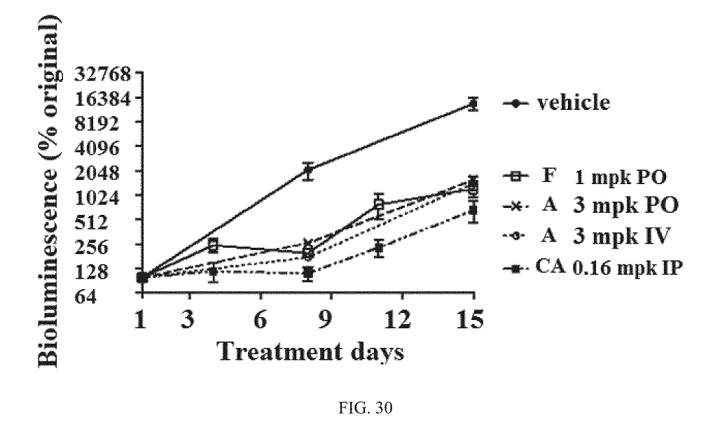

FIG. 31 is a graph of the log(2) scale bioluminescence (% of original) vs number of treatment days for Compounds B, C, D, F and Cortistatin A. The x-axis is time measured in days and the y-axis is bioluminescence measured as a percent of the original. In the assay, increased bioluminescence signals an increase in AML cell proliferation, and therefore the graph depicts the in vivo efficacy of Compounds B, C, D, F and Cortistatin A wherein lower bioluminescence represents higher efficacy. All five compounds were dosed at their highest tolerable dose (Example 13).

### DETAILED DESCRIPTION

### I. Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The present invention includes compounds with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C, ¹⁴C, ¹⁵N, ¹⁸F, ³¹P, ³²P, ³⁵S, ³⁶Cl, and ¹²⁵I respectively. In one embodiment, isotopically labelled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively or in addition, isotopes of carbon, e.g., ¹³C and ¹⁴C, may be used. In one embodiment, the isotopic substitution is deuterium for hydrogen at one or more locations on the molecule to improve the performance of the drug, for example, the pharmacodynamics, pharmacokinetics, biodistribution, half-life, stability, AUC, Tₘₐ, Cₘₐₓ, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 90%, 95% or 99% or more enriched in an isotope at any location of interest. In one embodiment, deuterium is 90%, 95% or 99% enriched at a desired location. Unless otherwise stated, the enrichment at any point is above natural abundance and enough to alter a detectable property of the drug in a human.

The compound of the present invention may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

“Pharmaceutical compositions” are compositions comprising at least one active agent, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed compound in which the parent compound is modified by making inorganic and organic, non-toxic, acid or base addition salts thereof. Generally, such salts can be prepared by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active compound is provided.

A “pharmaceutically acceptable excipient” means an excipient that is useful in preparing a pharmaceutical composition/combination that is generally safe, non-toxic and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein, including but not limited to by modulation of CDK8 and/or CDK19. Typically the host is a human. A “patient” or “host” or “subject” also refers to for example, a mammal, primate (e.g., human), cows, sheep, goat, horse, dog, cat, rabbit, rat, mice, fish, bird, chicken, and the like.

A “prodrug” as used herein, means a compound which when administered to a host in vivo is converted into a parent drug. As used herein, the term “parent drug” means any of the presently described chemical compounds described herein. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Non-limiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

A “therapeutically effective amount” of a pharmaceutical composition/combination of this invention means an amount effective, when administered to a host, to provide a therapeutic benefit such as an amelioration of symptoms or reduction or diminution of the disease itself. In non-limiting one embodiment, a therapeutically effective amount is an amount sufficient to prevent a significant increase or will significantly reduce the detectable level of cancer in the patient's blood, serum, or tissues.

### II. Active Compounds

The present invention also includes Compound B, Compound C and Compound D or a pharmaceutically acceptable salt, N-oxide, deuterated derivative, prodrug, and/or a pharmaceutically acceptable composition thereof:

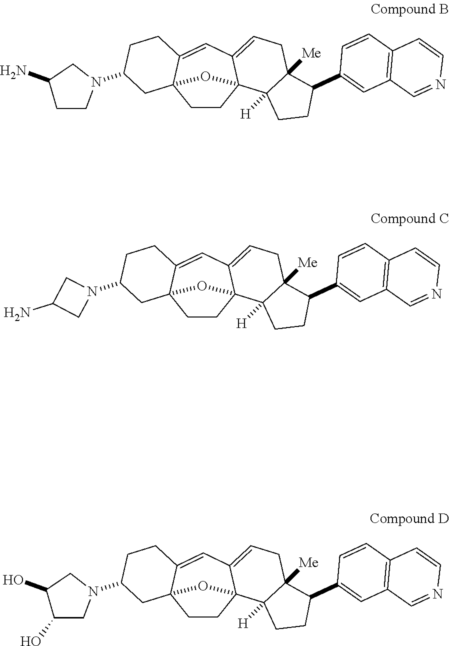 [SMILES: C[C@]12CC=C3C=C4CC[C@@H](N5CC[C@@H](N)C5)C[C@]45CC[C@]3(O5)[C@@H]1CC[C@@H]2c1ccc2ccncc2c1 ; C[C@]12CC=C3C=C4CC[C@@H](N5CC(N)C5)C[C@]45CC[C@]3(O5)[C@@H]1CC[C@@H]2c1ccc2ccncc2c1 ; C[C@]12CC=C3C=C4CC[C@@H](N5C[C@H](O)[C@@H](O)C5)C[C@]45CC[C@]3(O5)[C@@H]1CC[C@@H]2c1ccc2ccncc2c1]

The present invention also includes the following analogs of Compound A or a pharmaceutically acceptable salt, N-oxide, deuterated derivative, prodrug, and/or a pharmaceutically acceptable composition thereof, as well as deuterated derivatives of Compound A:

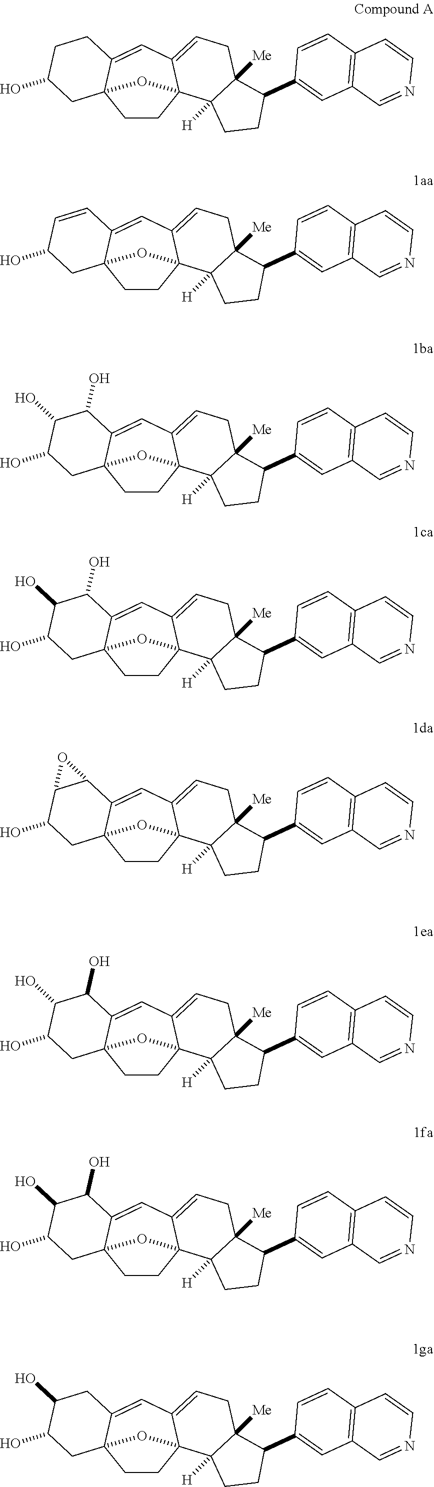

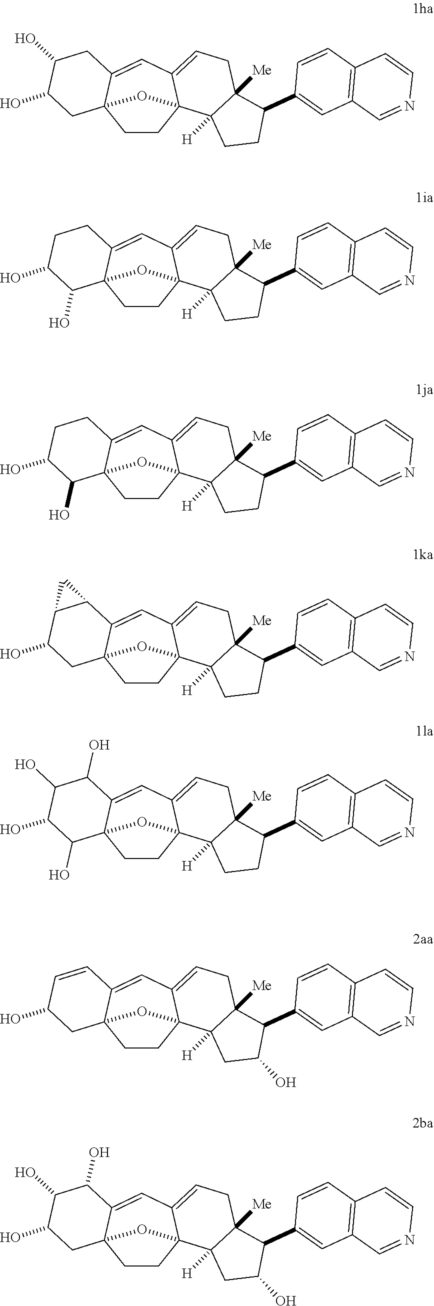

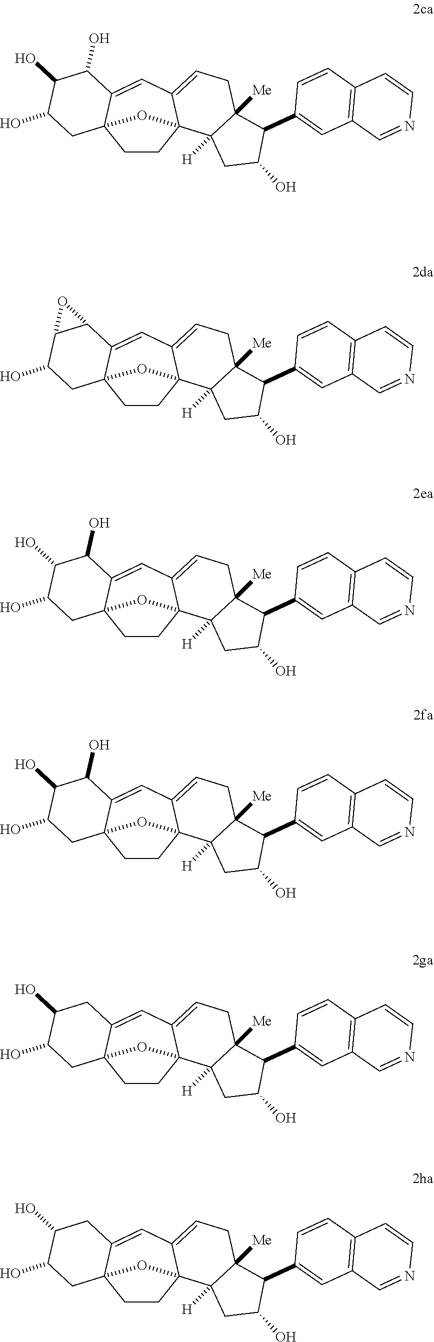

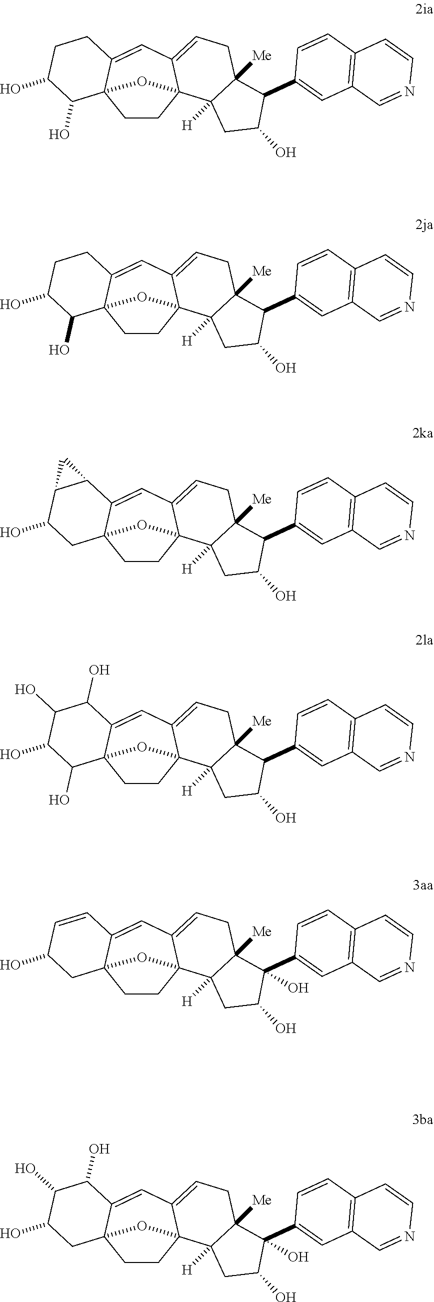

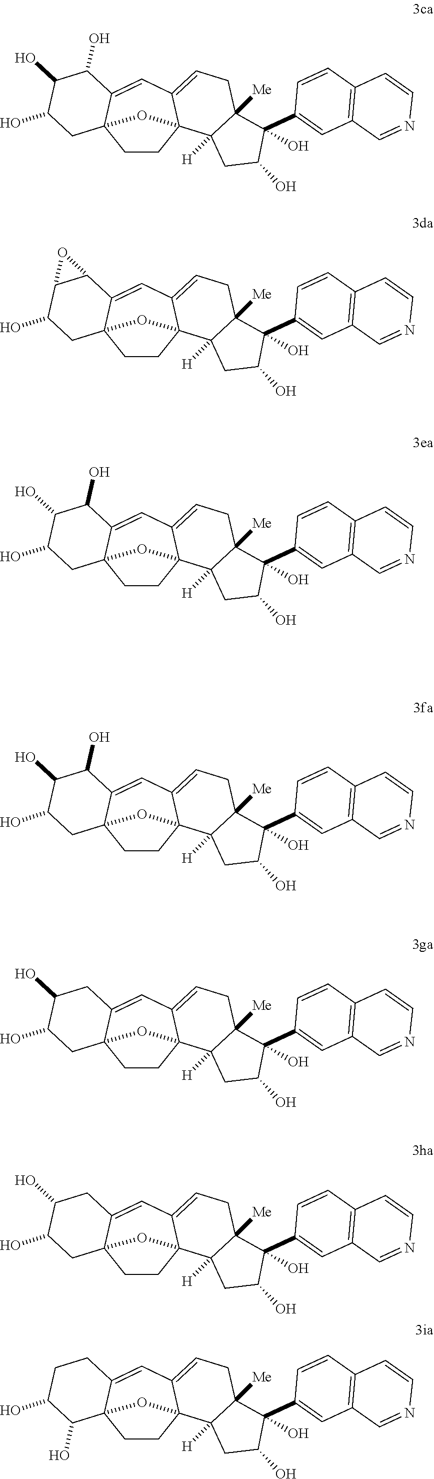

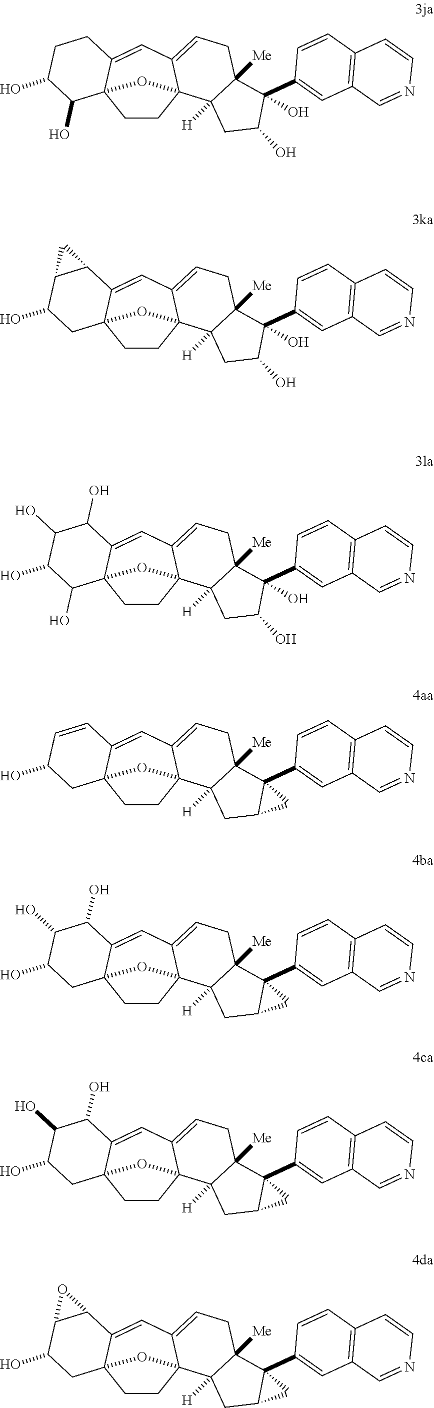

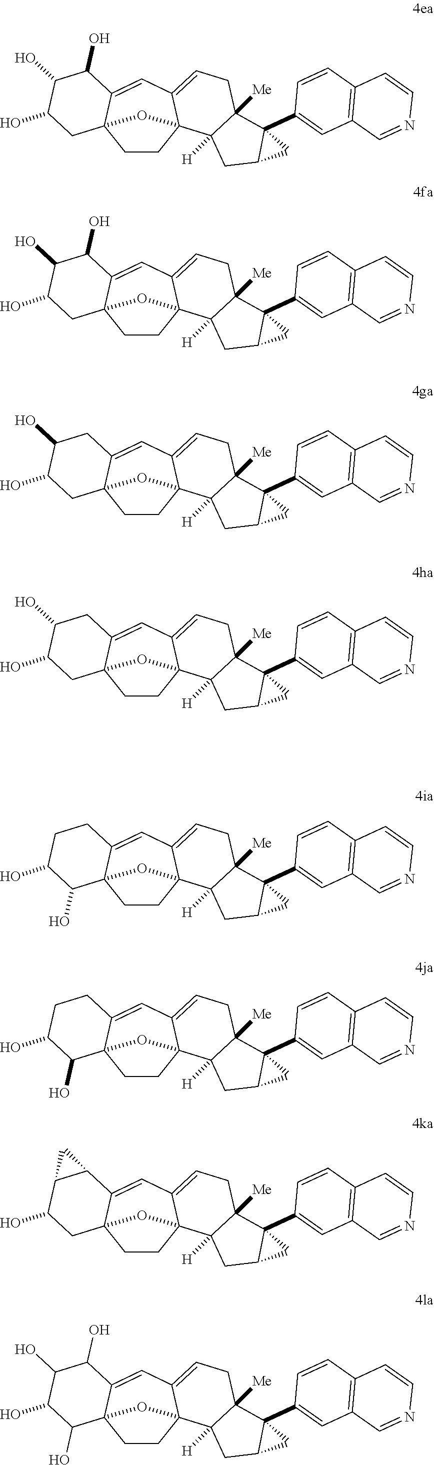

The present invention also includes the following analogs of Compound B or a pharmaceutically acceptable salt, N-oxide, deuterated derivative, prodrug, and/or a pharmaceutically acceptable composition thereof:

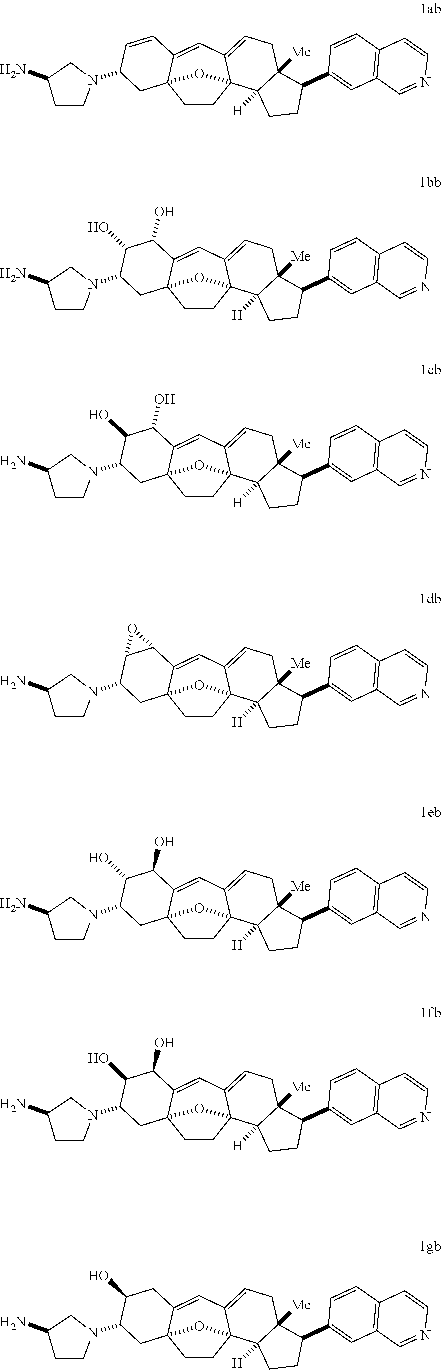

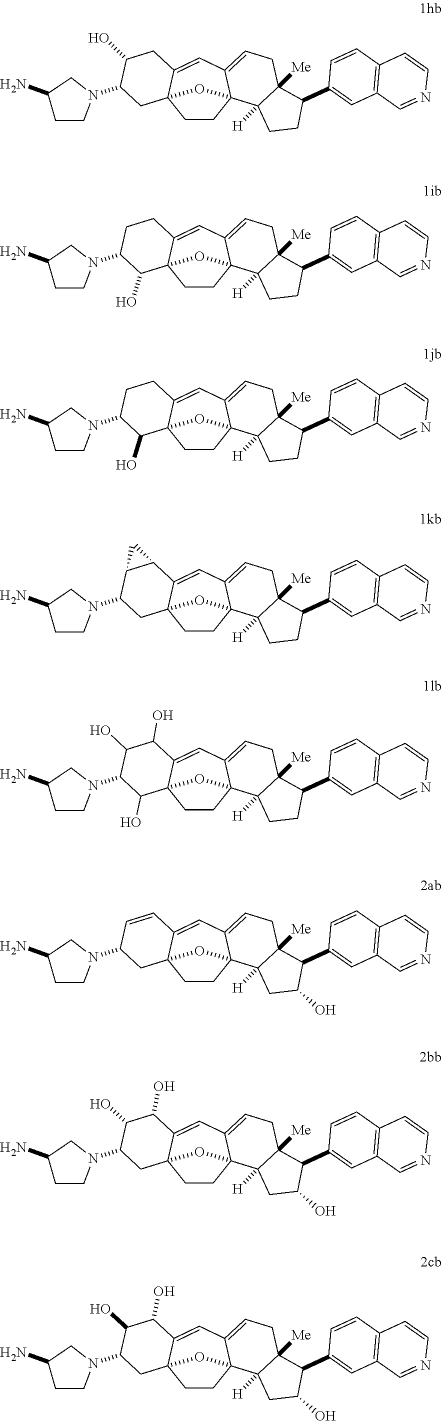

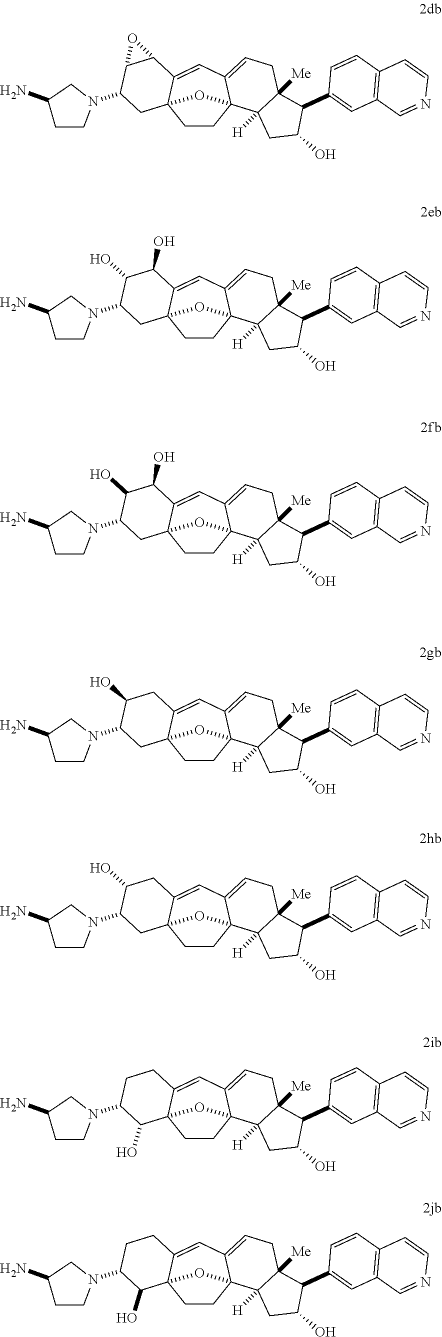

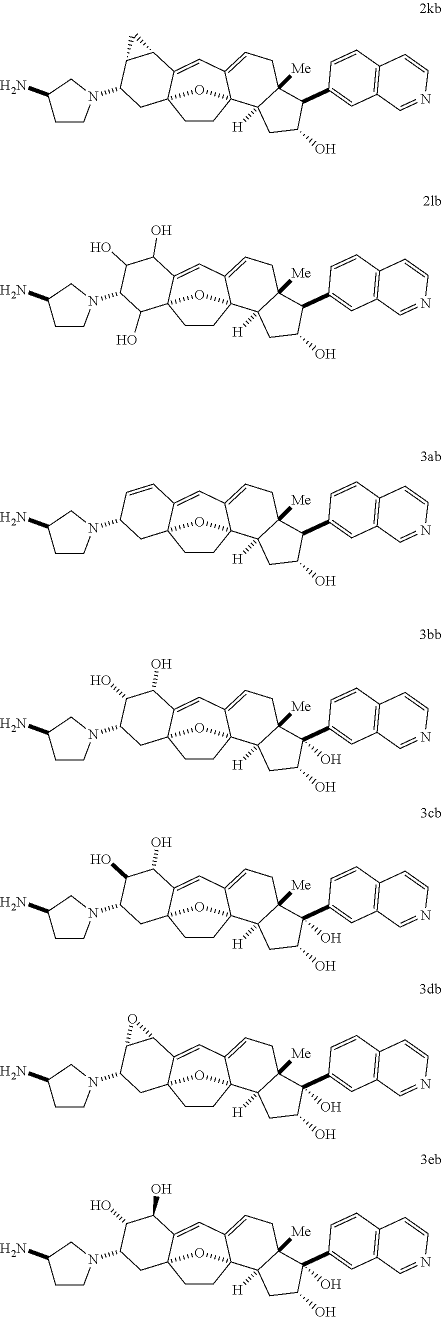

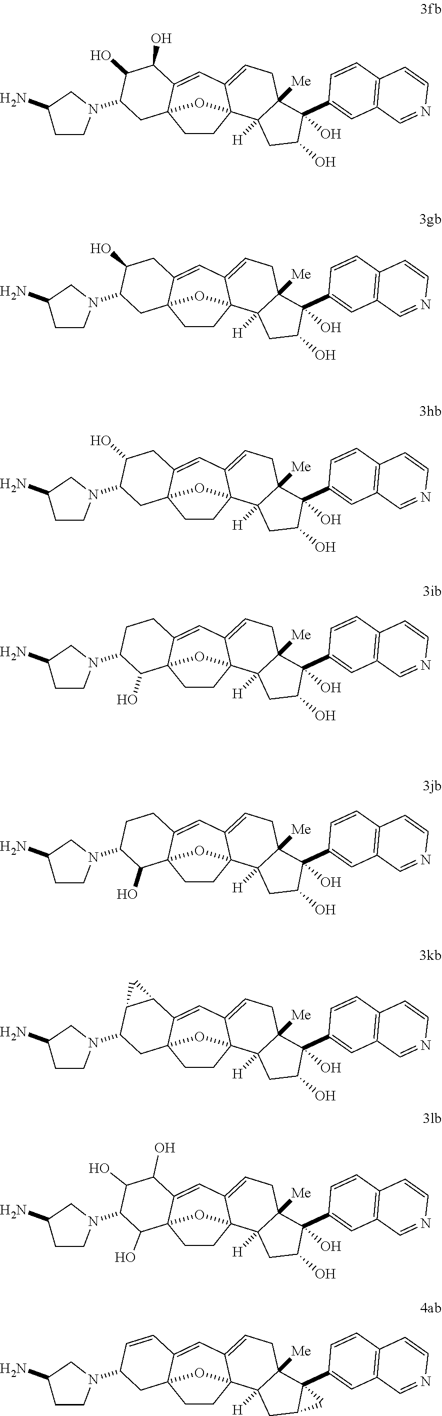

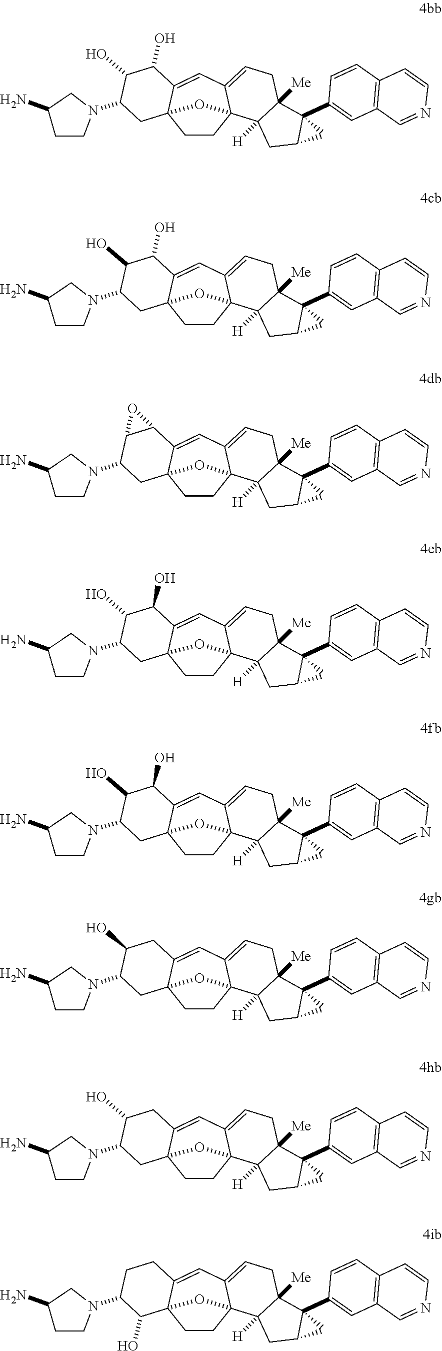

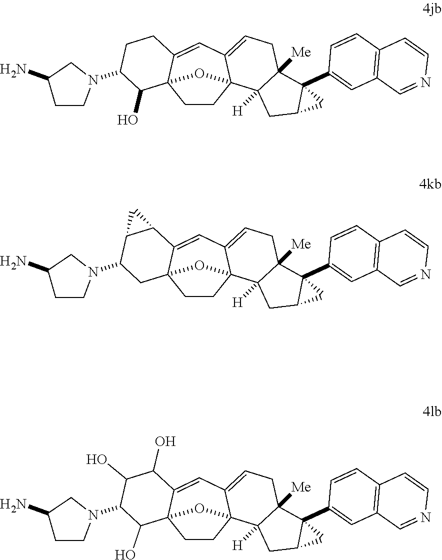

The present invention also includes the following analogs of Compound C or a pharmaceutically acceptable salt, N-oxide, deuterated derivative, prodrug, and/or a pharmaceutically acceptable composition thereof:

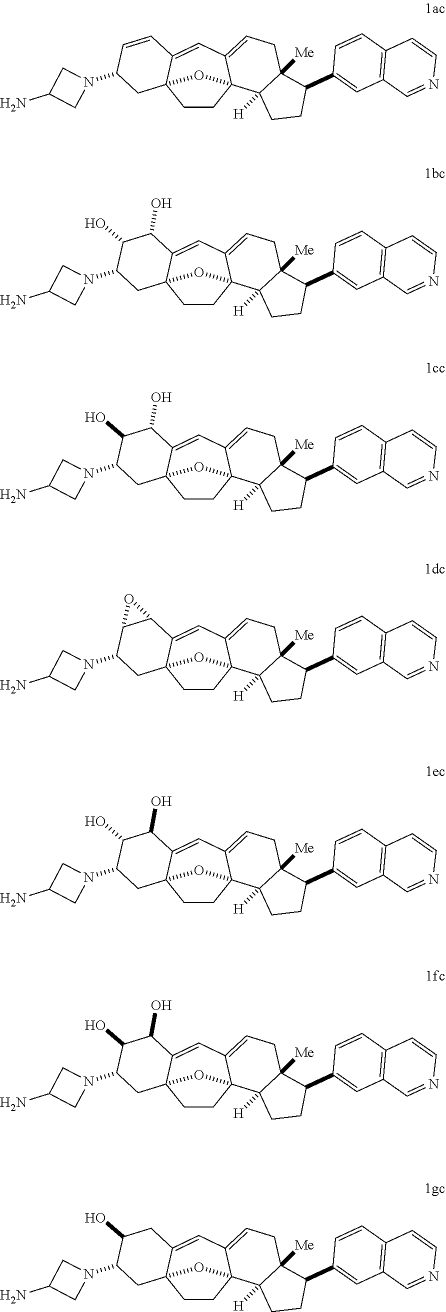

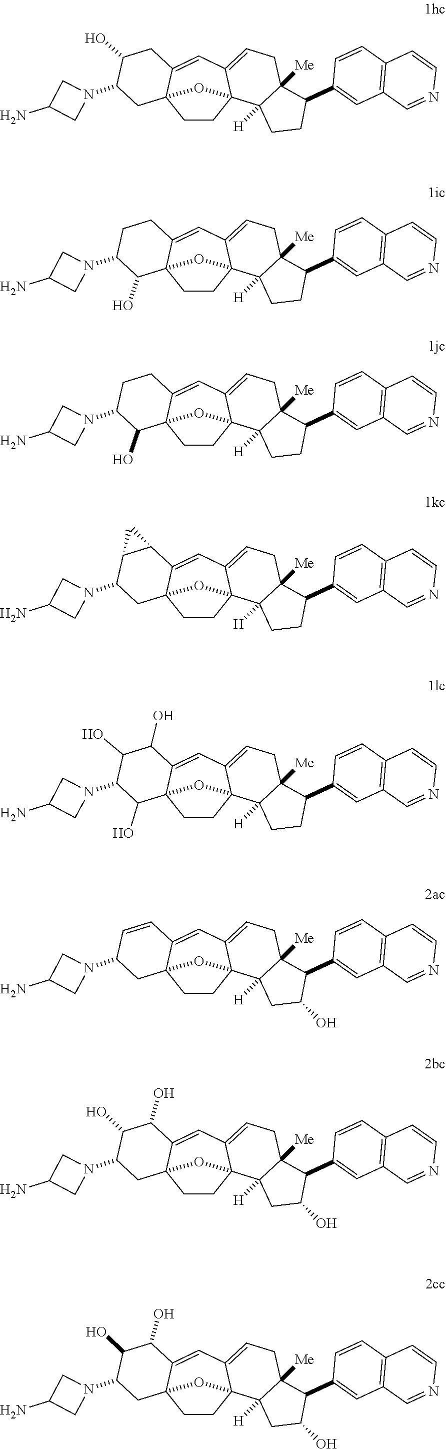

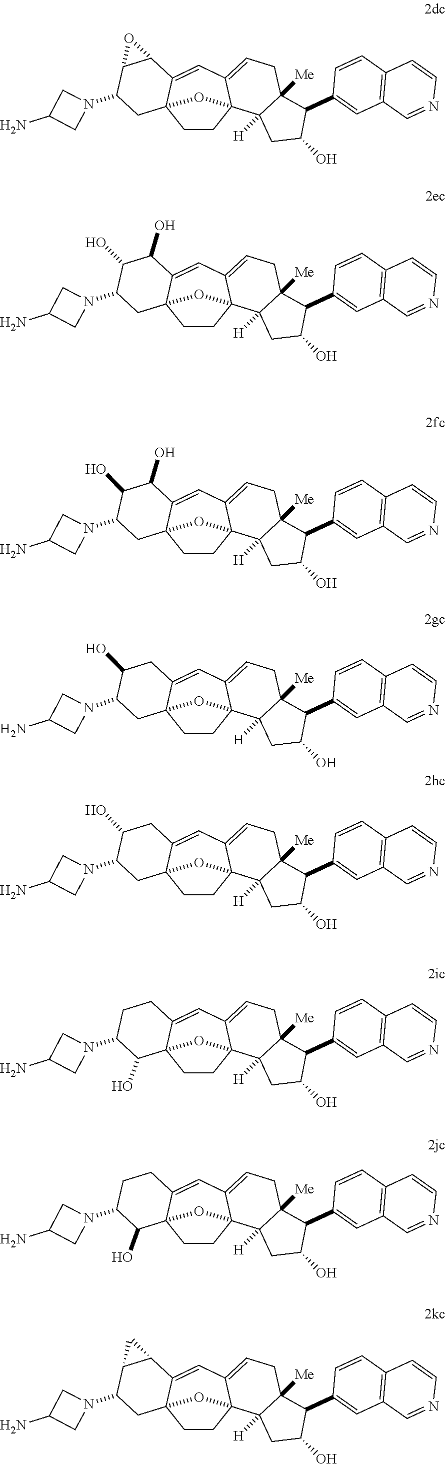

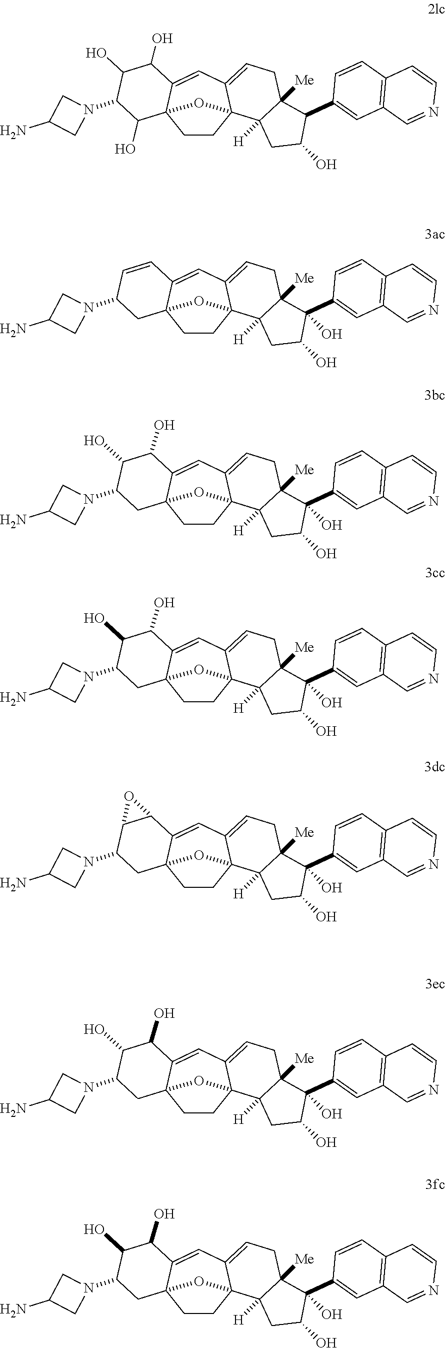

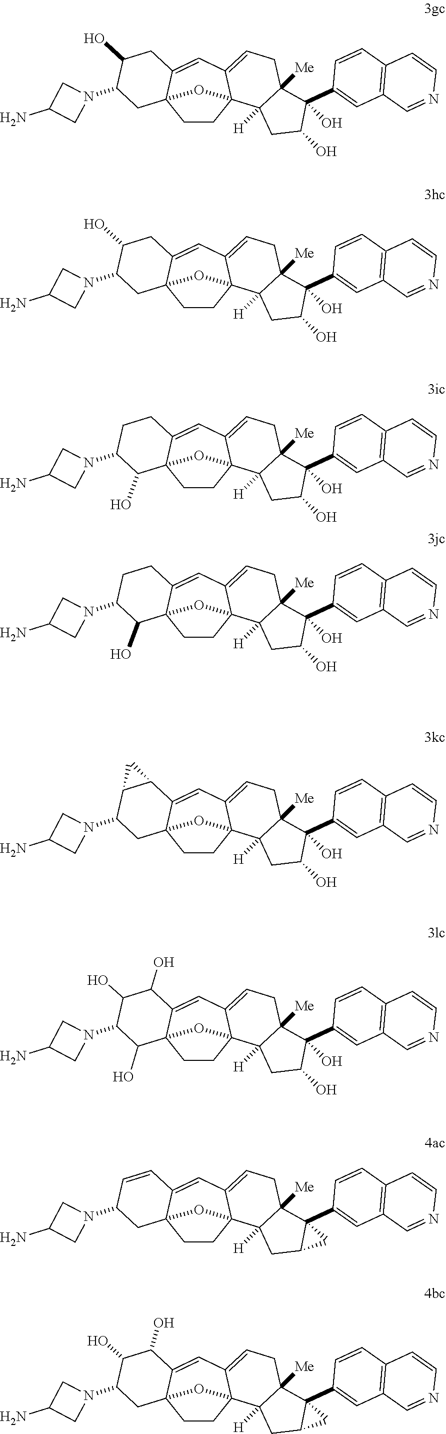

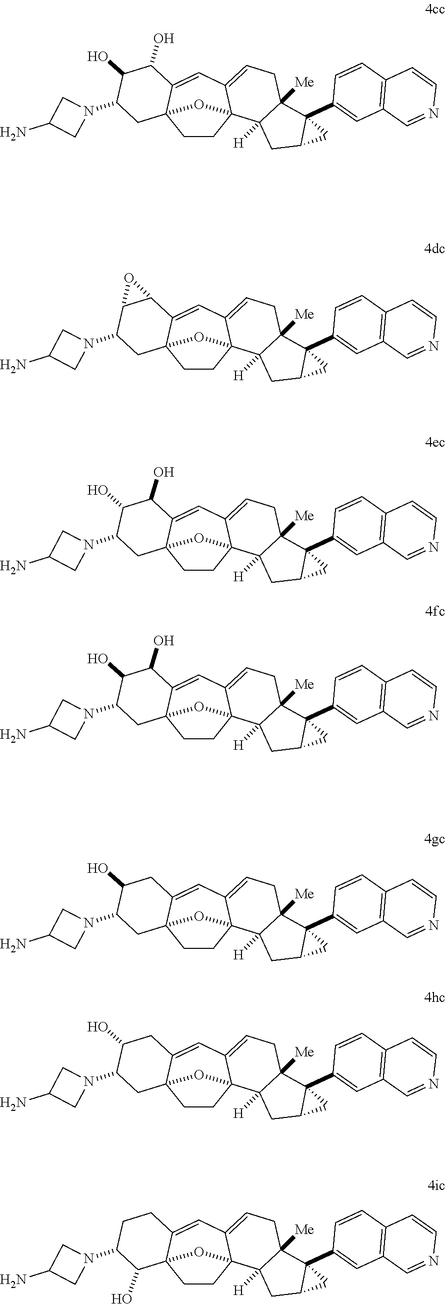

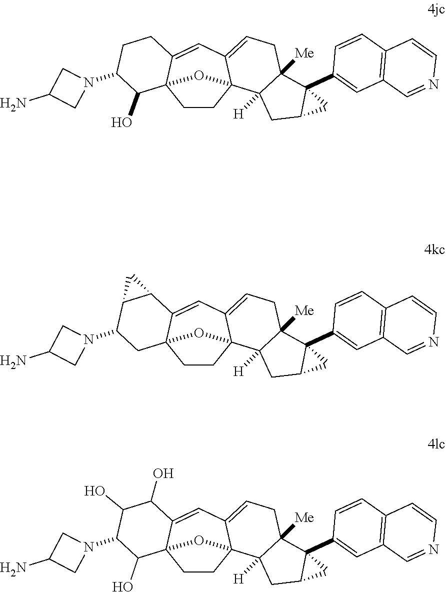

The present invention also includes the following analogs of Compound D or a pharmaceutically acceptable salt, N-oxide, deuterated derivative, prodrug, and/or a pharmaceutically acceptable composition thereof:

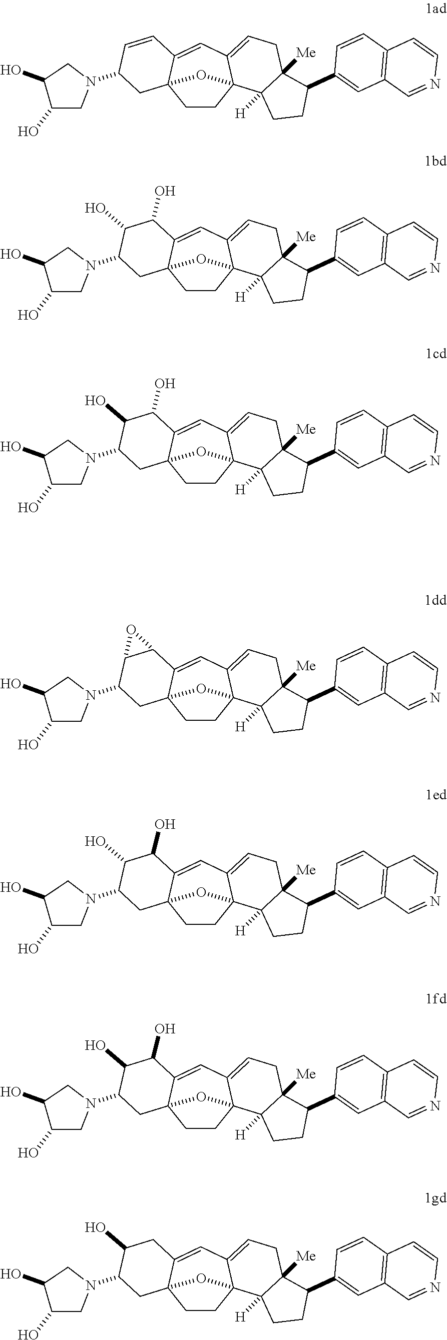

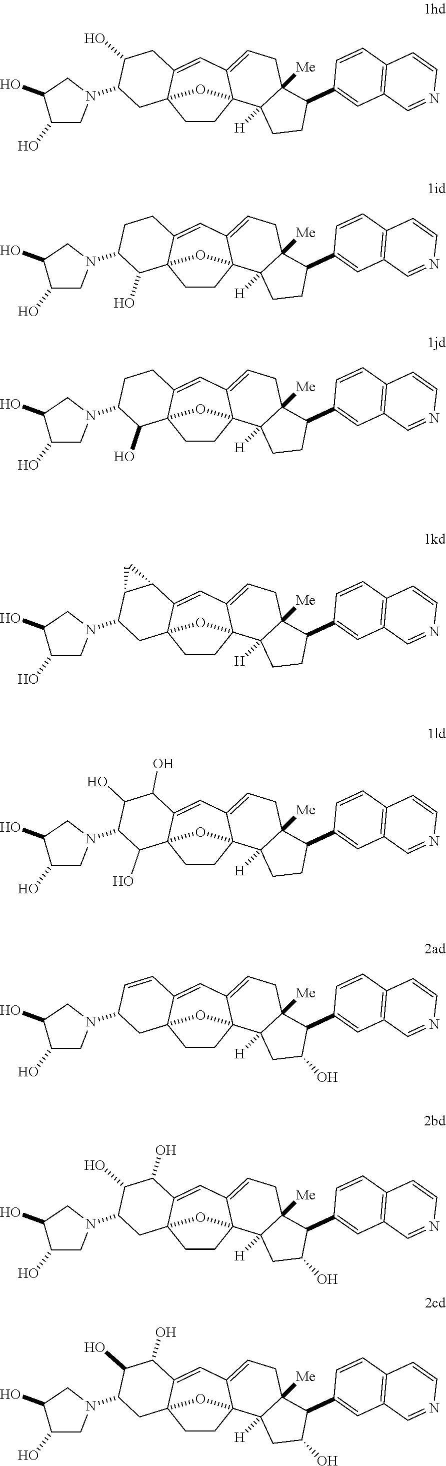

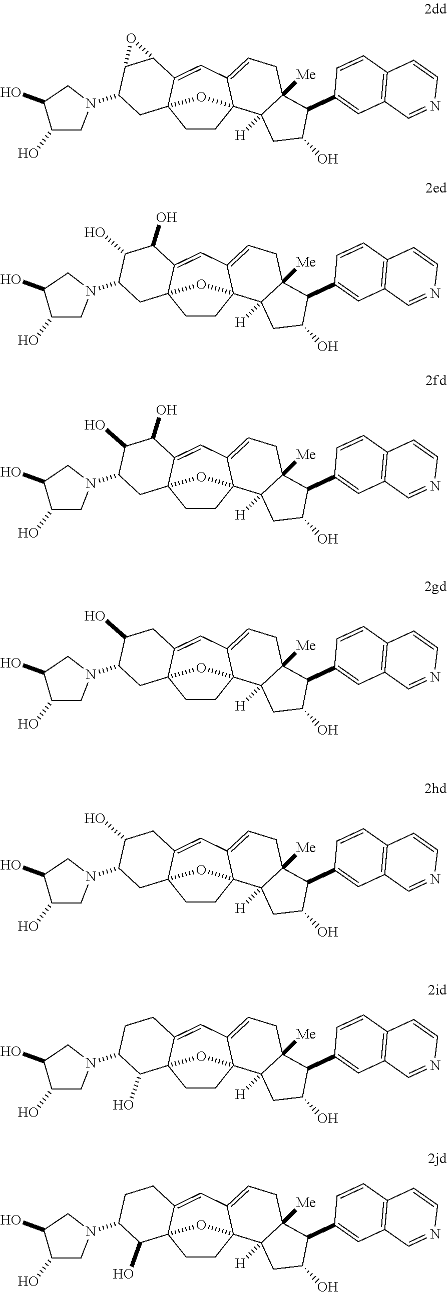

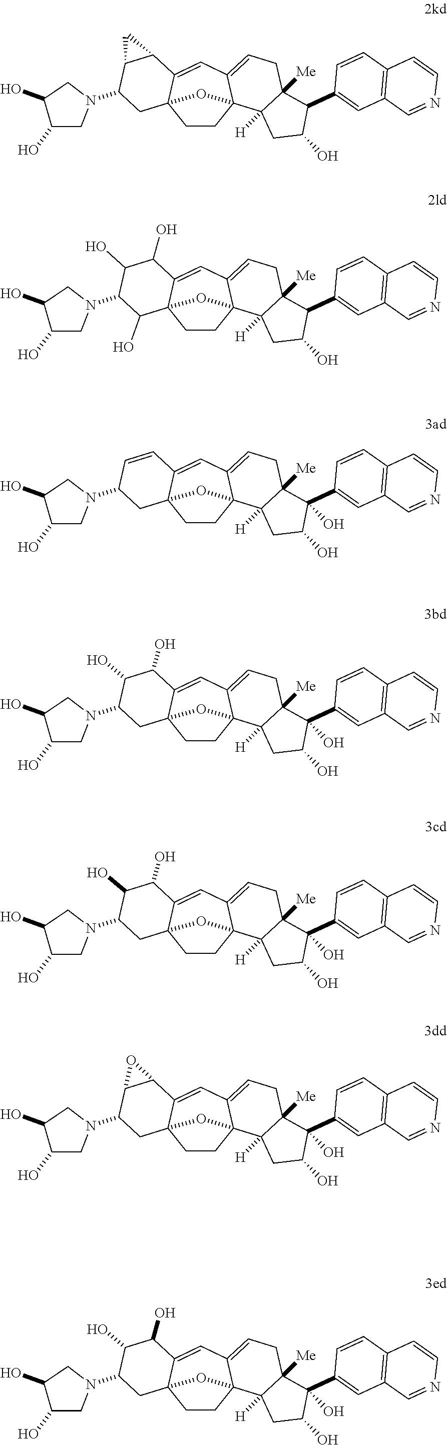

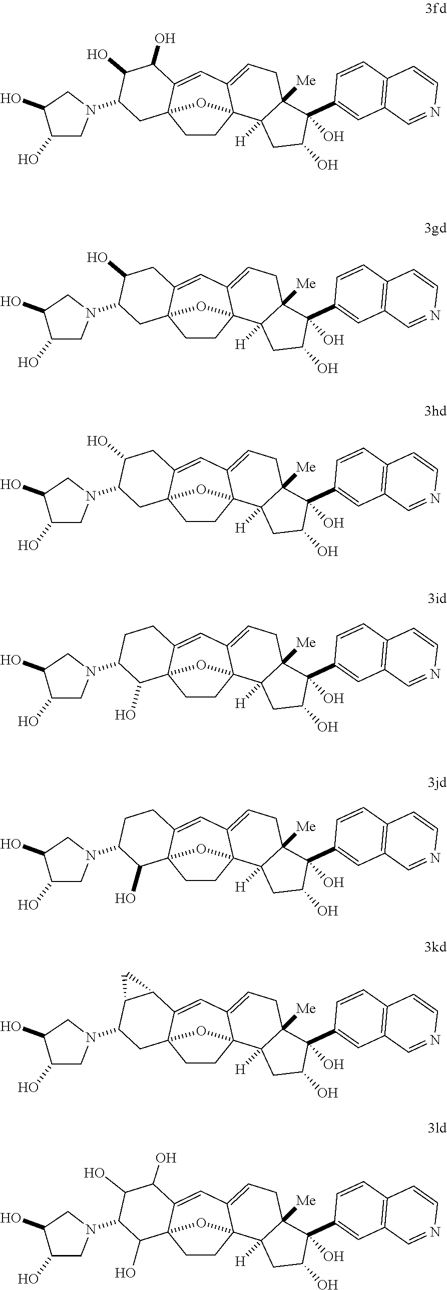

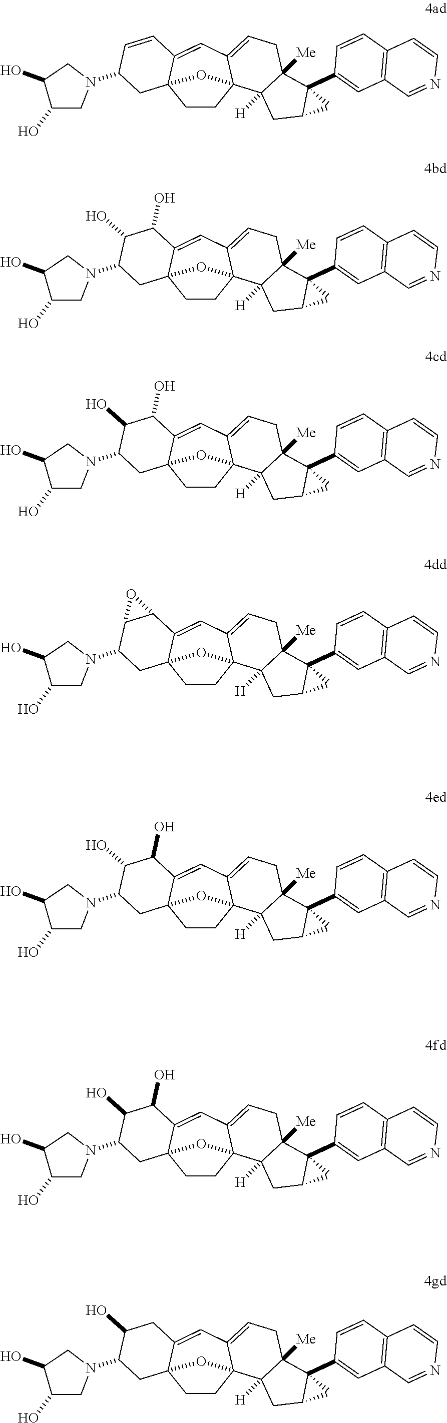

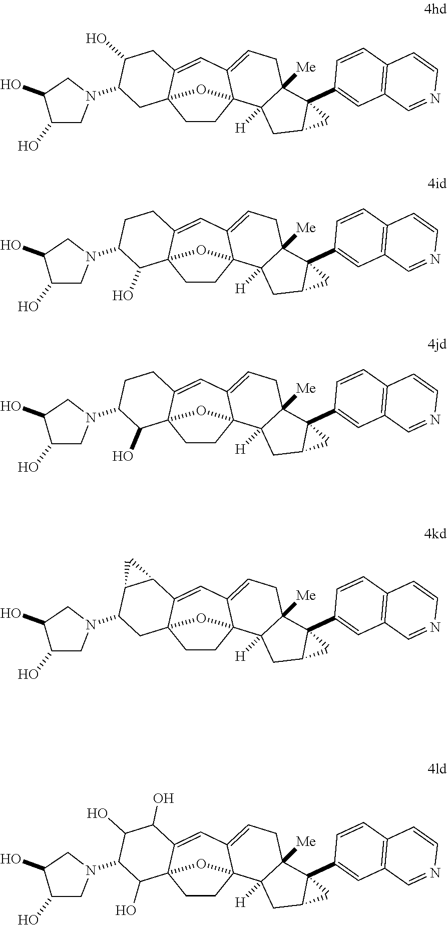

### III. Pharmaceutical Compositions

In certain embodiments, the present invention provides pharmaceutical compositions comprising a compound of the present invention or a pharmaceutically acceptable composition, salt, isotopic analog such as a deuterated derivative, or prodrug thereof, and a pharmaceutically acceptable excipient. In certain embodiments, the compound is present in an effective amount, e.g., a therapeutically effective amount or a prophylactically effective amount.

Pharmaceutically acceptable excipients include solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

Pharmaceutical compositions described herein can be prepared by any method known in the art of pharmacology. In general, such preparatory methods include the steps of bringing a compound of the present invention or a pharmaceutically acceptable composition, salt, isotopic analog, or prodrug thereof (the “active ingredient”) into association with the excipient and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable carrier, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include pharmaceutically acceptable emulsions, microemulsions, solutions, suspensions, syrups and elixirs. In addition to the active ingredient(s), the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, polymer conjugates (e.g., IT-101/CLRX101), and combinations thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the active ingredient, it is often desirable to slow the absorption of the active ingredient from subcutaneous or intramuscular injection. This can be accomplished by the use of a liquid suspension of crystalline or amorphous material with poor water solubility. The rate of absorption of the active ingredient then depends upon its rate of dissolution which, in turn, may depend upon crystal size and crystalline form. Alternatively, delayed absorption of a parenterally administered form is accomplished by dissolving or suspending the active ingredient in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient(s) can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as can be required. Additionally, the present invention contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649.912; 5,569.189; 5,704.911; 5,383.851; 5,893.397; 5,466.220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

The formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100%, (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other ophthalmically administrable formulations which are useful include those which comprise the active ingredient in microcrystalline form and/or in a liposomal preparation. Ear drops and/or eye drops are contemplated as being within the scope of this invention.

Although the descriptions of pharmaceutical compositions provided herein are principally directed to pharmaceutical compositions which are suitable for administration to humans, it will be understood by the skilled artisan that such compositions are generally suitable for administration to non-human animals. Modification of pharmaceutical compositions suitable for administration to humans to render the compositions suitable for administration to animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21′ ed., Lippincott Williams & Wilkins, 2005.

Still further encompassed by the invention are pharmaceutical packs and/or kits. Pharmaceutical packs and/or kits provided may comprise a provided composition and a container (e.g., a vial, ampoule, bottle, syringe, and/or dispenser package, or other suitable container). In some embodiments, provided kits may optionally further include a second container comprising a suitable aqueous carrier for dilution or suspension of the provided composition for preparation of administration to a subject. In some embodiments, contents of provided formulation container and solvent container combine to form at least one unit dosage form.

Optionally, a single container may comprise one or more compartments for containing a provided composition, and/or appropriate aqueous carrier for suspension or dilution. In some embodiments, a single container can be appropriate for modification such that the container may receive a physical modification so as to allow combination of compartments and/or components of individual compartments. For example, a foil or plastic bag may comprise two or more compartments separated by a perforated seal which can be broken so as to allow combination of contents of two individual compartments once the signal to break the seal is generated. A pharmaceutical pack or kit may thus comprise such multi-compartment containers including a provided composition and appropriate solvent and/or appropriate aqueous carrier for suspension.

Optionally, instructions for use are additionally provided in such kits of the invention. Such instructions may provide, generally, for example, instructions for dosage and administration. In other embodiments, instructions may further provide additional detail relating to specialized instructions for particular containers and/or systems for administration. Still further, instructions may provide specialized instructions for use in conjunction and/or in combination with additional therapy.

### IV. Methods of Treatment

In one aspect, a method of treating a disorder mediated by CDK8 and/or CDK19 kinase activity in a host, including a human, is provided comprising administering an effective amount of a compound or its pharmaceutically acceptable salt, N-oxide, deuterated derivative, prodrug, and/or a pharmaceutically acceptable composition thereof as described herein optionally in a pharmaceutically acceptable carrier. Non-limiting examples of disorders mediated by CDK8 and CDK19, include tumors, cancers, disorders related to abnormal cellular proliferation, inflammatory disorders, immune disorders, and autoimmune disorders.

In another aspect, a method of treating a disorder that is not mediated by CDK8 and/or CDK19 kinase activity in a host, but is nonetheless mediated by one or more of the compounds described herein or their pharmaceutically acceptable salts, including a human, is provided comprising administering an effective amount of a compound or its pharmaceutically acceptable salt, N-oxide, deuterated derivative, prodrug, and/or a pharmaceutically acceptable composition thereof, as described herein optionally in a pharmaceutically acceptable carrier.

In certain embodiments, the method is an in vitro method. In certain embodiments, the method is an in vivo method. In another aspect, a method of treating a condition associated with CDK8 and/or CDK19 kinase activity is provided, comprising administering to a subject in need thereof a compound of the present invention or a pharmaceutically acceptable composition, salt, isotopic analog such as a deuterated derivative, or prodrug thereof.

In certain embodiments, the condition associated with CDK8 and/or CDK19 kinase activity is a disorder related to abnormal cellular proliferation.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis.

Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

In certain embodiments, the condition associated with CDK8 and/or CDK19 kinase activity is a diabetic condition.

In certain embodiments, the condition associated with CDK8 and/or CDK19 kinase activity is a viral disease.

Human host proteins, including transcriptional cyclin-dependent kinases (CDKs), are known to contribute to the replication of several viruses, including herpes simplex virus (HSV), human immunodeficiency virus (HIV) and human cytomegalovirus (HCMV). CDK8 activity plays a role in interferon response, which is also important in cancer cell survival. Treatment with Cortistatin A increases expression of genes in MOLM-14 AML cells that have been identified as interferon gamma signaling genes and interferon responsive genes. Viruses such as HIV block interferon induction to allow more effective replication. Further, Cortistatin A has been shown to inhibit the HIV virus as well as the HIV viral protein TAT-1.

In certain embodiments, the condition associated with CDK8 and/or CDK19 kinase activity is an infection. In certain embodiments, the infection is a bacterial infection. In certain embodiments, the infection is a fungal infection. In certain embodiments, the infection is a protozoal infection. In certain embodiments, the infection is a viral infection. In certain embodiments, the viral infection is a retroviral infection, and the virus is a retrovirus, i.e., of the family Retroviridae. In certain embodiments, the viral infection is a retroviral infection, and the virus is of the family Retroviridae and subfamily Orthoretrovirinae, Alpharetrovirus, Betaretrovirus, Deltaretrovirus, Epsilonretrovirus, Gammaretrovirus, or Lentivirus. In certain embodiments, the viral infection is a retroviral infection, and the virus is of the family Retroviridae and subfamily Lentivirus. Exemplary virus of the subfamily Lentivirus include human immunodeficiency virus (HIV), simian immunodeficiency virus (SIV), feline immunodeficiency virus (FIV), equine infectious anemia virus (EIAV), and Visna virus are all examples of lentiviruses. In certain embodiments, the viral infection is a human immunodeficiency virus (HIV) infection. Other viral infections contemplated are infections with the herpes simplex virus (HSV), human immunodeficiency virus (HIV) or human cytomegalovirus (HCMV). In certain embodiments, the virus is an oncovirus, i.e., a virus which is associated with oncogenesis and/or causes cancer. In certain embodiments, treatment of the viral infection is associated with inhibition of CDK8 and/or CDK19 kinase activity.

In certain embodiments a compound of the present invention and its pharmaceutically acceptable derivatives or salts or pharmaceutically acceptable formulations containing these compounds are useful in the prevention and treatment of HIV infections and other related conditions such as AIDS-related complex (ARC), persistent generalized lymphadenopathy (PGL), AIDS-related neurological conditions, anti-HIV antibody positive and HIV-positive conditions, Kaposi's sarcoma, thrombocytopenia purpura and opportunistic infections. In addition, these compounds or formulations can be used prophylactically to prevent or retard the progression of clinical illness in individuals who are anti-HIV antibody or HIV-antigen positive or who have been exposed to HIV.

In certain embodiments a compound of the present invention and its pharmaceutically acceptable derivatives or pharmaceutically acceptable formulations containing these compounds are also useful in the prevention and treatment of HBV infections and other related conditions such as anti-HBV antibody positive and HBV-positive conditions, chronic liver inflammation caused by HBV, cirrhosis, acute hepatitis, fulminant hepatitis, chronic persistent hepatitis, and fatigue. These compounds or formulations can also be used prophylactically to prevent or retard the progression of clinical illness in individuals who are anti-HBV antibody or HBV-antigen positive or who have been exposed to HBV.

In certain embodiments, the condition is associated with an immune response.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

In one non-limiting embodiment compounds of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

In certain embodiments, the condition associated with CDK8 and/or CDK19 kinase activity is a degenerative disorder, e.g., Alzheimer's disease (AD) or Parkinson's Disease.

In another aspect, a method of treating a β-catenin pathway-associated condition is provided comprising administering to a subject in need thereof a compound of the present invention or a pharmaceutically acceptable composition, salt, isotopic analog (such as a deuterated derivative), or prodrug thereof. In another aspect, a method of modulating the J-catenin pathway (e.g., by inhibiting the expression of beta-catenin target genes) in a cell is provided comprising contacting a compound of the present invention or a pharmaceutically acceptable composition, salt, isotopic analog (such as a deuterated derivative), or prodrug thereof. In certain embodiments, the method is an in vitro method. In certain embodiments, the method is an in vivo method.

In another aspect, a method of treating a JAK-STAT pathway-associated condition is provided included administering to a subject in need thereof a compound of the present invention or a pharmaceutically acceptable composition, salt, isotopic analog (such as a deuterated derivative), or prodrug thereof. In another aspect, provided is a method of modulating the STAT1 activity in a cell (e.g., by inhibiting phosphorylation of STAT1 S727 in the JAK-STAT pathway, leading to up- or down-regulation of specific STAT1-associated genes) comprising contacting a compound of the present invention or a pharmaceutically acceptable composition, salt, isotopic analog, or prodrug thereof, with the cell. In certain embodiments, the method is an in vitro method. In certain embodiments, the method is an in vivo method.

It has been reported that nuclear CDKs, such as CDK8, drive SMAD transcriptional activation and turnover in BMP and TGF-beta. See, e.g., Alarcon et al., Cell (2009), 139: 757-769. Thus, in yet another aspect, provided is a method of treating a TGF-beta/BMP pathway-associated condition comprising administering to a subject in need thereof a compound of the present invention or a pharmaceutically acceptable composition, salt, isotopic analog (such as a deuterated derivative) or prodrug thereof. In another aspect, provided is a method of modulating the TGF-beta/BMP pathway (e.g., by inhibiting CDK8/CDK19 phosphorylation SMAD proteins in the TGF-beta/BMP pathway leading to up- or down-regulation of specific SMAD protein-associated genes) in a cell comprising contacting a compound of the present invention or a pharmaceutically acceptable composition, salt, isotopic analog, or prodrug thereof, with the cell. In certain embodiments, the method is an in vitro method. In certain embodiments, the method is an in vivo method.

CDK8 has been linked to regulation of hypoxic response, playing a role in induction of HIF-1-A (HIF-1-alpha) target genes. These genes are involved in angiogenesis, glycolysis, metabolic adaption, and cell survival, processes critical to tumor maintenance and growth. See, e.g., Galbraith, et al., Cell 153:1327-1339. Thus, in one aspect, provided is a method of treating a condition associated with hypoxia comprising administering to a subject in need thereof a compound of the present invention or a pharmaceutically acceptable composition, salt, isotopic analog (such as a deuterated derivative), or prodrug thereof. In another aspect, a method of reducing hypoxia injury is provided comprising administering to a subject in need thereof a compound of the present invention or a pharmaceutically acceptable composition, salt, isotopic analog (such as a deuterated derivative), or prodrug thereof. In yet another aspect, provided is a method of modulating HIF-1-A (HIF-1-alpha) activity (e.g., by inhibiting the expression HIF-1-alpha associated genes) in a cell comprising contacting a compound of the present invention or a pharmaceutically acceptable composition, salt, isotopic analog, or prodrug thereof, with the cell. In certain embodiments, the method is an in vitro method. In certain embodiments, the method is an in vivo method.

In another aspect, a method of increasing BIM expression (e.g., BCLC2L11 expression) is provided to induce apoptosis in a cell comprising contacting a compound of the present invention or a pharmaceutically acceptable composition, salt, isotopic analog, or prodrug thereof with the cell. In certain embodiments, the method is an in vitro method. In certain embodiments, the method is an in vivo method. BCL2L11 expression is tightly regulated in a cell. BCL2L11 encodes for BIM, a proapoptotic protein. BCL2L11 is downregulated in many cancers and BIM is inhibited in many cancers, including chronic myelocytic leukemia (CML) and non-small cell lung cancer (NSCLC) and that suppression of BCL2L11 expression can confer resistance to tyrosine kinase inhibitors. See, e.g., Ng et al., Nat. Med. (2012) 18:521-528.

In yet another aspect, a method of treating a condition associated with angiogenesis is provided, such as, for example, a diabetic condition (e.g., diabetic retinopathy), an inflammatory condition (e.g., rheumatoid arthritis), macular degeneration, obesity, atherosclerosis, or a proliferative disorder, comprising administering to a subject in need thereof a compound of the present invention or a pharmaceutically acceptable composition, salt, isotopic analog, or prodrug thereof.

As used herein, a “diabetic condition” refers to diabetes and pre-diabetes. Diabetes refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger). There are several types of diabetes. Type I diabetes results from the body's failure to produce insulin, and presently requires the person to inject insulin or wear an insulin pump. Type 2 diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e.g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes.

All forms of diabetes increase the risk of long-term complications (referred to herein as the “associated complication” of the diabetic condition). These typically develop after many years, but may be the first symptom in those who have otherwise not received a diagnosis before that time. A major long-term complication relates to damage to blood vessels. Diabetes doubles the risk of cardiovascular disease and macrovascular diseases such as ischemic heart disease (angina, myocardial infarction), stroke, and peripheral vascular disease. Diabetes also causes microvascular complications, e.g., damage to the small blood vessels. Diabetic retinopathy, which affects blood vessel formation in the retina of the eye, can lead to visual symptoms, reduced vision, and potentially blindness. Diabetic nephropathy, the impact of diabetes on the kidneys, can lead to scarring changes in the kidney tissue, loss of small or progressively larger amounts of protein in the urine, and eventually chronic kidney disease requiring dialysis. Diabetic neuropathy is the impact of diabetes on the nervous system, most commonly causing numbness, tingling and pain in the feet and also increasing the risk of skin damage due to altered sensation. Together with vascular disease in the legs, neuropathy contributes to the risk of diabetes-related foot problems, e.g., diabetic foot ulcers that can be difficult to treat and occasionally require amputation.

In certain embodiments, the associated complication is diabetic retinopathy. For example, in certain embodiments, provided is a method of treating diabetic retinopathy comprising administering to a subject in need thereof a compound of the present invention or a pharmaceutically acceptable composition, salt, isotopic analog, or prodrug thereof.

In certain embodiments, the condition associated with angiogenesis is macular degeneration. In certain embodiments, provided is a method of treating macular degeneration comprising administering to a subject in need thereof a compound of the present invention or a pharmaceutically acceptable composition, salt, isotopic analog, or prodrug thereof.

In certain embodiments, the condition associated with angiogenesis is obesity. As used herein, “obesity” and “obese” as used herein, refers to class I obesity, class II obesity, class III obesity and pre-obesity (e.g., being “over-weight”) as defined by the World Health Organization.

In certain embodiments, a method of treating obesity is provided comprising administering to a subject in need thereof a compound of the present invention or a pharmaceutically acceptable composition, salt, isotopic analog, or prodrug thereof.

In certain embodiments, the condition associated with angiogenesis is atherosclerosis. In certain embodiments, provided is a method of treating atherosclerosis comprising administering to a subject in need thereof a compound of the present invention or a pharmaceutically acceptable composition, salt, isotopic analog, or prodrug thereof.

In certain embodiments, the condition associated with angiogenesis is a proliferative disorder. In certain embodiments, provided is a method of treating a proliferative disorder comprising administering to a subject in need thereof a compound of the present invention or a pharmaceutically acceptable composition, salt, isotopic analog, or prodrug thereof.

Exemplary proliferative disorders include, but are not limited to, tumors (e.g., solid tumors), benign neoplasms, pre-malignant neoplasms (carcinoma in situ), and malignant neoplasms (cancers).

Exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In another embodiment, the disorder is myelodysplastic syndrome (MDS).

In certain embodiments, the cancer or tumor is associated with CDK8 and/or CDK19 kinase activity. In certain embodiments, the cancer or tumor is associated with CDK8 kinase activity. In certain embodiments, the cancer or tumor is associated with CDK19 kinase activity. In certain embodiments, the cancer or tumor is associated with aberrant CDK8 kinase activity. In certain embodiments, the cancer or tumor is associated with aberrant CDK19 kinase activity. In certain embodiments, the cancer or tumor is associated with increased CDK8 kinase activity. In certain embodiments, the cancer is associated with increased CDK19 kinase activity.

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

Compounds of the present invention and pharmaceutically acceptable composition, salt, isotopic analog, or prodrug thereof, may be formulated in dosage unit form for ease of administration and uniformity of dosage. It will be understood, however, that the total daily usage of the compositions comprising a compound as described herein will be decided by the attending physician within the scope of sound medical judgment. The specific therapeutically effective dose level for any particular subject or organism will depend upon a variety of factors including the disease, disorder, or condition being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered using any frequency determined to be useful by the health care provider, including three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 0.1 mg to about 10 mg, or about 0.1 mg to about 15 mg, of a compound per unit dosage form. In certain embodiments, an effective amount of an active agent for administration comprises at least about 1 mg, about 5 mg, about 10 mg, about 15 mg, about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1000 mg.

In certain embodiments, the compound may be administered orally or parenterally to an adult human at dosage levels sufficient to deliver from about 0.001 mg/kg to about 100 mg/kg, from about 0.01 mg/kg to about 50 mg/kg, preferably from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 0.01 mg/kg to about 1 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. The compounds or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder (for example, a compound can be administered in combination with an anti-inflammatory agent, anti-cancer agent, etc.), and/or it may achieve different effects (e.g., control of adverse side-effects, e.g., emesis controlled by an anti-emetic).

The compound or composition can be administered concurrently with, prior to, or subsequent to, one or more additional therapeutically active agents. In general, each agent will be administered at a dose and/or on a time schedule determined for that agent. It will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the additional therapeutically active agent is an anti-cancer agent, e.g., radiation therapy and/or one or more chemotherapeutic agents.

### V. Combination Therapy

In one aspect, a treatment regimen is provided comprising the administration of a compound of the present invention or a pharmaceutically acceptable composition, salt, isotopic analog (such as a deuterated derivative), or prodrug thereof in combination or in alternation with at least one additional therapeutic agent. The combinations and/or alternations disclosed herein can be administered for beneficial, additive, or synergistic effect in the treatment of abnormal cellular proliferative disorders.

In one aspect of this embodiment, the second active compound is an immune modulator, including but not limited to a checkpoint inhibitor. Checkpoint inhibitors for use in the methods described herein include, but are not limited to PD-1 inhibitors, PD-L inhibitors, PD-L2 inhibitors, CTLA-4 inhibitors, LAG-3 inhibitors, TIM-3 inhibitors, and V-domain Ig suppressor of T-cell activation (VISTA) inhibitors, or combination thereof.

In one embodiment, the checkpoint inhibitor is a PD-1 inhibitor that blocks the interaction of PD-1 and PD-L1 by binding to the PD-1 receptor, and in turn inhibits immune suppression. In one embodiment, the checkpoint inhibitor is a PD-1 checkpoint inhibitor selected from nivolumab, pembrolizumab, pidilizumab, AMP-224 (AstraZeneca and MedImmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.).

In one embodiment, the checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, avelumab, atezolizumab, durvalumab, KN035, and BMS-936559 (Bristol-Myers Squibb).

In one aspect of this embodiment, the checkpoint inhibitor is a CTLA-4 checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In another embodiment, the checkpoint inhibitor is a LAG-3 checkpoint inhibitor. Examples of LAG-3 checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). In yet another aspect of this embodiment, the checkpoint inhibitor is a TIM-3 checkpoint inhibitor. A specific TIM-3 inhibitor includes, but is not limited to, TSR-022 (Tesaro).

In another embodiment, the compound for use in combination therapy is a LAG-3 targeting ligand. In another embodiment, the compound for use in combination therapy is a TIM-3 targeting ligand. In another embodiment, the compound for use in combination therapy is a aromatase inhibitor. In another embodiment, the compound for use in combination therapy is a progestin receptor targeting ligand. In another embodiment, the compound for use in combination therapy is a CYP3A4 targeting ligand. In another embodiment, the compound for use in combination therapy is a TORC1 or TORC2 targeting ligand.

In specific embodiments, the treatment regimen includes the administration of a compound of the present invention or a pharmaceutically acceptable composition, salt, isotopic analog, or prodrug thereof in combination or alternation with at least one additional kinase inhibitor. In one embodiment, the at least one additional kinase inhibitor is selected from a phosphoinositide 3-kinase (PI3K) inhibitor, a Bruton's tyrosine kinase (BTK) inhibitor, another cyclin-dependent kinase inhibitor, or a spleen tyrosine kinase (Syk) inhibitor, or a combination thereof.

In one embodiment, a compound of the present invention or a pharmaceutically acceptable composition, salt, isotopic analog, or prodrug thereof is combined in a dosage form with the PIk3 inhibitor.

PI3k inhibitors that may be used in the present invention are well known. Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one). AMG-319, GSK-2269557, SAR245409 (N-(4-(N-(3-((3,5-di methoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[l-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methyl sulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), LY3023414, BEZ235 (2-Methyl-2-(4-[3-methyl-2-oxo-8-(quinolin-3-yl)-2,3-dihydro-1H-imidazo[4,5-c]quinolin-1-yl]phenyl)propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)).

BTK inhibitors for use in the present invention are well known. Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference. In one embodiment, a compound of the present invention or a pharmaceutically acceptable composition, salt, isotopic analog, or prodrug thereof is combined in a dosage form with the BTK inhibitor.

In one embodiment the additional cyclin-dependent kinase inhibitor is a CDK7 inhibitor such as THZ1 (N-[3-[[5-chloro-4-(H-indol-3-yl)pyrimidin-2-yl]amino]phenyl]-4-[[(E)-4-(dimethylamino)but-2-enoyl]amino]benzamide). In an alternative embodiment the additional cyclin-dependent kinase inhibitor is a CDK9 inhibitor such as flavopiridol (alvocidib).

Therefore in one embodiment, a method of treating a tumor or cancer is provided, comprising administration of an effective amount of Compound B or a pharmaceutically acceptable salt thereof in combination or alternation with an effective amount of a Syk inhibitor to a host in need thereof. Alternatively, a method of treating a tumor or cancer is provided, comprising administration of an effective amount of Compound C in combination or alternation with an effective amount of a Syk inhibitor to a host in need thereof. Alternatively, a method of treating a tumor or cancer is provided, comprising administration of an effective amount of Compound D or a pharmaceutically acceptable salt thereof in combination or alternation with an effective amount of a Syk inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a Syk inhibitor to a host in need thereof. Alternatively, a method of treating a tumor or cancer is provided, comprising administration of an effective amount of an analog of Compound B as provided herein in combination or alternation with an effective amount of a Syk inhibitor to a host in need thereof. Alternatively, a method of treating a tumor or cancer is provided, comprising administration of an effective amount of an analog of Compound C or a pharmaceutically acceptable salt thereof as provided herein in combination or alternation with an effective amount of a Syk inhibitor to a host in need thereof. Alternatively, a method of treating a tumor or cancer is provided, comprising administration of an effective amount of an analog of Compound D or a pharmaceutically acceptable salt thereof as provided herein in combination or alternation with an effective amount of a Syk inhibitor to a host in need thereof.

In one embodiment, a method of treating a tumor or cancer is provided, comprising administration of an effective amount of Compound B or a pharmaceutically acceptable salt thereof in combination or alternation with imatinib (Gleevec) to a host in need thereof. Alternatively, a method of treating a tumor or cancer is provided, comprising administration of an effective amount of Compound C or a pharmaceutically acceptable salt thereof in combination or alternation with imatinib (Gleevec) to a host in need thereof. Alternatively, a method of treating a tumor or cancer is provided, comprising administration of an effective amount of Compound D or a pharmaceutically acceptable salt thereof in combination or alternation with imatinib (Gleevec) to a host in need thereof. In another embodiment, a method of treating a tumor or cancer is provided, comprising administration of an effective amount of an analog of Compound A or a pharmaceutically acceptable salt thereof as provided herein in combination or alternation with imatinib (Gleevec) to a host in need thereof. Alternatively, a method of treating a tumor or cancer is provided, comprising administration of an effective amount of an analog of Compound B or a pharmaceutically acceptable salt thereof as provided herein in combination or alternation with imatinib (Gleevec) to a host in need thereof. Alternatively, a method of treating a tumor or cancer is provided, comprising administration of an effective amount of an analog of Compound C or a pharmaceutically acceptable salt thereof as provided herein in combination or alternation with imatinib (Gleevec) to a host in need thereof. Alternatively, a method of treating a tumor or cancer is provided, comprising administration of an effective amount of an analog of Compound D or a pharmaceutically acceptable salt thereof as provided herein in combination or alternation with imatinib (Gleevec) to a host in need thereof.

Syk inhibitors for use in the present invention are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevec; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein). In one embodiment a compound of the present invention or a pharmaceutically acceptable composition, salt, isotopic analog, or prodrug thereof is combined in a dosage form with the Syk inhibitor.

In specific embodiments, the method of treatment provided includes the administration of a compound of the present invention or a pharmaceutically acceptable composition, salt, isotopic analog, or prodrug thereof in combination or alternation with at least one additional chemotherapeutic agent.

In one embodiment, the at least one additional chemotherapeutic agent combined or alternated with a compound of the present invention is a protein cell death-1 (PD-1) inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech). In one embodiment, a compound of the present invention or a pharmaceutically acceptable composition, salt, isotopic analog, or prodrug thereof is combined in a dosage form with the PD-1 inhibitor. In one embodiment the PD-1 inhibitor is pembrolizumab.

In one embodiment, a method of treating a tumor or cancer is provided, comprising administration of an effective amount of Compound B or a pharmaceutically acceptable salt thereof in combination or alternation with an effective amount of a PD-1 inhibitor to a host in need thereof. Alternatively, a method of treating a tumor or cancer is provided, comprising administration of an effective amount of Compound C or a pharmaceutically acceptable salt thereof in combination or alternation with an effective amount of a PD-1 inhibitor to a host in need thereof. Alternatively, a method of treating a tumor or cancer is provided, comprising administration of an effective amount of Compound D or a pharmaceutically acceptable salt thereof in combination or alternation with an effective amount of a PD-1 inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a PD-1 inhibitor to a host in need thereof. Alternatively, a method of treating a tumor or cancer is provided, comprising administration of an effective amount of an analog of Compound B or a pharmaceutically acceptable salt thereof as provided herein in combination or alternation with an effective amount of a PD-1 inhibitor to a host in need thereof. Alternatively, a method of treating a tumor or cancer is provided, comprising administration of an effective amount of an analog of Compound C or a pharmaceutically acceptable salt thereof as provided herein in combination or alternation with an effective amount of a PD-1 inhibitor to a host in need thereof. Alternatively, a method of treating a tumor or cancer is provided, comprising administration of an effective amount of an analog of Compound D or a pharmaceutically acceptable salt thereof as provided herein in combination or alternation with an effective amount of a PD-1 inhibitor to a host in need thereof.

In one embodiment, a method of treating a tumor or cancer is provided, comprising administration of an effective amount of Compound B or a pharmaceutically acceptable salt thereof in combination or alternation with pembrolizumab (Keytruda). Alternatively, a method of treating a tumor or cancer is provided, comprising administration of an effective amount of Compound C or a pharmaceutically acceptable salt thereof in combination or alternation with pembrolizumab (Keytruda). Alternatively, a method of treating a tumor or cancer is provided, comprising administration of an effective amount of Compound D or a pharmaceutically acceptable salt thereof in combination or alternation with pembrolizumab (Keytruda). In another embodiment, a method of treating a tumor or cancer is provided, comprising administration of an effective amount of an analog of Compound A or a pharmaceutically acceptable salt thereof as provided herein in combination or alternation with pembrolizumab (Keytruda). Alternatively, a method of treating a tumor or cancer is provided, comprising administration of an effective amount of an analog of Compound B or a pharmaceutically acceptable salt thereof as provided herein in combination or alternation with pembrolizumab (Keytruda). Alternatively, a method of treating a tumor or cancer is provided, comprising administration of an effective amount of an analog of Compound C or a pharmaceutically acceptable salt thereof as provided herein in combination or alternation with pembrolizumab (Keytruda). Alternatively, a method of treating a tumor or cancer is provided, comprising administration of an effective amount of an analog of Compound D or a pharmaceutically acceptable salt thereof as provided herein in combination or alternation with pembrolizumab (Keytruda).

In one embodiment, the at least one additional chemotherapeutic agent combined or alternated with a compound of the present invention is a CTLA-4 inhibitor. CTLA-4 inhibitors are known in the art, and include, for example, ipilimumab (Yervoy) marketed by Bristol-Myers Squibb and tremelimumab marketed by Pfizer.

In one embodiment, the at least one additional chemotherapeutic agent combined or alternated with the compound of the present invention is a BET inhibitor. BET inhibitors are known in the art, and include, for example, JQ1, I-BET 151 (a.k.a. GSK1210151A), I-BET 762 (a.k.a. GSK525762), OTX-015 (a.k.a. MK-8268, IUPAC 6H-Thieno[3,2-f][1,2,4]triazolo[4,3-a][1,4]diazepine-6-acetamide, 4-(4-chlorophenyl)-N-(4-hydroxyphenyl)-2,3,9-trimethyl-), TEN-010, CPI-203, CPI-0610, RVX-208, and LY294002. In one embodiment the BET inhibitor used in combination or alternation with a compound of the present invention for treatment of a tumor or cancer is JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate). In an alternative embodiment the BET inhibitor used in combination or alternation with a compound of the present invention for treatment of a tumor or cancer is I-BET 151 (2H-Imidazo[4,5-c]quinolin-2-one, 7-(3,5-dimethyl-4-isoxazolyl)-1,3-dihydro-8-methoxy-1-[(1R)-1-(2-pyridinyl)ethyl]-).

In one embodiment, the additional active agent is the small molecule BET inhibitor, MK-8628 (CAS 202590-98-5) (6H-thieno(3,2-f)-(1,2,4)triazolo(4,3-a)-(1,4)diazepine-6-acetamide, 4-(4-chlorophenyl)-N-(4-hydroxyphenyl)2,3,9-trimethyl, (6S).

In one embodiment, a method of treating a tumor or cancer is provided, comprising administration of an effective amount of Compound B or a pharmaceutically acceptable salt thereof in combination or alternation with an effective amount of a BET inhibitor to a host in need thereof. Alternatively, a method of treating a tumor or cancer is provided, comprising administration of an effective amount of Compound C or a pharmaceutically acceptable salt thereof in combination or alternation with an effective amount of a BET inhibitor to a host in need thereof. Alternatively, a method of treating a tumor or cancer is provided, comprising administration of an effective amount of Compound D or a pharmaceutically acceptable salt thereof in combination or alternation with an effective amount of a BET inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a BET inhibitor to a host in need thereof. Alternatively, a method of treating a tumor or cancer is provided, comprising administration of an effective amount of an analog of Compound B or a pharmaceutically acceptable salt thereof as provided herein in combination or alternation with an effective amount of a BET inhibitor to a host in need thereof. Alternatively, a method of treating a tumor or cancer is provided, comprising administration of an effective amount of an analog of Compound C or a pharmaceutically acceptable salt thereof as provided herein in combination or alternation with an effective amount of a BET inhibitor to a host in need thereof. Alternatively, a method of treating a tumor or cancer is provided, comprising administration of an effective amount of an analog of Compound D or a pharmaceutically acceptable salt thereof as provided herein in combination or alternation with an effective amount of a BET inhibitor to a host in need thereof.

In one embodiment, a method of treating a tumor or cancer is provided, comprising administration of an effective amount of Compound B or a pharmaceutically acceptable salt thereof in combination or alternation with JQ1. Alternatively, a method of treating a tumor or cancer is provided, comprising administration of an effective amount of Compound C or a pharmaceutically acceptable salt thereof in combination or alternation with JQ1. Alternatively, a method of treating a tumor or cancer is provided, comprising administration of an effective amount of Compound D or a pharmaceutically acceptable salt thereof in combination or alternation with JQ1. In another embodiment, a method of treating a tumor or cancer is provided, comprising administration of an effective amount of an analog of Compound A or a pharmaceutically acceptable salt thereof as provided herein in combination or alternation with JQ1. Alternatively, a method of treating a tumor or cancer is provided, comprising administration of an effective amount of an analog of Compound B or a pharmaceutically acceptable salt thereof as provided herein in combination or alternation with JQ1. Alternatively, a method of treating a tumor or cancer is provided, comprising administration of an effective amount of an analog of Compound C or a pharmaceutically acceptable salt thereof as provided herein in combination or alternation with JQ1. Alternatively, a method of treating a tumor or cancer is provided, comprising administration of an effective amount of an analog of Compound D or a pharmaceutically acceptable salt thereof as provided herein in combination or alternation with JQ1.

In one embodiment, a method of treating a tumor or cancer is provided, comprising administration of an effective amount of Compound B or a pharmaceutically acceptable salt thereof in combination or alternation with I-BET 151. Alternatively, a method of treating a tumor or cancer is provided, comprising administration of an effective amount of Compound C or a pharmaceutically acceptable salt thereof in combination or alternation with I-BET 151. Alternatively, a method of treating a tumor or cancer is provided, comprising administration of an effective amount of Compound D or a pharmaceutically acceptable salt thereof in combination or alternation with I-BET 151. In another embodiment, a method of treating a tumor or cancer is provided, comprising administration of an effective amount of an analog of Compound A or a pharmaceutically acceptable salt thereof as provided herein in combination or alternation with I-BET 151. Alternatively, a method of treating a tumor or cancer is provided, comprising administration of an effective amount of an analog of Compound B or a pharmaceutically acceptable salt thereof as provided herein in combination or alternation with I-BET 151. Alternatively, a method of treating a tumor or cancer is provided, comprising administration of an effective amount of an analog of Compound C or a pharmaceutically acceptable salt thereof as provided herein in combination or alternation with I-BET 151. Alternatively, a method of treating a tumor or cancer is provided, comprising administration of an effective amount of an analog of Compound D or a pharmaceutically acceptable salt thereof as provided herein in combination or alternation with I-BET 151.

In one embodiment, the at least one additional chemotherapeutic agent combined or alternated with the compound of the present invention is a MEK inhibitor. MEK inhibitors for use in the present invention are well known, and include, for example, tametinib/GSK1 120212 (N-(3-{{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-((3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK 162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1, and 5-dimethyl-6-oxo-1,6-dihydropyridine-3-carboxamide). In one embodiment, a compound of the present invention or a pharmaceutically acceptable composition, salt, isotopic analog, or prodrug thereof is combined in a dosage form with the MEK inhibitor.

In one embodiment, the at least one additional chemotherapeutic agent combined or alternated with the compound of the present invention is a Raf inhibitor. Raf inhibitors for use in the present invention are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide). In one embodiment, a compound of the present invention or a pharmaceutically acceptable composition, salt, isotopic analog, or prodrug thereof is combined in a dosage form with the Raf inhibitor.

In one embodiment, the at least one additional chemotherapeutic agent combined or alternated with the compound of the present invention is a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl] amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX 15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen). In one embodiment, a compound of the present invention or a pharmaceutically acceptable composition, salt, isotopic analog, or prodrug thereof is combined in a dosage form with the at least one BCL-2 inhibitor. In one embodiment the at least one BCL-2 inhibitor is ABT-199 (Venetoclax).

In one embodiment, a method of treating a tumor or cancer is provided, comprising administration of an effective amount of Compound B or a pharmaceutically acceptable salt thereof in combination or alternation with an effective amount of a BCL-2 inhibitor to a host in need thereof. Alternatively, a method of treating a tumor or cancer is provided, comprising administration of an effective amount of Compound C or a pharmaceutically acceptable salt thereof in combination or alternation with an effective amount of a BCL-2 inhibitor to a host in need thereof. Alternatively, a method of treating a tumor or cancer is provided, comprising administration of an effective amount of Compound D or a pharmaceutically acceptable salt thereof in combination or alternation with an effective amount of a BCL-2 inhibitor to a host in need thereof. In another embodiment, a method of treating a tumor or cancer is provided, comprising administration of an effective amount of an analog of Compound A or a pharmaceutically acceptable salt thereof as provided herein in combination or alternation with an effective amount of a BCL-2 inhibitor to a host in need thereof. Alternatively, a method of treating a tumor or cancer is provided, comprising administration of an effective amount of an analog of Compound B or a pharmaceutically acceptable salt thereof as provided herein in combination or alternation with an effective amount of a BCL-2 inhibitor to a host in need thereof. Alternatively, a method of treating a tumor or cancer is provided, comprising administration of an effective amount of an analog of Compound C or a pharmaceutically acceptable salt thereof as provided herein in combination or alternation with an effective amount of a BCL-2 inhibitor to a host in need thereof. Alternatively, a method of treating a tumor or cancer is provided, comprising administration of an effective amount of an analog of Compound D or a pharmaceutically acceptable salt thereof as provided herein in combination or alternation with an effective amount of a BCL-2 inhibitor to a host in need thereof.

In one embodiment, a method of treating a tumor or cancer is provided, comprising administration of an effective amount of Compound B or a pharmaceutically acceptable salt thereof in combination or alternation with ABT-199 to a host in need thereof. Alternatively, a method of treating a tumor or cancer is provided, comprising administration of an effective amount of Compound C or a pharmaceutically acceptable salt thereof in combination or alternation with ABT-199 to a host in need thereof. Alternatively, a method of treating a tumor or cancer is provided, comprising administration of an effective amount of Compound D or a pharmaceutically acceptable salt thereof in combination or alternation with ABT-199 to a host in need thereof. In another embodiment, a method of treating a tumor or cancer is provided, comprising administration of an effective amount of an analog of Compound A or a pharmaceutically acceptable salt thereof as provided herein in combination or alternation with ABT-199 to a host in need thereof. Alternatively, a method of treating a tumor or cancer is provided, comprising administration of an effective amount of an analog of Compound B or a pharmaceutically acceptable salt thereof as provided herein in combination or alternation with ABT-199 to a host in need thereof. Alternatively, a method of treating a tumor or cancer is provided, comprising administration of an effective amount of an analog of Compound C or a pharmaceutically acceptable salt thereof as provided herein in combination or alternation with ABT-199 to a host in need thereof. Alternatively, a method of treating a tumor or cancer is provided, comprising administration of an effective amount of an analog of Compound D or a pharmaceutically acceptable salt thereof as provided herein in combination or alternation with ABT-199 to a host in need thereof.

In one embodiment, the treatment regimen includes the administration of a compound of the present invention or a pharmaceutically acceptable composition, salt, isotopic analog, or prodrug thereof in combination or alternation with at least one additional chemotherapeutic agent selected from, but are not limited to, Imatinib mesylate (Gleevac), Dasatinib (Sprycel), Nilotinib (Tasigna), Bosutinib (Bosulif), Trastuzumab (Herceptin), Pertuzumab (Perjeta™), Lapatinib (Tykerb), Gefitinib (Iressa), Erlotinib (Tarceva), Cetuximab (Erbitux), Panitumumab (Vectibix), Vandetanib (Caprelsa), Vemurafenib (Zelboraf), Vorinostat (Zolinza), Romidepsin (Istodax), Bexarotene (Tagretin), Alitretinoin (Panretin), Tretinoin (Vesanoid), Carfilizomib (Kyprolis™), Pralatrexate (Folotyn), Bevacizumab (Avastin), Ziv-aflibercept (Zaltrap), Sorafenib (Nexavar), Sunitinib (Sutent), Pazopanib (Votrient), Regorafenib (Stivarga), and Cabozantinib (Cometriq™).

In some embodiments, the pharmaceutical combination or composition described herein can be administered to the subject in combination or further combination with other chemotherapeutic agents for the treatment of a tumor or cancer. If convenient, the pharmaceutical combination or composition described herein can be administered at the same time as another chemotherapeutic agent, in order to simplify the treatment regimen. In some embodiments, the pharmaceutical combination or composition and the other chemotherapeutic can be provided in a single formulation. In one embodiment, the use of the pharmaceutical combination or composition described herein is combined in a therapeutic regime with other agents. Such agents may include, but are not limited to, tamoxifen, midazolam, letrozole, bortezomib, anastrozole, goserelin, an mTOR inhibitor, a PI3 kinase inhibitor as described above, a dual mTOR-PI3K inhibitor, a MEK inhibitor as described above, a RAS inhibitor, ALK inhibitor, an HSP inhibitor (for example, HSP70 and HSP 90 inhibitor, or a combination thereof), a BCL-2 inhibitor as described above, apopototic inducing compounds, an AKT inhibitor, including but not limited to, MK-2206 (1,2,4-Triazolo[3,4-f][1,6]naphthyridin-3(2H)-one, 8-[4-(1-aminocyclobutyl)phenyl]-9-phenyl-), GSK690693, Perifosine, (KRX-0401), GDC-0068, Triciribine, AZD5363, Honokiol, PF-04691502, and Miltefosine, a PD-1 inhibitor as described above including but not limited to, Nivolumab, CT-011, MK-3475, BMS936558, and AMP-514 or a FLT-3 inhibitor, including but not limited to, P406, Dovitinib, Quizartinib (AC220), Amuvatinib (MP-470), Tandutinib (MLN518), ENMD-2076, and KW-2449, or a combination thereof. Examples of mTOR inhibitors include but are not limited to rapamycin and its analogs, everolimus (Afinitor), temsirolimus, ridaforolimus, sirolimus, and deforolimus. Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol. In a particular embodiment, a compound described herein is administered in combination with letrozole and/or tamoxifen. Other chemotherapeutic agents that can be used in combination with the compounds described herein include, but are not limited to, chemotherapeutic agents that do not require cell cycle activity for their anti-neoplastic effect.

In one embodiment, the treatment regimen includes the administration of a compound of the present invention or a pharmaceutically acceptable composition, salt, isotopic analog, or prodrug thereof in combination or alternation with at least one additional therapy. The second therapy can be an immunotherapy. As discussed in more detail below, the combination agent can be conjugated to an antibody, radioactive agent, or other targeting agent that directs the active compound as described herein to the diseased or abnormally proliferating cell. In another embodiment, the pharmaceutical combination or composition is used in combination with another pharmaceutical or a biologic agent (for example an antibody) to increase the efficacy of treatment with a combined or a synergistic approach. In an embodiment, the pharmaceutical combination or composition can be used with T-cell vaccination, which typically involves immunization with inactivated autoreactive T cells to eliminate a cancer cell population as described herein. In another embodiment, the pharmaceutical combination or composition is used in combination with a bispecific T-cell Engager (BiTE), which is an antibody designed to simultaneously bind to specific antigens on endogenous T cells and cancer cells as described herein, linking the two types of cells.

In one embodiment, the additional therapy is a monoclonal antibody (MAb). Some MAbs stimulate an immune response that destroys cancer cells. Similar to the antibodies produced naturally by B cells, these MAbs “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

Another group of cancer therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a cancer cell, and the cell-killing substance is taken up by the cell. FDA-approved conjugated MAbs that work this way include ado-trastuzumab emtansine, which targets the HER-2 molecule to deliver the drug DM1, which inhibits cell proliferation, to HER-2 expressing metastatic breast cancer cells.

Immunotherapies with T cells engineered to recognize cancer cells via bispecific antibodies (bsAbs) or chimeric antigen receptors (CARs) are approaches with potential to ablate both dividing and non/slow-dividing subpopulations of cancer cells.

Bispecific antibodies, by simultaneously recognizing target antigen and an activating receptor on the surface of an immune effector cell, offer an opportunity to redirect immune effector cells to kill cancer cells. Another approach is the generation of chimeric antigen receptors by fusing extracellular antibodies to intracellular signaling domains. Chimeric antigen receptor-engineered T cells are able to specifically kill tumor cells in a MHC-independent way.

In certain aspects, the additional therapy is another therapeutic agent, for example, an anti-inflammatory agent, a chemotherapeutic agent, a radiotherapeutic agent, or an immunosuppressive agent.

Suitable chemotherapeutic agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin) or liposomal vincristine (Marqibo), Daunorubicin (daunomycin or Cerubidine) or doxorubicin (Adriamycin), Cytarabine (cytosine arabinoside, ara-C, or Cytosar), L-asparaginase (Elspar) or PEG-L-asparaginase (pegaspargase or Oncaspar), Etoposide (VP-16), Teniposide (Vumon), 6-mercaptopurine (6-MP or Purinethol), Methotrexate, Cyclophosphamide (Cytoxan), Prednisone, Dexamethasone (Decadron), imatinib (Gleevec marketed by Novartis), dasatinib (Sprycel), nilotinib (Tasigna), bosutinib (Bosulif), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Suitable immunosuppressive agents include, but are not limited to: calcineurin inhibitors, e.g. a cyclosporin or an ascomycin, e.g. Cyclosporin A (NEORAL), FK506 (tacrolimus), pimecrolimus, a mTOR inhibitor, e.g. rapamycin or a derivative thereof, e.g. Sirolimus (RAPAMUNE), Everolimus (Certican), temsirolimus, zotarolimus, biolimus-7, biolimus-9, a rapalog, e.g.ridaforolimus, azathioprine, campath 1H, a SIP receptor modulator, e.g. fingolimod or an analog thereof, an anti 1L-8 antibody, mycophenolic acid or a salt thereof, e.g. sodium salt, or a prodrug thereof, e.g. Mycophenolate Mofetil (CELLCEPT), OKT3 (ORTHOCLONE OKT3), Prednisone, ATGAM, THYMOGLOBULIN, Brequinar Sodium, OKT4, T10B9.A-3A, 33B3.1, 15-deoxyspergualin, tresperimus, Leflunomide ARAVA, CTLAI-Ig, anti-CD25, anti-IL2R, Basiliximab (SIMULECT), Daclizumab (ZENAPAX), mizorbine, methotrexate, dexamethasone, ISAtx-247, SDZ ASM 981 (pimecrolimus, Elidel), CTLA41g (Abatacept), belatacept, LFA31g, etanercept (sold as Enbrel by Immunex), adalimumab (Humira), infliximab (Remicade), an anti-LFA-1 antibody, natalizumab (Antegren), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In certain embodiments, a pharmaceutical combination or composition described herein is administered to the subject prior to treatment with another chemotherapeutic agent, during treatment with another chemotherapeutic agent, after administration of another chemotherapeutic agent, or a combination thereof.

In some embodiments, the selective pharmaceutical combination or composition can be administered to the subject such that the other chemotherapeutic agent can be administered either at higher doses (increased chemotherapeutic dose intensity) or more frequently (increased chemotherapeutic dose density). Dose-dense chemotherapy is a chemotherapy treatment plan in which drugs are given with less time between treatments than in a standard chemotherapy treatment plan. Chemotherapy dose intensity represents unit dose of chemotherapy administered per unit time. Dose intensity can be increased or decreased through altering dose administered, time interval of administration, or both.

In one embodiment of the invention, the pharmaceutical combination or composition described herein can be administered in a concerted regimen with another agent such as a non-DNA-damaging, targeted anti-neoplastic agent or a hematopoietic growth factor agent. It has recently been reported that the untimely administration of hematopoietic growth factors can have serious side effects. For example, the use of the EPO family of growth factors has been associated with arterial hypertension, cerebral convulsions, hypertensive encephalopathy, thromboembolism, iron deficiency, influenza like syndromes and venous thrombosis. The G-CSF family of growth factors has been associated with spleen enlargement and rupture, respiratory distress syndrome, allergic reactions and sickle cell complications. By combining the administration of the pharmaceutical combination or composition as described herein with the timely administration of hematopoietic growth factors, for example, at the time point wherein the affected cells are no longer under growth arrest, it is possible for the health care practitioner to decrease the amount of the growth factor to minimize the unwanted adverse effects while achieving the desired therapeutic benefit. As such, in one embodiment, the use of the pharmaceutical combination, composition, or methods described herein is combined with the use of hematopoietic growth factors including, but not limited to, granulocyte colony stimulating factor (G-CSF, for example, sold as Neupogen (filgrastin), Neulasta (peg-filgrastin), or lenograstin), granulocyte-macrophage colony stimulating factor (GM-CSF, for example sold as molgramostim and sargramostim (Leukine)), M-CSF (macrophage colony stimulating factor), thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-α as Darbopoetin, Epocept, Nanokine, Epofit, Epogin, Eprex and Procrit; epoetin-3 sold as for example NeoRecormon, Recormon and Micera), epoetin-delta (sold as for example Dynepo), epoetin-omega (sold as for example Epomax), epoetin zeta (sold as for example Silapo and Reacrit) as well as for example Epocept, EPOTrust, Erypro Safe, Repoeitin, Vintor, Epofit, Erykine, Wepox, Espogen, Relipoeitin, Shanpoietin, Zyrop and EPIAO). In one embodiment, the pharmaceutical combination or composition is administered prior to administration of the hematopoietic growth factor. In one embodiment, the hematopoietic growth factor administration is timed so that the pharmaceutical combination or composition's effect on HSPCs has dissipated. In one embodiment, the growth factor is administered at least 20 hours after the administration of a pharmaceutical combination or composition described herein.

If desired, multiple doses of a pharmaceutical combination or composition described herein can be administered to the subject. Alternatively, the subject can be given a single dose of a pharmaceutical combination or composition described herein.

In one embodiment, the activity of an active compound for a purpose described herein can be augmented through conjugation to an agent that targets the diseased or abnormally proliferating cell or otherwise enhances activity, delivery, pharmacokinetics or other beneficial property.

A selected compound described herein can be administered in conjugation or combination with a Fv fragment. Fv fragments are the smallest fragment made from enzymatic cleavage of IgG and IgM class antibodies. Fv fragments have the antigen-binding site made of the VH and VC regions, but they lack the CH1 and CL regions. The VH and VL chains are held together in Fv fragments by non-covalent interactions.

In one embodiment, a selected compound as described herein can be administered in combination with an antibody fragment selected from the group consisting of an ScFv, domain antibody, diabody, triabody, tetrabody, Bis-scFv, minibody, Fab2, or Fab3 antibody fragment. In one embodiment, the antibody fragment is a ScFv. Genetic engineering methods allow the production of single chain variable fragments (ScFv), which are Fv type fragments that include the VH and VL domains linked with a flexible peptide When the linker is at least 12 residues long, the ScFv fragments are primarily monomeric. Manipulation of the orientation of the V-domains and the linker length creates different forms of Fv molecules linkers that are 3-11 residues long yield scFv molecules that are unable to fold into a functional Fv domain. These molecules can associate with a second scFv molecule, to create a bivalent diabody. In one embodiment, the antibody fragment administered in combination with a selected compound described herein is a bivalent diabody. If the linker length is less than three residues, scFv molecules associate into triabodies or tetrabodies. In one embodiment, the antibody fragment is a triabody. In one embodiment, the antibody fragment is a tetrabody. Multivalent scFvs possess greater functional binding affinity to their target antigens than their monovalent counterparts by having binding to two more target antigens, which reduces the off-rate of the antibody fragment. In one embodiment, the antibody fragment is a minibody. Minibodies are scFv-CH3 fusion proteins that assemble into bivalent dimers. In one embodiment, the antibody fragment is a Bis-scFv fragment. Bis-scFv fragments are bispecific. Miniaturized ScFv fragments can be generated that have two different variable domains, allowing these Bis-scFv molecules to concurrently bind to two different epitopes.

In one embodiment, a selected compound described herein is administered in conjugation or combination with a bispecific dimer (Fab2) or trispecific dimer (Fab3). Genetic methods are also used to create bispecific Fab dimers (Fab2) and trispecific Fab trimers (Fab3). These antibody fragments are able to bind 2 (Fab2) or 3 (Fab3) different antigens at once.

In one embodiment, a selected compound described herein is administered in conjugation or combination with an rIgG antibody fragment. rIgG antibody fragments refers to reduced IgG (75,000 daltons) or half-IgG. It is the product of selectively reducing just the hinge-region disulfide bonds. Although several disulfide bonds occur in IgG, those in the hinge-region are most accessible and easiest to reduce, especially with mild reducing agents like 2-mercaptoethylamine (2-MEA). Half-IgG are frequently prepared for the purpose of targeting the exposing hinge-region sulfhydryl groups that can be targeted for conjugation, either antibody immobilization or enzyme labeling.

In other embodiments, a selected active compound described herein can be linked to a radioisotope to increase efficacy, using methods well known in the art. Any radioisotope that is useful against cancer cells can be incorporated into the conjugate, for example, but not limited to, ¹³¹I, ¹²³I, ¹⁹²Ir, ³²P, ⁹⁰Sr, ¹⁹⁸Au, ²²⁶Ra, ⁹⁰Y, ²⁴¹Am, ²⁵²Cf, ⁶⁰Co, or ¹³⁷Cs.

Of note, the linker chemistry can be important to efficacy and tolerability of the drug conjugates. The thio-ether linked T-DM1 increases the serum stability relative to a disulfide linker version and appears to undergo endosomal degradation, resulting in intra-cellular release of the cytotoxic agent, thereby improving efficacy and tolerability, See, Barginear, M. F. and Budman, D. R., Trastuzumab-DM1: A review of the novel immune-conjugate for HER2-overexpressing breast cancer, The Open Breast Cancer Journal, 1: 25-30, (2009). Examples of early and recent antibody-drug conjugates, discussing drugs, linker chemistries and classes of targets for product development that may be used in the present invention can be found in the reviews by Casi, G. and Neri, D., Antibody-drug conjugates: basic concepts, examples and future perspectives, J. Control Release 161(2):422-428, 2012, Chari, R. V., Targeted cancer therapy: conferring specificity to cytotoxic drugs, Acc. Chem. Rev., 41(1):98-107, 2008, Sapra, P. and Shor, B., Monoclonal antibody-based therapies in cancer: advances and challenges, Pharmacol. Ther., 138(3):452-69, 2013, Schliemann, C. and Neri, D., Antibody-based targeting of the tumor vasculature, Biochim. Biophys. Acta., 1776(2): 175-92, 2007, Sun, Y., Yu, F., and Sun, B. W., Antibody-drug conjugates as targeted cancer therapeutics, Yao Xue Xue Bao, 44(9):943-52, 2009, Teicher, B. A., and Chari, R. V., Antibody conjugate therapeutics: challenges and potential, Clin. Cancer Res., 17(20):6389-97, 2011, Firer, M. A., and Gellerman, G. J., Targeted drug delivery for cancer therapy: the other side of antibodies, J. Hematol. Oncol., 5:70, 2012, Vlachakis, D. and Kossida, S., Antibody Drug Conjugate bioinformatics: drug delivery through the letterbox, Comput. Math. Methods Med., 2013; 2013:282398, Epub 2013 Jun. 19, Lambert, J. M., Drug-conjugated antibodies for the treatment of cancer, Br. J. Clin. Pharmacol., 76(2):248-62, 2013, Concalves, A., Tredan, O., Villanueva, C. and Dumontet, C., Antibody-drug conjugates in oncology: from the concept to trastuzumab emtansine (T-DM1), Bull. Cancer, 99(12):1183-1191, 2012, Newland, A. M., Brentuximab vedotin: a CD-30-directed antibody-cytotoxic drug conjugate, Pharmacotherapy, 33(1):93-104, 2013, Lopus, M., Antibody-DM1 conjugates as cancer therapeutics, Cancer Lett., 307(2):113-118, 2011, Chu, Y. W. and Poison, A., Antibody-drug conjugates for the treatment of B-cell non-Hodgkin's lymphoma and leukemia, Future Oncol., 9(3):355-368, 2013, Bertholjotti, I., Antibody-drug conjugate a new age for personalized cancer treatment, Chimia, 65(9): 746-748, 2011, Vincent, K. J., and Zurini, M., Current strategies in antibody engineering: Fc engineering and pH-dependent antigen binding, bispecific antibodies and antibody drug conjugates, Biotechnol. J., 7(12): 1444-1450, 2012, Haeuw, J. F., Caussanel, V., and Beck, A., Immunoconjugates, drug-armed antibodies to fight against cancer, Med. Sci., 25(12):1046-1052, 2009 and Govindan, S. V., and Goldenberg, D. M., Designing immunoconjugates for cancer therapy, Expert Opin. Biol. Ther., 12(7):873-890, 2012.

In one embodiment the pharmaceutical composition or combination as described herein can be used to treat any disorder described herein.

In one aspect a compound of the present invention is dosed in a combination or composition with an effective amount of a nucleoside or nucleoside analog. Non-limiting examples of nucleosides include: azacitidine, decitabine, didanosine, vidarabine, BCX4430, cytarabine, emtricitabine, lamivudine, zalcitabine, abacavir, aciclovir, entecavir, stavudine, telbivudine, zidovudine, idoxuridine, trifluridine, apricitabine, elvucitabine, amdoxovir, and racivir. In one embodiment the compound of present invention is used in a combination or composition with an effective amount of a nucleoside or nucleoside analog to treat a viral infection. In an alternative embodiment the compound of present invention is used in a combination or composition with an effective amount of a nucleoside or nucleoside analog to treat a tumor or cancer. In one embodiment the nucleoside analog is azacitidine and the disorder is tumor or cancer.

In one embodiment, provided is a method of treating tumor or cancer in a subject comprising administration of Compound B or a pharmaceutically acceptable salt thereof in combination or alternation with an effective amount of a nucleoside analog to a host in need thereof. Alternatively, provided is a method of treating tumor or cancer in a subject comprising administration of Compound C or a pharmaceutically acceptable salt thereof in combination or alternation with an effective amount of a nucleoside analog to a host in need thereof. Alternatively, provided is a method of treating tumor or cancer in a subject comprising administration of Compound D or a pharmaceutically acceptable salt thereof in combination or alternation with an effective amount of a nucleoside analog to a host in need thereof. In another embodiment, provided is a method of treating tumor or cancer in a subject comprising administration of an analog of Compound A or a pharmaceutically acceptable salt thereof as provided herein in combination or alternation with an effective amount of a nucleoside analog to a host in need thereof. Alternatively, provided is a method of treating tumor or cancer in a subject comprising administration of an analog of Compound B or a pharmaceutically acceptable salt thereof as provided herein in combination or alternation with an effective amount of a nucleoside analog to a host in need thereof. Alternatively, provided is a method of treating tumor or cancer in a subject comprising administration of an analog of Compound C or a pharmaceutically acceptable salt thereof as provided herein in combination or alternation with an effective amount of a nucleoside analog to a host in need thereof. Alternatively, provided is a method of treating tumor or cancer in a subject comprising administration of an analog of Compound D or a pharmaceutically acceptable salt thereof as provided herein in combination or alternation with an effective amount of a nucleoside analog to a host in need thereof.

In one embodiment, provided is a method of treating tumor or cancer in a subject comprising administration of Compound B or a pharmaceutically acceptable salt thereof in combination or alternation with azacitidine to a host in need thereof. Alternatively, provided is a method of treating tumor or cancer in a subject comprising administration of Compound C or a pharmaceutically acceptable salt thereof in combination or alternation with azacitidine to a host in need thereof. Alternatively, provided is a method of treating tumor or cancer in a subject comprising administration of Compound D or a pharmaceutically acceptable salt thereof in combination or alternation with azacitidine to a host in need thereof. In another embodiment, provided is a method of treating tumor or cancer in a subject comprising administration of an analog of Compound A or a pharmaceutically acceptable salt thereof as provided herein in combination or alternation with azacitidine to a host in need thereof. Alternatively, provided is a method of treating tumor or cancer in a subject comprising administration of an analog of Compound B or a pharmaceutically acceptable salt thereof as provided herein in combination or alternation with azacitidine to a host in need thereof. Alternatively, provided is a method of treating tumor or cancer in a subject comprising administration of an analog of Compound C or a pharmaceutically acceptable salt thereof as provided herein in combination or alternation with azacitidine to a host in need thereof. Alternatively, provided is a method of treating tumor or cancer in a subject comprising administration of an analog of Compound D or a pharmaceutically acceptable salt thereof as provided herein in combination or alternation with azacitidine to a host in need thereof.

### VI. Illustrative Examples

### Example 1. General Routes of Synthesis

Compounds of the present invention can be made in a modular fashion from known intermediate 1 (WO 2015/100420).

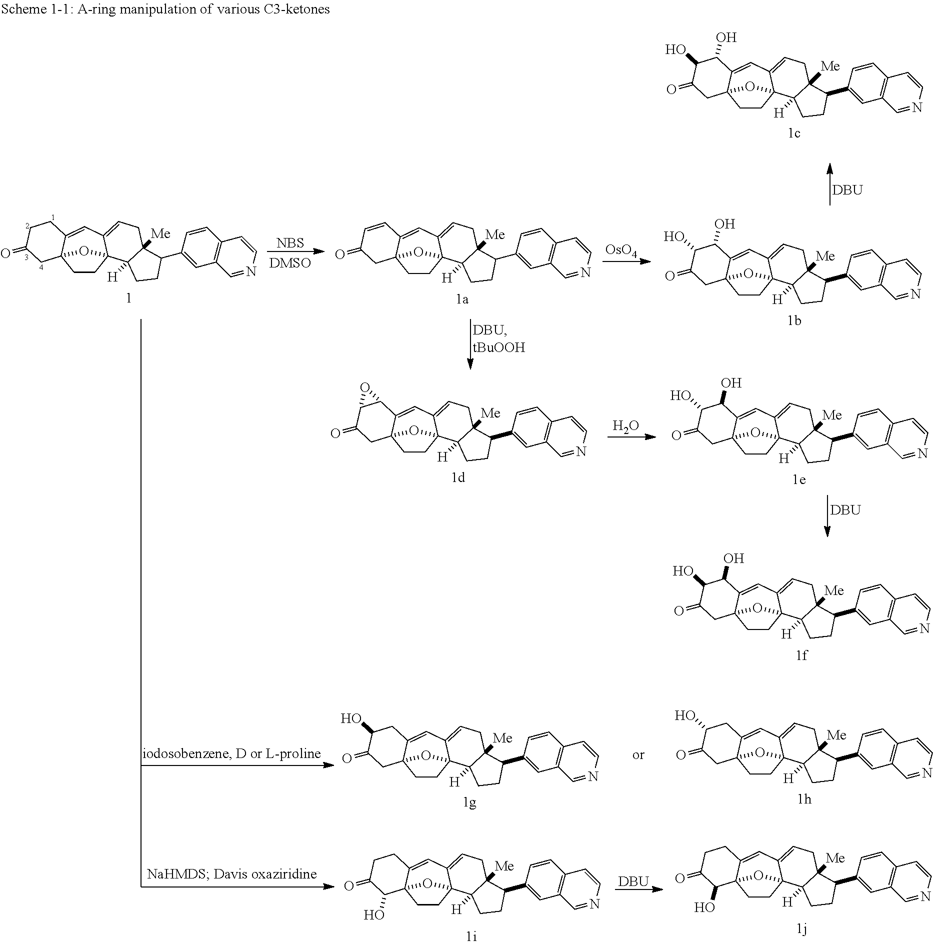

Known compound 1 undergoes in situ bromination/elimination to afford conjugated trienone 1a by treating with NBS in DMSO. Dihydroxylation conditions (for example, OsO₄) of 1a afford diol 1b. Basic conditions (for example, DBU) affect thermodynamic epimerization of C2-OH to give trans diol 1c. Meanwhile, trienone 1a is epoxidized (for example, tBuOOH/DBU) to form compound id, and the epoxide ring can be opened from the allylic C1 position in aqueous media to afford trans diol 1e. Again, thermodynamic epimerization affords the formation of cis diol 1f from 1e.

Selective alpha-hydroxylation proceeds in the presence of a chiral directing reagent. For example, C2-beta-OH 1g is selectively generated when compound 1 is treated with iodosobenzene in the presence of organocatalyst D-proline. In the same manner, L-proline affords C2-alpha-OH 1h. On the other hand, C4-alpha-OH 1i is selectively formed by deprotonation with NaHMDS followed by addition of Davis oxaziridine. Base catalyzed (for example, DBU) thermodynamic epimerization of ii affords C4-beta-OH 1j.

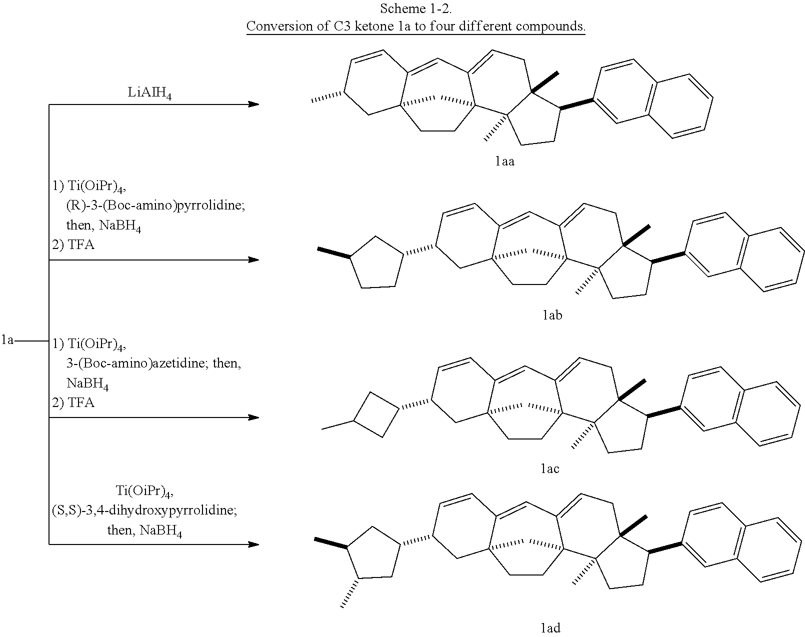

C3 ketone 1a diverges to four compounds as shown in Scheme 2. For example, reduction of ketone 1a with LiAlH₄ affords compound 1aa. Ti(OiPr)₄ assisted reductive amination (either with (R)-3-(Boc-amino)pyrrolidine or 3-(Boc-amino)azetidine) followed by TFA treatment to deprotect the Boc protecting group completes the synthesis of compound lab or lac. The same reductive amination condition can be conducted using (S,S)-3,4-dyhydroxypyrrolidine as an amine building block to afford compound lad.

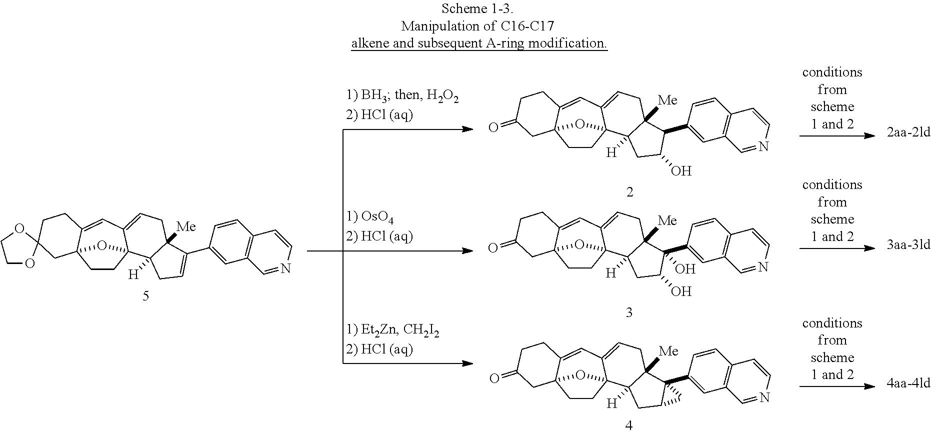

From the known compound 5, the C16-C17 double bond is modified in three different pathways (Scheme 3). Hydroboration (for example, BH3/H₂O₂) or dihydroxylation (for example, OsO₄), and subsequent ketal deprotection (for example, HCl) provides compounds 2 and 3 respectively. Meanwhile, Simmons-Smith conditions (for example, Et₂Zn/CH₂I₂) facilitate cyclopropanation that is followed bt ketal deprotection to afford compound 4. Again, the same conditions proposed in Schemes 1-1 and 1-2 can be applied to compounds 2, 3, and 4.

Non-limiting examples of compounds of the present invention that can be synthesized by the modular synthesis of Schemes 1-1, 1-2, and 1-3 include:

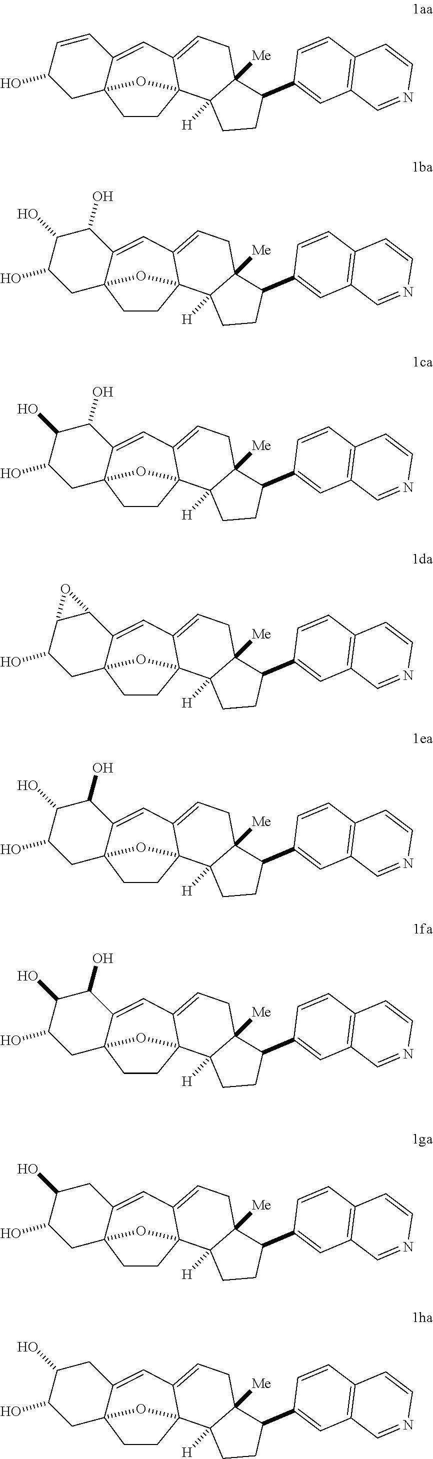

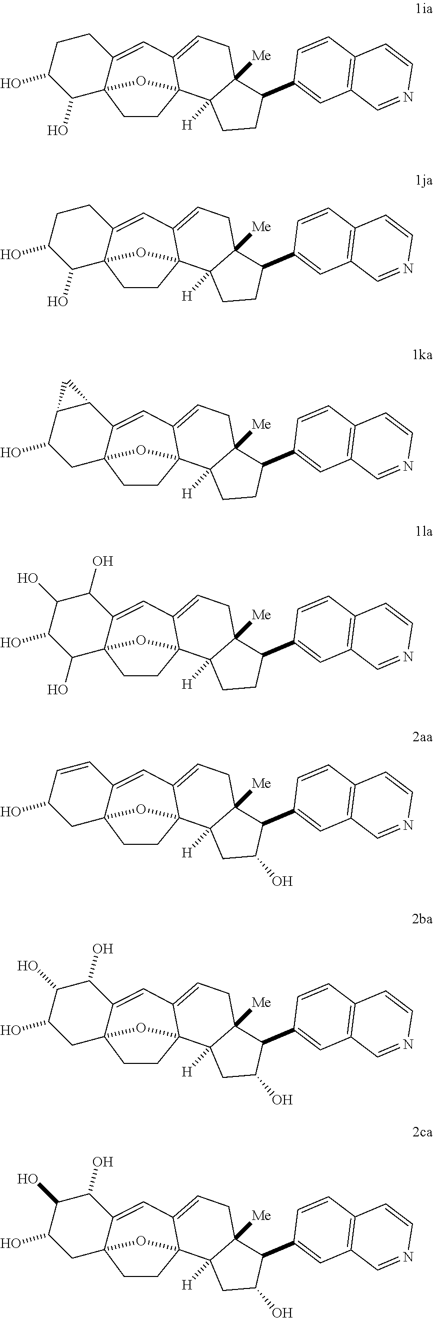

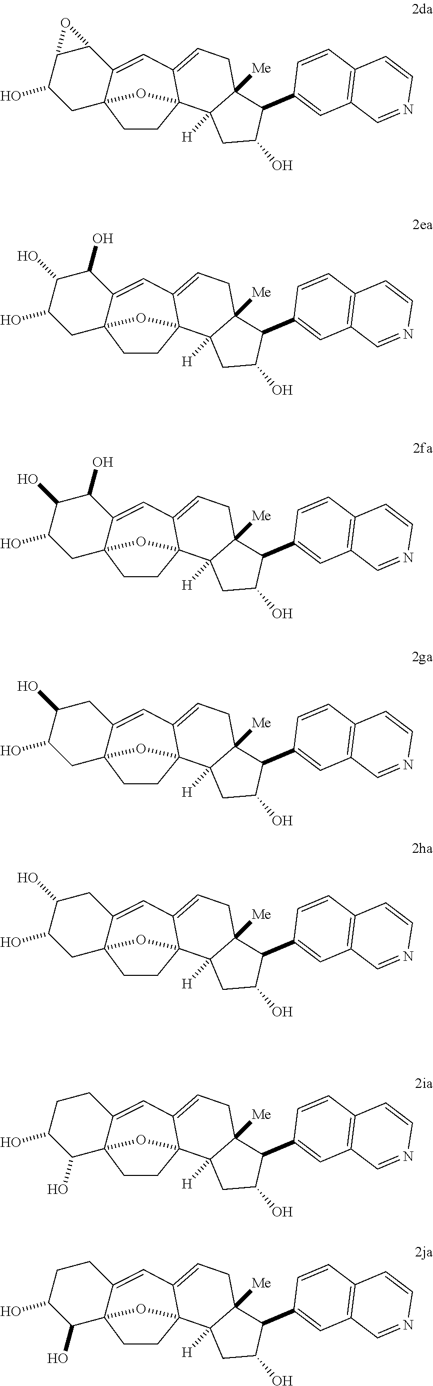

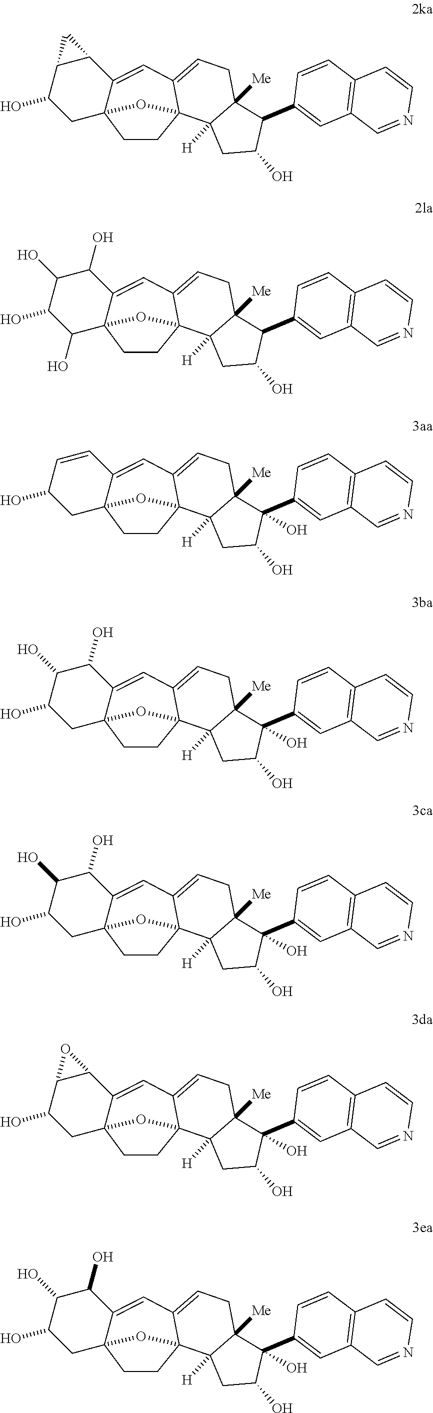

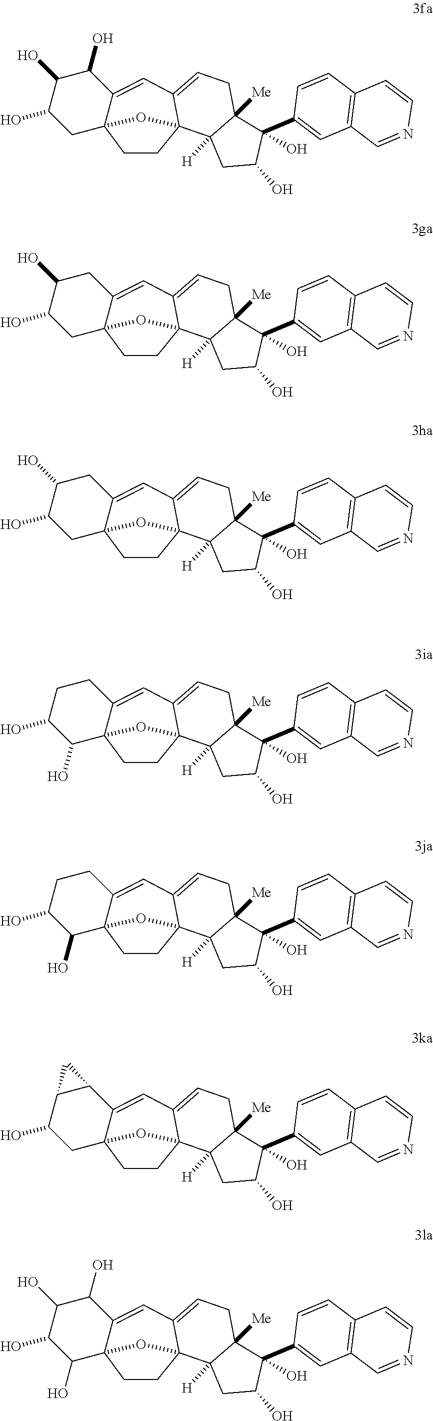

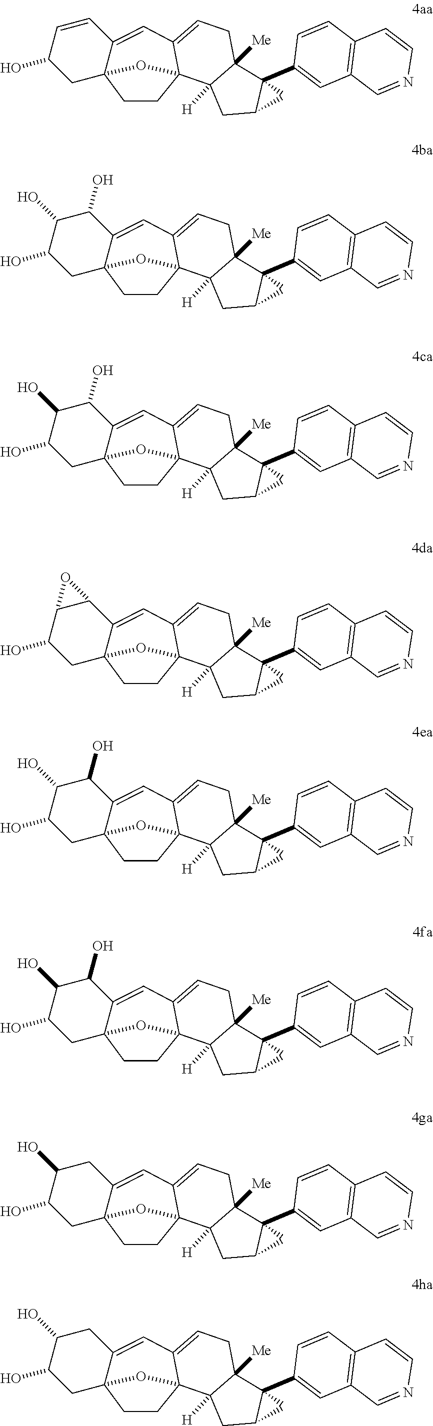

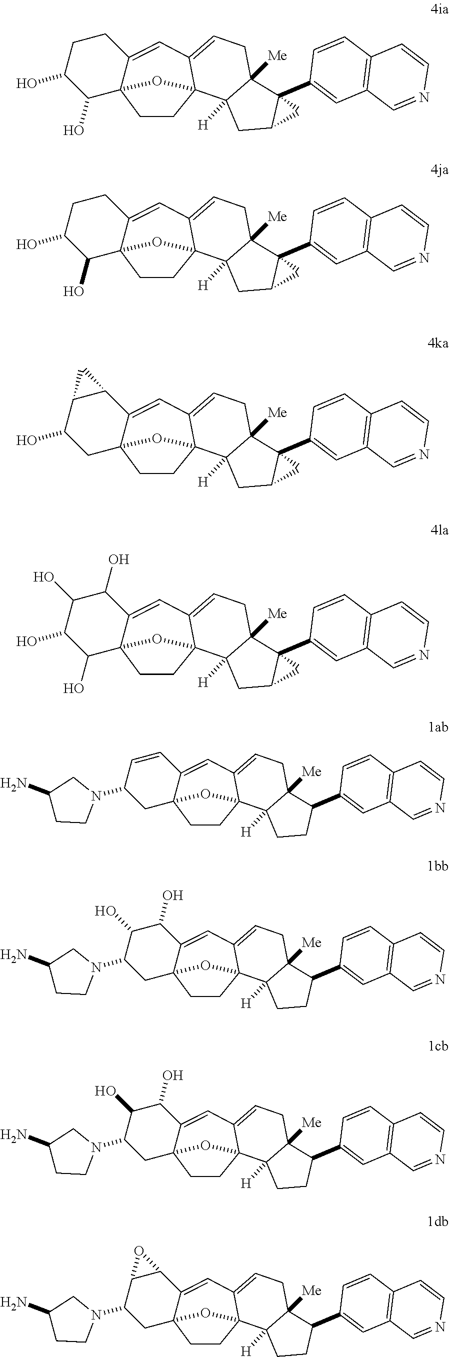

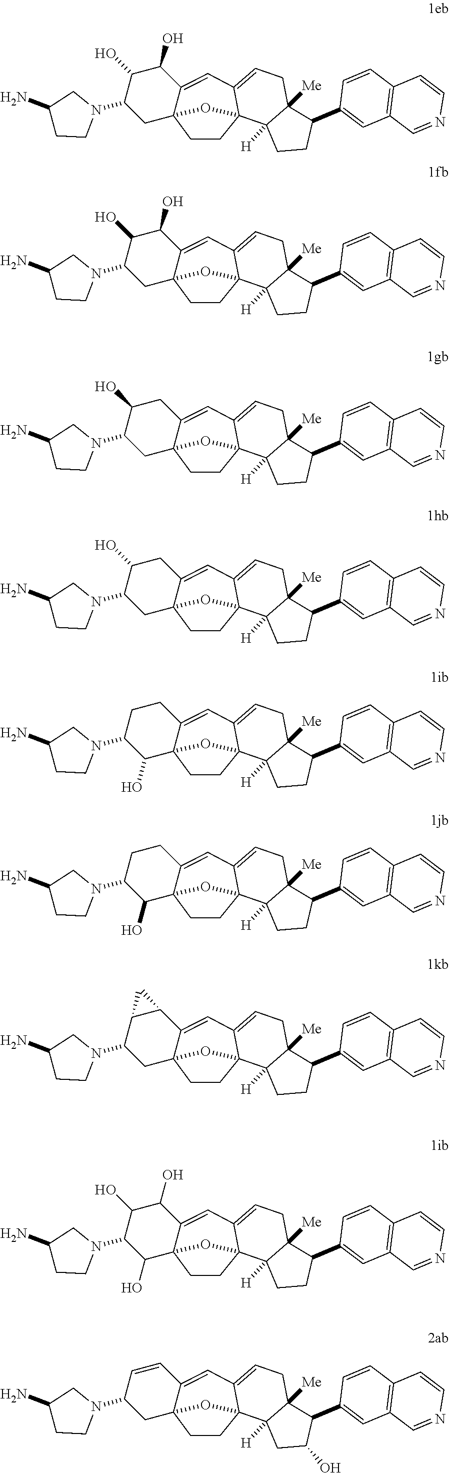

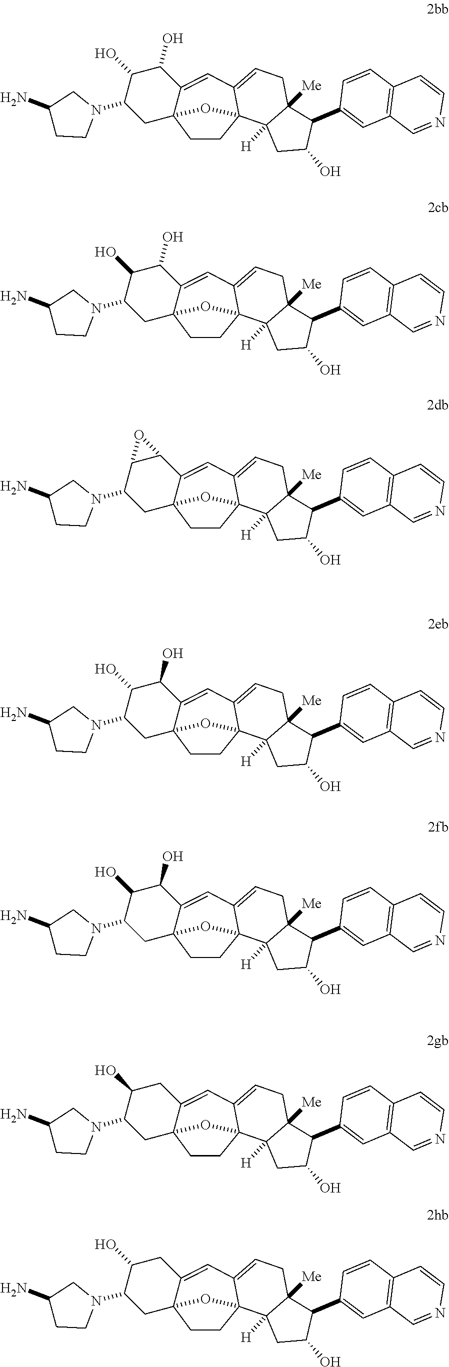

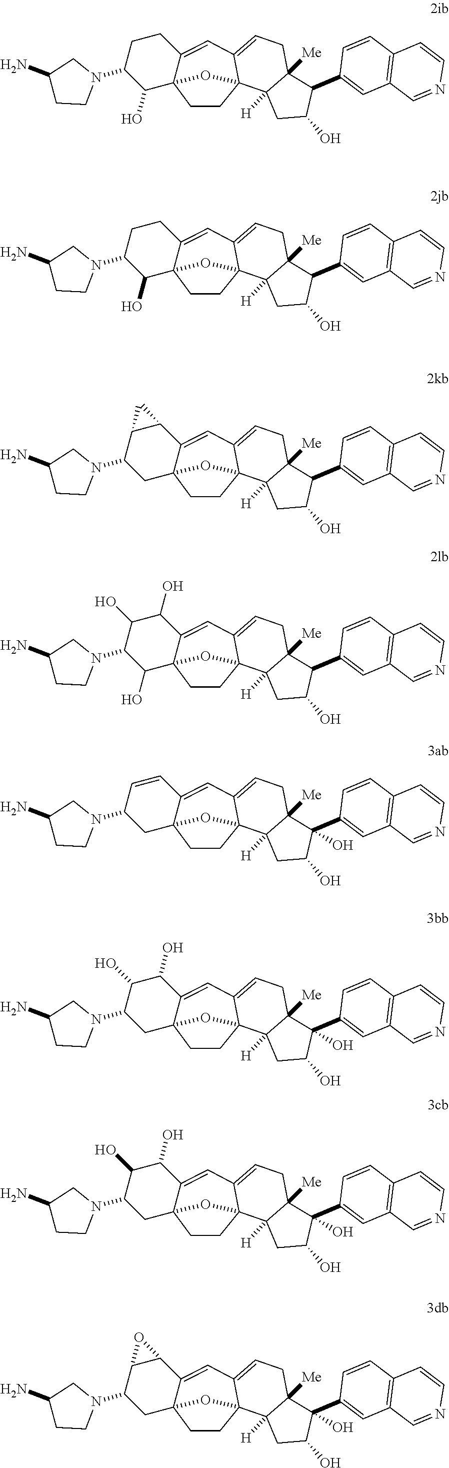

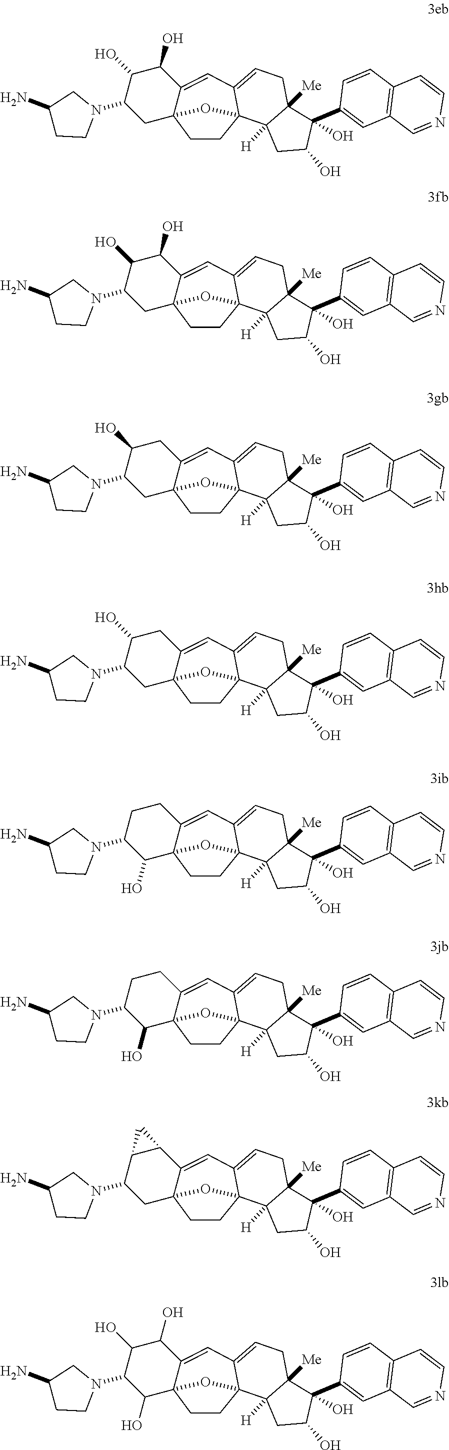

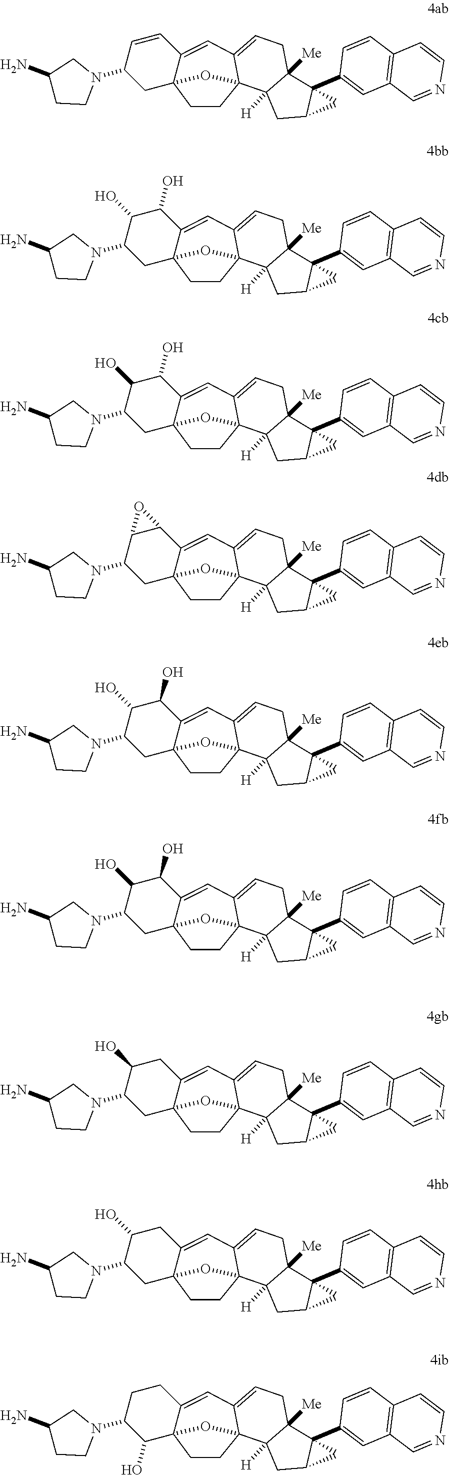

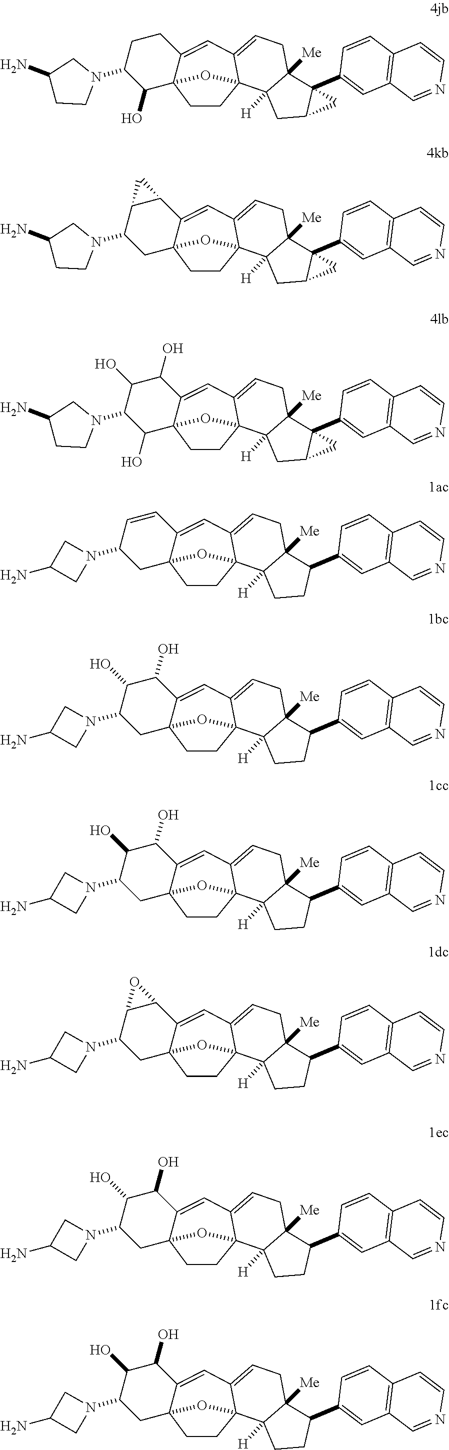

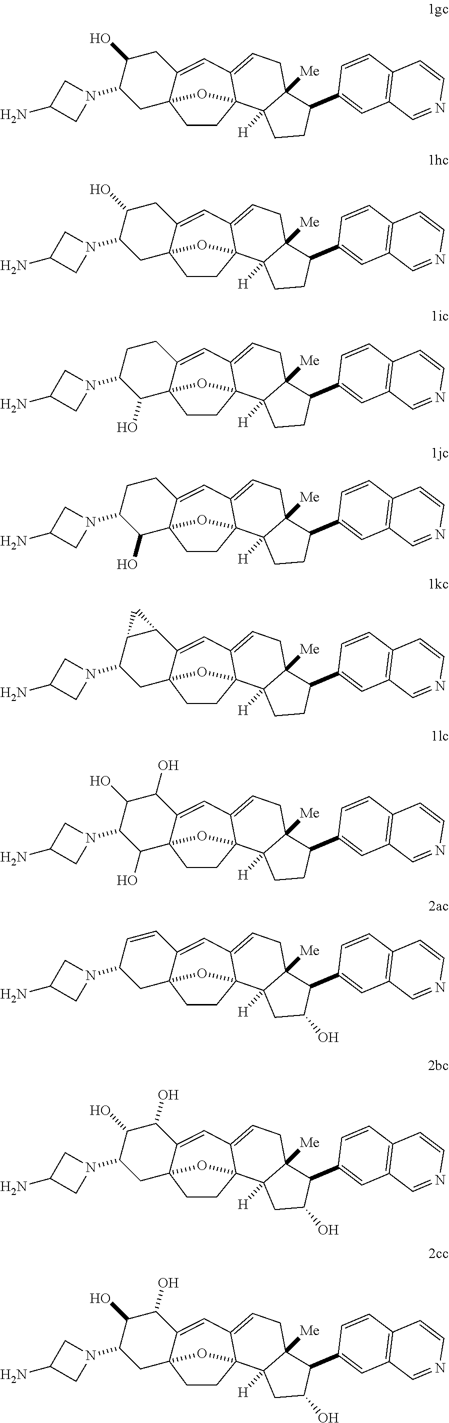

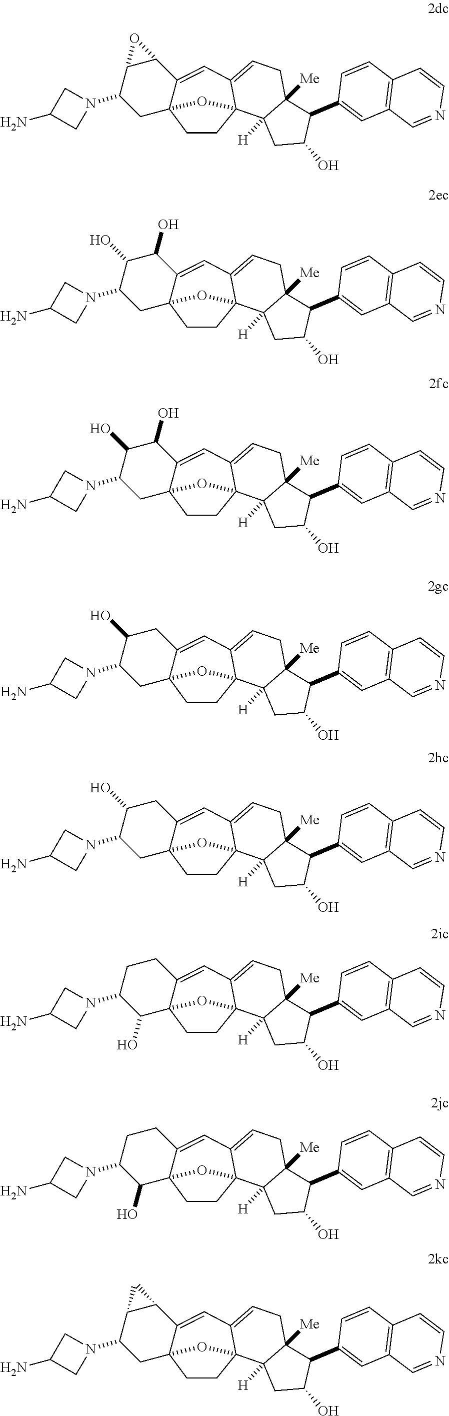

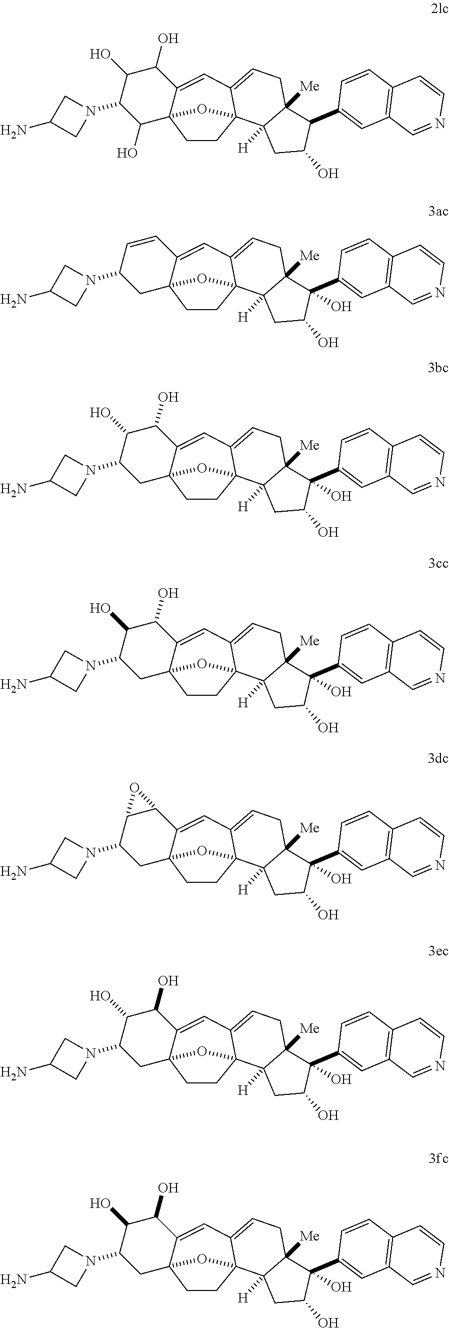

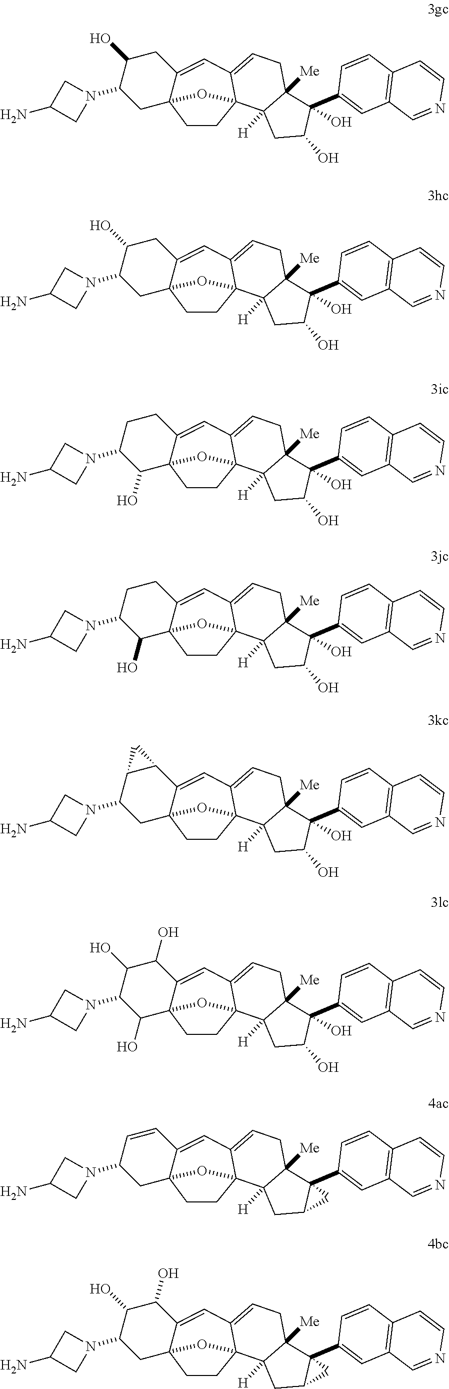

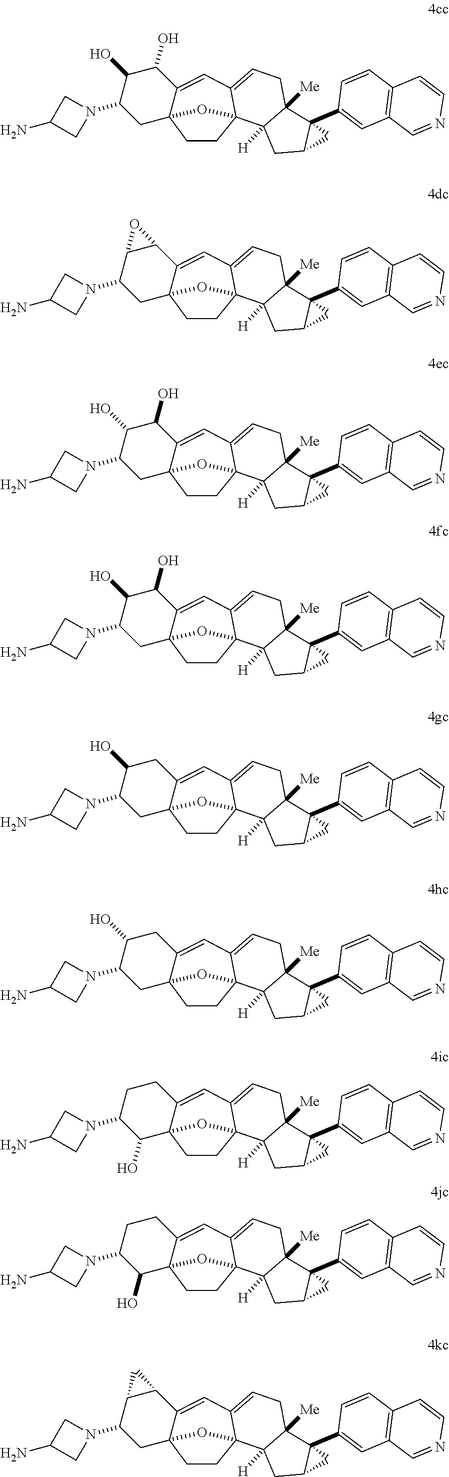

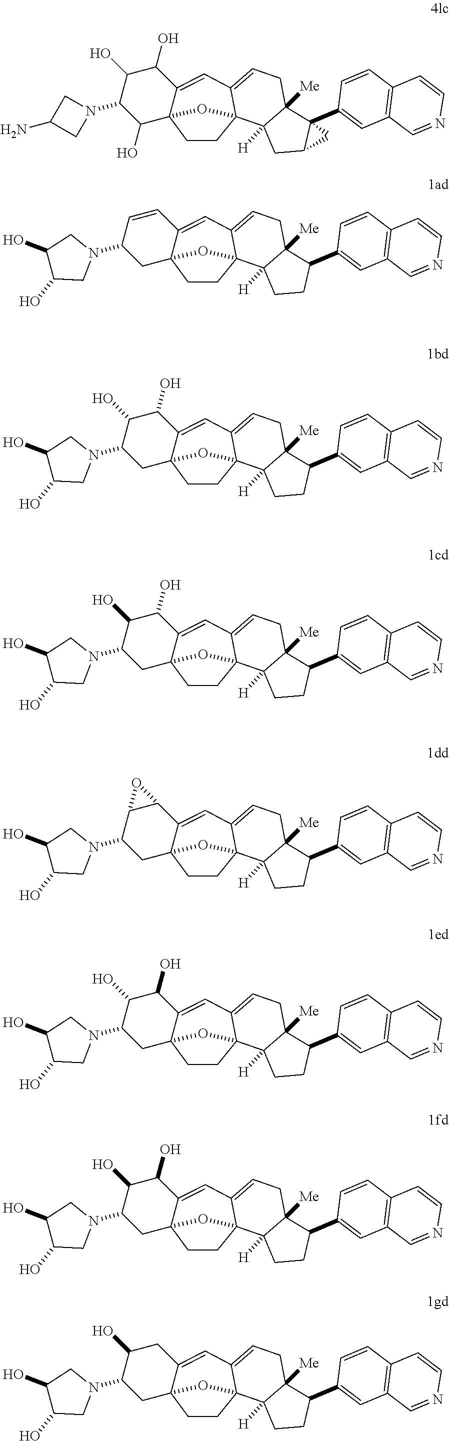

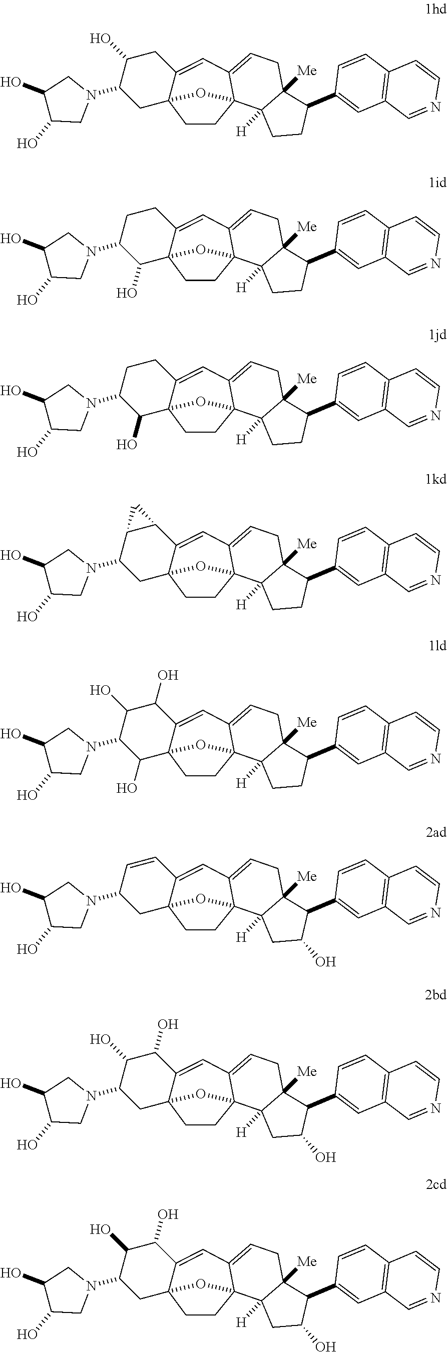

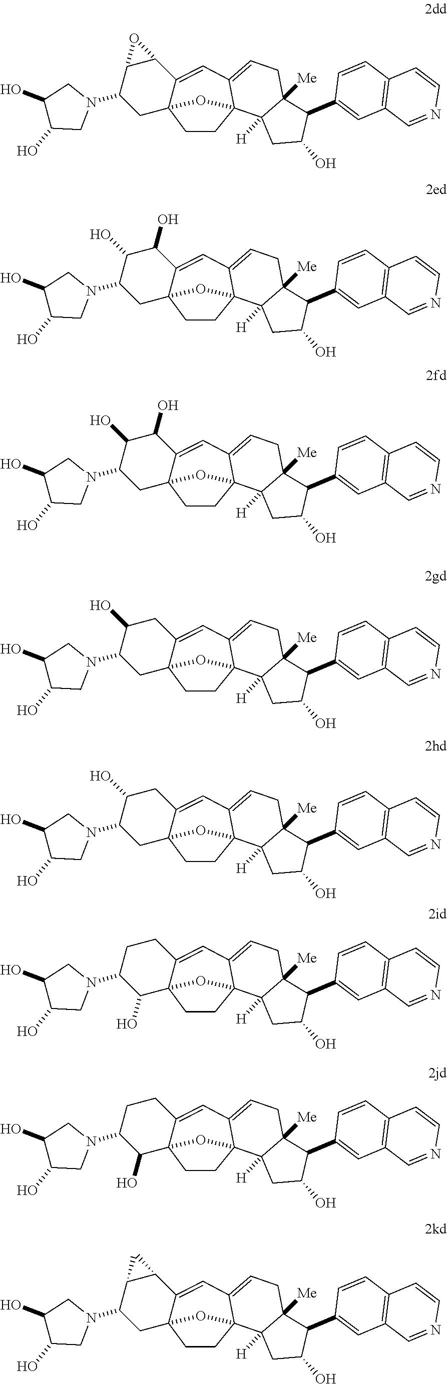

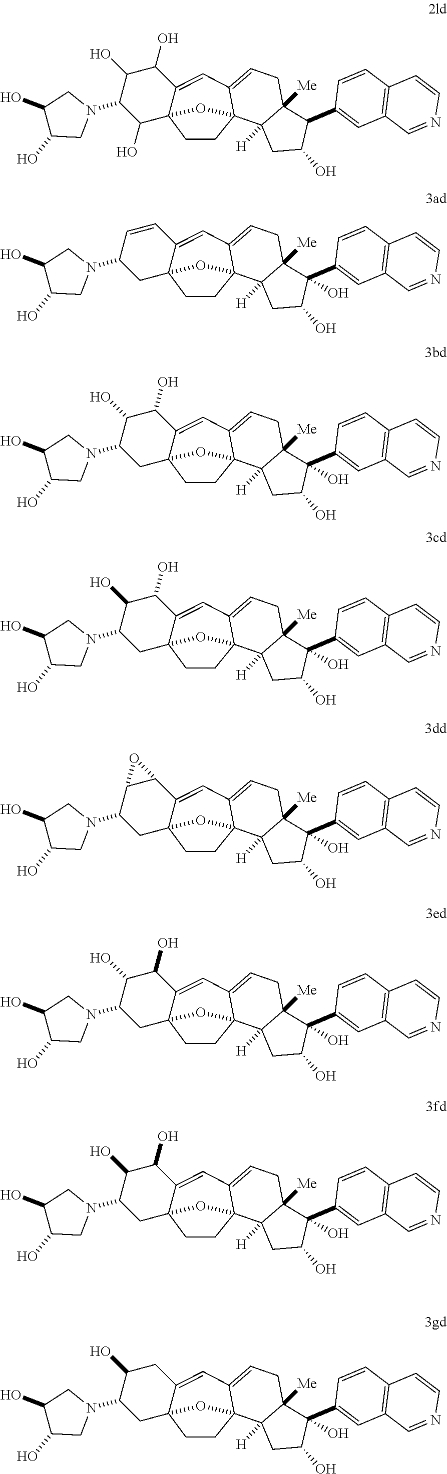

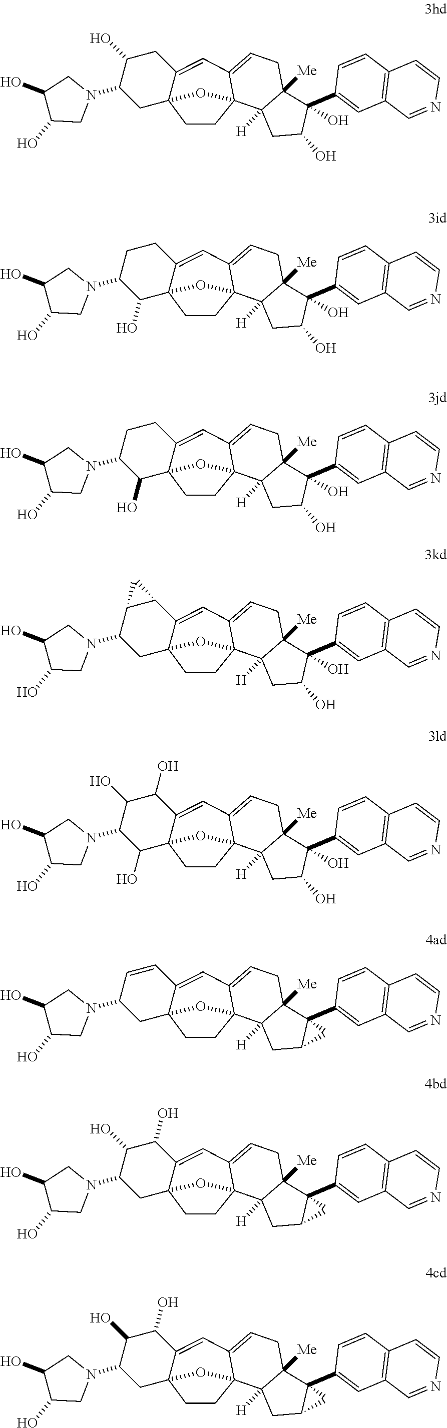

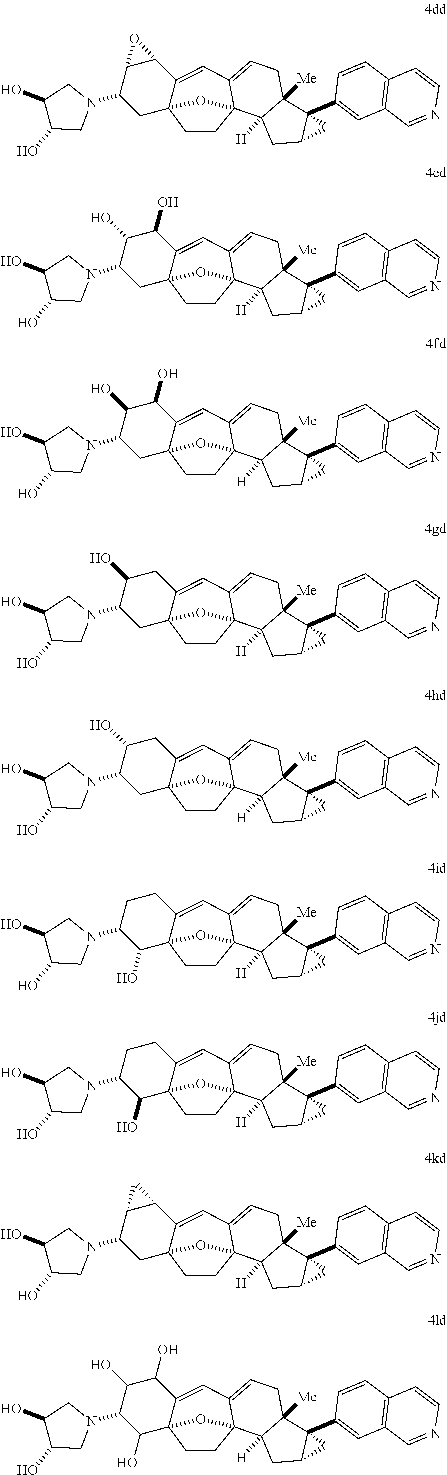

### Example 2. Representative Routes of Synthesis

### Abbreviations

### AIBN Azobisisobutyronitrile

### AUC Area Under the Curve

### DBU 1,8 Diazabicycloundec-7-ene

DCM, CH₂Cl₂ Dichloromethane
DDQ 2,3-Dichloro-5,6-dicyano-1,4-benzoquinone

### DMAP 4-Dimethylaminopyridine

### DMF N,N-dimethylformamide

DMSO Dimethyl sulfoxide
DTBMP 2,6-Di-tert-butyl-4-methylpyridine
ESI Electrospray ionization
EtOAc Ethyl acetate

### Et Ethyl

### h, hr Hour

### HPLC High Pressure Liquid Chromatography

### iPr Iso-propyl

K₂CO₃ Potassium carbonate
mCPBA meta-Chloroperoxybenzoic acid
MMPP Magnesium monoperoxyphthalate

### NBS N-Bromosuccinimide

### NMR Nuclear Magnetic Resonance

PTSA p-Toluenesulfonic acid
RT Room temperature

### TEA Trimethylamine

### tBu Tert-butyl

TFA Trifluoroacetic acid

### THF Tetrahydrofuran

### GENERAL METHODS

The structure of starting materials, intermediates, and final products was confirmed by standard analytical techniques, including NMR spectroscopy and mass spectrometry. Unless otherwise noted, reagents and solvents were used as received from commercial suppliers.

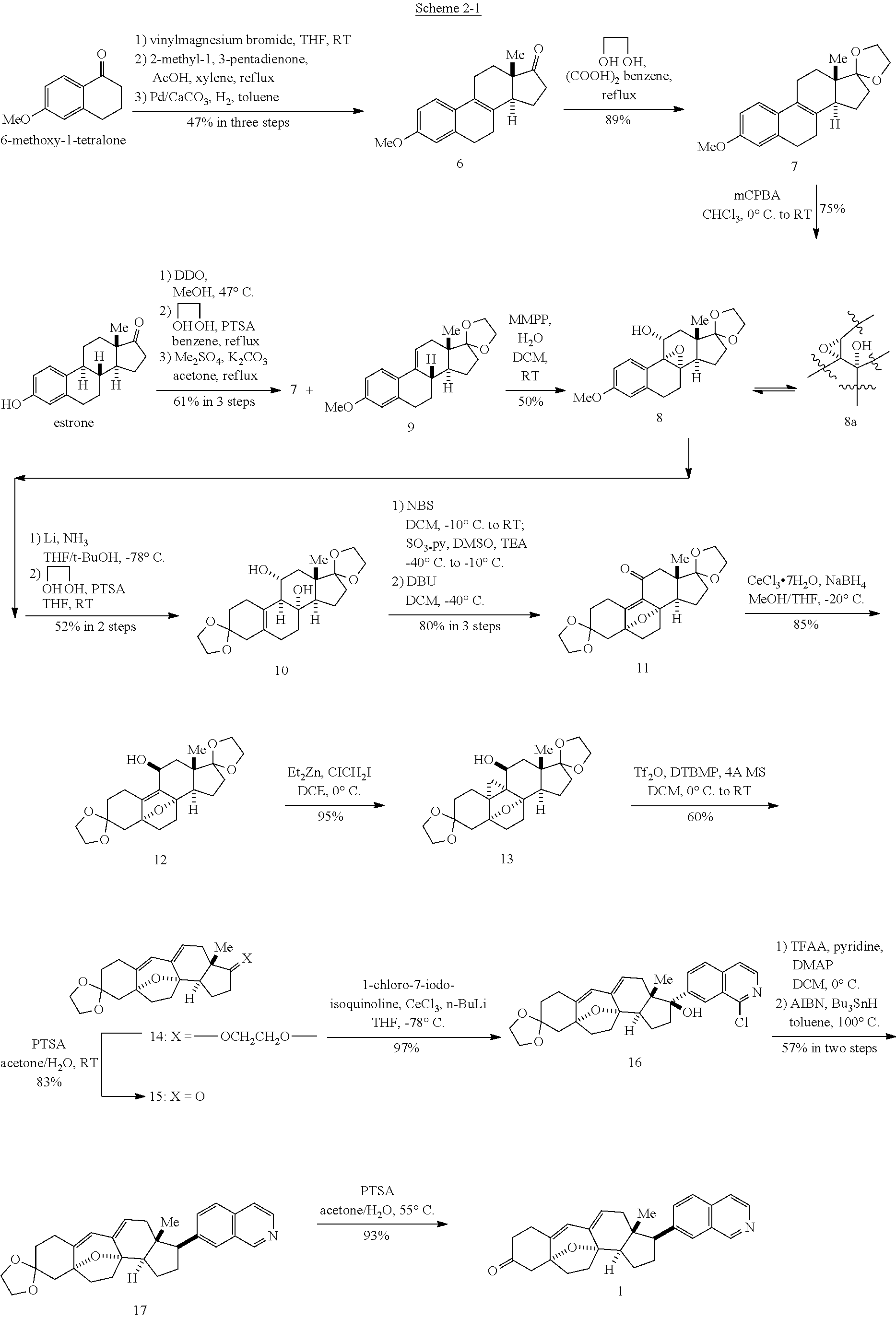

### Scheme 2-1: Synthesis of Ketone 1

### 8,9-Unsaturated Methoxyethyleneketone (Compound 6)

The Grignard reaction was performed with 20.0 g (113 mmol, 1.00 equiv) of 6-methoxy-1-tetralone and the product was carried forward without purification by flash chromatography. See, Saraber et al., Tetrahedron 2006, 62, 1726-1742. To a solution of Grignard reaction product and 2-methyl-1,3-pentadienone (12.8 g, 114 mmol, 1.01 equiv) in xylene (140 mL) was added AcOH (64.6 mL, 1.13 mol, 10.0 equiv) and the reaction mixture was warmed to reflux. After 2 hours, the reaction was allowed to cool to room temperature and concentrated under reduced pressure. A mixture toluene and ethyl ether (1:1) was added to dissolve the solid residue and the mixture was filtered. The filtrate was washed sequentially with saturated NaHCO₃ solution (200 mL) and brine, dried over MgSO₄, and concentrated under reduced pressure. The residue was purified by flash chromatography (silica gel, eluent: 20:1:1 hexanes:EtOAc:DCM) to afford the Torgov's diene. Spectral data was consistent with those previously reported. See Soorukram, D.; Knochel, P. Org. Lett. 2007, 9, 1021-1023. The Torgov's diene was converted to 8,9-unsaturated methoxyethyleneketone 6 (15.0 g, 47% over 3 steps) based on the known procedure from the literature. See, Sugahara et al., Tetrahedron Lett. 1996, 37, 7403-7406.

### 8,9-Unsaturated Methoxyethyleneketal (Compound 7)

To a solution of compound 6 (15.0 g, 53.1 mmol, 1.0 equiv) in benzene (215 mL) and ethylene glycol (72 mL) was added oxalic acid (2.30 g, 12.1 mmol, 0.22 equiv). The reaction mixture was allowed to warm to reflux and water was trapped by a Dean-Stark apparatus. After 16 hours, the reaction was cooled to room temperature and saturated NaHCO₃ solution (150 mL) was added. The organic and aqueous layers were separated and the aqueous phase was extracted with ethyl acetate (2×200 mL). The combined organic phases were washed with brine (150 mL) and dried over Na₂SO₄. The solvent was evaporated under reduced pressure and the residue was purified by flash chromatography (silica gel, eluent: 15:1 hexanes:EtOAc) to afford 8,9-unsaturated methoxyethyleneketal compound 7 (15.5 g, 89%).

¹H NMR (500 MHz, CDCl₃) δ=7.13 (d, J=8.3 Hz, 1H), 6.73-6.67 (m, 2H), 4.05-3.85 (m, 4H), 3.79 (s, 3H), 2.82-2.65 (m, 2H), 2.52-2.45 (m, 2H), 2.23-2.17 (m, 2H), 2.14 (ddd, J=2.2, 11.6, 14.0 Hz, 1H), 1.99-1.82 (m, 4H), 1.64 (td, J=4.2, 12.2 Hz, 1H), 1.49 (dq, J=6.8, 11.6 Hz, 1H), 0.86 (s, 3H). HRMS (ESI) (min) calc'd for C₂₁H₂₇O₃ [M+H]⁺: 327.1955, found 327.1947.

### Epoxy Alcohols 8 and 8a

A solution of 8,9-unsaturated ethyleneketal 7 (1.63 g, 5.00 mmol, 1.0 equiv) in CHCl₃ (50 mL) was cooled to 0° C. and mCPBA (77% max, 2.46 g, 11.0 mmol, 2.2 equiv) was added. The reaction mixture was stirred for 10 minutes at 0° C. and warmed to room temperature. After an additional 50 minutes, 10% Na₂S₂O₃ solution (40 mL) and saturated NaHCO₃ solution (40 mL) were added sequentially. The organic and aqueous layers were separated and the aqueous phase was extracted with dichloromethane (3×50 mL). The combined organic phases were washed with brine (50 mL), dried over Na₂SO₄, and concentrated under reduced pressure. The residue was purified by flash chromatography (silica gel, eluent: 3:1-1:1 hexanes:EtOAc) to afford epoxy alcohol 8 and 8a (1.40 g, 75%). 8 and 8a were under equilibration in any solvent, with a majority of the mixture existing as 8. H NMR was analyzed for epoxy alcohol 8.

¹H NMR (500 MHz, CDCl₃) δ=7.77 (d, J=8.3 Hz, 1H), 6.76 (dd, J=2.0, 8.3 Hz, 1H), 6.63 (d, J=2.0 Hz, 1H), 4.78 (dd, J=7.8, 9.8 Hz, 1H), 3.95-3.87 (m, 4H), 3.78 (s, 3H), 2.84 (dt, J=5.9, 14.4 Hz, 1H), 2.49 (dd, J=4.4, 15.1 Hz, 1H), 2.36-2.29 (m, 1H), 2.26 (dd, J=5.9, 14.2 Hz, 2H), 2.06 (t, J=11.7 Hz, 1H), 1.97 (dd, J=7.3, 12.2 Hz, 1H), 1.94-1.88 (m, 2H), 1.75 (dt, J=5.4, 14.2 Hz, 1H), 1.63-1.53 (m, 1H), 1.46 (t, J=11.0 Hz, 1H), 0.75 (s, 3H). HRMS (ESI) (m/z) calc'd for C₂₁H₂₇O₅ [M+H]⁺: 359.1853, found 359.1852.

### 8,9 and 9,11-Unsaturated Methoxyethyleneketal Compounds 7 and 9

The DDQ oxidation was done with 22.0 g (81.4 mmol, 1.0 equiv) of estrone and the product was carried forward without purification by flash chromatography. See, Stephan et al., Steroid. 1995, 60, 809-811. To a solution of 9,11-unsaturated estrone in benzene (375 mL) was added ethylene glycol (110 mL, 1.99 mol, 24.4 equiv) and PTSA (3.00 g, 16.3 mmol, 0.20 equiv). The reaction mixture was warmed to reflux and water was trapped by a Dean-Stark apparatus. After 18 hours, the reaction was allowed to cool to room temperature and saturated NaHCO₃ solution (300 mL) was applied. The aqueous phase was extracted with ethyl acetate (2×300 mL) and the combined organic phases were washed with brine (200 mL). The organic phase was dried (Na₂SO₄) and the solvent was evaporated under reduced pressure. The product was carried forward in the next step without further purification.

The ethyleneketal (mixture of the 8,9 and 9, 1-unsaturated regioisomers) was dissolved in acetone (420 mL) and K₂CO₃ (22.5 g, 163 mmol, 2.00 equiv) was added. This was followed by the addition of Me₂SO₄ (9.30 mL, 97.6 mmol, 1.20 equiv) and the reaction mixture was warmed to reflux. After 18 hours, the reaction was allowed to cool to room temperature and the acetone was evaporated. 2M NaOH solution was added (300 mL) and the aqueous phase was extracted with ethyl acetate (2×300 mL). The combined organic phases were dried (Na₂SO₄) and the solvent was evaporated under reduced pressure. The residue was purified by flash chromatography (silica gel, eluent: 15:1 hexanes:EtOAc) to afford a mixture of 8,9 and 9,11-unsaturated methoxyethyleneketal compounds 7 and 9 (16.3 g, 61% in three steps, ˜4:5 mixture of 8,9-unsaturated:9,11-unsaturated regioisomers).

For the 9,11-unsaturated isomer, only distinguishable peaks were assigned: ¹H NMR (500 MHz, CDCl₃) δ=7.53 (d, J=8.8 Hz, 1H), 6.60 (d, J=2.0 Hz, 1H), 6.13 (td, J=2.6, 5.0 Hz, 1H), 3.79 (s, 3H), 2.59 (td, J=3.2, 17.6 Hz, 1H), 2.09-2.00 (m, 3H), 1.45-1.33 (m, 2H), 0.90 (s, 3H). HRMS (ESI) (m/z) calc'd for C₂₁H₂₇O₃ [M+H]⁺: 327.1955, found 327.1951.

### Epoxy Alcohol Compounds 8 and 8a

To a solution of the mixture of 8,9 and 9,11-unsaturated ethyleneketal compounds 7 and 9 (15.7 g, 48.1 mmol, 1.00 equiv) in dichloromethane (700 mL) was added magnesium monoperoxyphthalate hexahydrate (68.4 g, 111 mmol, 2.30 equiv) and water (4.8 mL). The reaction mixture was stirred for 20 hours at room temperature and then quenched with the mixture of 10% aqueous Na₂S₂O₃ (300 mL) and saturated NaHCO₃ solution (300 mL). The organic and aqueous layers were separated and the aqueous phase was extracted with dichloromethane (2×500 mL). The combined organic phases were washed with brine (300 mL) and dried (Na₂SO₄). The solvent was evaporated under reduced pressure and the residue was purified by flash chromatography (silica gel, eluent: 3:1→2:1 hexanes:EtOAc) to provide epoxy alcohol 8 and 8a (8.60 g, 500/%). Spectral data was consistent with epoxy alcohol 8 and 8a constructed from 8,9-unsaturated methoxyethyleneketal 6.

### Diol Compound 10

Ammonia gas was condensed (240 mL) and to the liquid ammonia was added Li (3.90 g, 565 mmol, 25.0 equiv) at −78° C. After stirring for 30 minutes, epoxy alcohol 8 and 8a (8.10 g, 22.6 mmol, 1.0 equiv) in THF (110 mL) was cannulated and stirred for an additional 1.5 hours at that temperature. To the reaction mixture was added the mixture of t-BuOH (32 mL) and THF (16 mL) at −78° C. and the reaction stirred for an additional 20 minutes at that temperature. The mixture of I-BuOH (92 mL) and THF (38 mL) was added followed by benzene (50 mL) and water (50 mL) at −78° C., and the flask was opened to gently evaporate liquid ammonia by removing the cooling bath. Water (200 mL) was added and the aqueous phase was extracted with ethyl acetate (2×250 mL). The combined organic phases were washed with brine (150 mL), dried (Na₂SO₄), and concentrated under reduced pressure. The product was used in the next step without further purification.

To a solution of Birch reduction product in THF (300 mL) and ethylene glycol (75 mL) was added PTSA (430 mg, 2.26 mmol, 0.10 equiv). The reaction mixture was stirred for 30 minutes at room temperature and saturated NaHCO₃ solution (200 mL) was added. The organic and aqueous layers were separated and the aqueous phase was extracted with ethyl acetate (4×250 mL). The combined organic phases were washed with brine (200 mL) and dried (Na₂SO₄). The solvent was evaporated under reduced pressure and the residue was purified by flash chromatography (silica gel, eluent: 4:1 hexanes:EtOAc→1000% EtOAc→10:1 EtOAc:MeOH) to provide diol 10 (4.60 g, 52%).

¹H NMR (500 MHz, C₆D₆) δ=3.67-3.42 (m, 9H), 3.25-3.14 (m, 1H), 2.40 (dd, J=5.9, 13.2 Hz, 1H), 2.31 (br. s, 2H), 2.23-2.09 (m, 2H), 2.03 (t, J=10.7 Hz, 1H), 1.97-1.90 (m, 2H), 1.89 (dd, J=8.3, 14.2 Hz, 1H), 1.85-1.75 (m, 4H), 1.66-1.50 (m, 4H), 1.00 (s, 3H). HRMS (ESI) (m/z) calc'd for C₂₂H₃₂NaO₆ [M+Na]⁺: 415.2091, found 415.2076.

### Scheme 2-1: Enone Compound 11

To a solution of diol 10 (4.05 g, 10.3 mmol, 1.00 equiv) in dichloromethane (230 mL) was added NBS (2.00 g, 11.4 mmol, 1.10 equiv) in one portion at −10° C. and the reaction mixture was warmed to room temperature. The reaction was monitored by TLC (about 30 minutes for completion). Once the reaction was done, the reaction mixture was cooled to −40° C. and triethylamine (17.3 mL, 124 mmol, 12.0 equiv) was added. SO₃-Py (16.4 g, 103 mmol, 10.0 equiv) in DMSO (115 mL) was pre-stirred for 20 minutes at room temperature and was added to the reaction mixture at ˜40° C., which was subsequently allowed to warm slowly to −10° C. After 4 hours, saturated NH₄Cl solution (130 mL) was added and the reaction was allowed to warm to room temperature. The organic and aqueous layers were separated and the aqueous phase was extracted with dichloromethane (2×200 mL). The combined organic phases were washed with brine (150 mL), dried over Na₂SO₄, and concentrated under reduced pressure. The product was carried forward without further purification.

The oxidation product was dissolved in dichloromethane (300 mL) and the reaction mixture was cooled to −40° C. followed by the slow addition of DBU (3.90 mL, 25.6 mmol, 2.50 equiv). After 15 minutes, saturated NH₄Cl solution (130 mL) was added and the reaction was allowed to warm to room temperature. The organic and aqueous layers were separated and the aqueous phase was extracted with dichloromethane (2×200 mL). The combined organic phases were washed with brine (150 mL), dried over Na₂SO₄, and concentrated under reduced pressure.

The residue was purified by flash chromatography (silica gel, eluent: 3:1-1:1 hexanes:EtOAc) to afford enone 11 (3.16 g, 80% in three steps).

¹H NMR (500 MHz, C₆D₆) δ=3.58-3.51 (m, 1H), 3.49-3.34 (m, 6H), 3.28-3.23 (m, 2H), 3.19 (dt, J=4.2, 7.7 Hz, 1H), 2.80 (d, J=16.1 Hz, 1H), 2.60 (ddd, J=6.8, 12.7, 19.0 Hz, 1H), 2.55 (d, J=13.2 Hz, 1H), 2.43 (d, J=16.1 Hz, 1H), 2.31 (dd, J=1.5, 13.2 Hz, 1H), 1.98-1.88 (m, 2H), 1.88-1.80 (m, 3H), 1.71 (ddd, J=4.2, 9.6, 11.6 Hz, 1H), 1.68-1.59 (m, 3H), 1.20 (ddd, J=3.7, 8.4, 11.4 Hz, 1H), 0.90 (s, 3H). HRMS (ESI) (m/z) calc'd for C₂₂H₂₈NaO₆ [M+Na]⁺: 411.1778, found 411.1786.

### Allylic Alcohol Compound 12

To a solution of enone 11 (3.20 g, 8.32 mmol, 1.00 equiv) in MeOH (150 mL) and THF (20 mL) was added CeCl₃.7H₂O (9.20 g, 24.7 mmol, 3.00 equiv) at room temperature. After stirring for 5 minutes, the reaction was cooled to −20° C. followed by the addition of NaBH₄ (623 mg, 16.5 mmol, 2.00 equiv). After 30 minutes, saturated NH₄Cl solution (50 mL) and water (50 mL) were added and the reaction was allowed to warm to room temperature. The aqueous phase was extracted with ethyl acetate (3×200 mL) and the combined organic phases were washed with brine (150 mL), dried over Na₂SO₄, and concentrated under reduced pressure. The residue was purified by flash chromatography (silica gel, eluent: 20:1 DCM:MeOH) to afford allylic alcohol 12 (2.72 g, 85%).

¹H NMR (500 MHz, C₆D₆) δ=4.39-4.30 (m, 1H), 3.58-3.36 (m, 8H), 3.22 (dd, J=3.7, 16.4 Hz, 1H), 2.94 (dd, J=7.1, 12.5 Hz, 1H), 2.66 (d, J=13.2 Hz, 1H), 2.49-2.41 (m, 1H), 2.39 (dd, J=2.2, 12.9 Hz, 1H), 2.07-1.99 (m, 1H), 1.96-1.79 (m, 6H), 1.73 (br. s, 3H), 1.66-1.57 (m, 1H), 1.15-1.07 (m, 1H), 0.86 (s, 3H). HRMS (ESI) (m/z) calc'd for C₂₂H₃₀NaO₆ [M+Na]⁺: 413.1935, found 413.1942.

Cyclopropane Compound 13 To a solution of ClCH₂I (1.98 mL, 27.1 mmol, 4.00 equiv) in 1,2-dichloroethane (140 mL) was added a solution of Et₂Zn in diethyl ether (1M, 13.6 mL, 13.6 mmol, 2.00 equiv) at 0° C. After stirring for 5 minutes, allylic alcohol 12 (2.65 g, 6.79 mmol, 1.00 equiv) in 1,2-dichloroethane (70 mL) was added to the reaction flask at 0° C. After 30 minutes, the reaction was quenched by saturated NH₄Cl solution (100 mL) and allowed to warm to room temperature. The organic and aqueous layers were separated and the aqueous phase was extracted with dichloromethane (2×120 mL). The combined organic phases were washed with brine (100 mL), dried over Na₂SO₄, and concentrated under reduced pressure. The residue was purified by flash chromatography (silica gel, eluent: 2:1-+1:1 hexanes:EtOAc) to afford cyclopropane 13 (2.59 g, 93%).

¹H NMR (500 MHz, C₆D₆) δ=3.92 (dd, J=3.7, 11.0 Hz, 1H), 3.51-3.40 (m, 8H), 2.72 (dd, J=7.1, 12.9 Hz, 1H), 2.39 (dd, J=5.4, 17.6 Hz, 1H), 2.38 (d, J=12.2 Hz, 1H), 2.15 (d, J=12.2 Hz, 1H), 2.12 (dt, J=4.9, 12.2 Hz, 1H), 2.02 (ddd, J=2.9, 11.2, 14.6 Hz, 1H), 1.92-1.82 (m, 3H), 1.82-1.73 (m, 2H), 1.69-1.54 (m, 5H), 1.52 (dd, J=6.1, 12.0 Hz, 1H), 1.49-1.44 (m, 1H), 0.98 (s, 3H), 0.86 (d, J=2.4 Hz, 1H), 0.15 (d, J=2.9 Hz, 1H). HRMS (ESI) (m/z) calc'd for C₂₃H₃₂NaO₆ [M+Na]⁺: 427.2091, found 427.2088.

### Oxabicyclo[3.2.1]Octane Compound 14

Cyclopropane 13 (2.45 g, 6.06 mmol, 1.00 equiv) and 2,6-di-tert-butyl-4-methylpyridine (4.40 g, 21.2 mmol, 3.50 equiv) were azeotropically dried with benzene and dissolved in dichloromethane (120 mL). 4 Å molecular sieves (3.1 g) were added and the reaction flask was cooled to 0° C. A solution of triflic anhydride in dichloromethane (1 M, 12.1 mL, 12.1 mmol 2.00 equiv) was added dropwise and the ice bath was removed to warm the reaction flask to room temperature. After 2 hours, the reaction was quenched with triethylamine (20 mL) and filtered through a pad of Celite. Saturated NaHCO₃ solution (100 mL) was added and the aqueous phase was extracted with dichloromethane (2×120 mL). The combined organic phases were washed with brine (100 mL), dried over Na₂SO₄, and concentrated under reduced pressure. The residue was purified by flash chromatography (silica gel, eluent: 3:1 pentane:diethyl ether) to afford oxabicyclo[3.2.1]octane compound 14 (1.42 g, 60%). See Magnus et al., Org. Lett. 2009, 11, 3938-3941.

¹H NMR (500 MHz, CDCl₃) δ=5.73 (s, 1H), 5.29-5.26 (m, 1H), 4.04-3.76 (m, 8H), 2.58-2.50 (m, 1H), 2.46 (t, J=15.1 Hz, 1H), 2.31-2.24 (m, 2H), 2.19 (t, J=11.2 Hz, 1H), 2.09 (d, J=13.2 Hz, 1H), 1.99 (dt, J=4.4, 13.2 Hz, 1H), 1.94 (dd, J=2.4, 13.2 Hz, 1H), 1.91-1.84 (m, 1H), 1.83-1.71 (m, 3H), 1.71-1.53 (m, 5H), 0.88 (s, 3H). HRMS (ESI) (m/z) calc'd for C₂₃H₃₀O₅ [M+H]⁺: 387.2166, found 387.2180.

### Monoketone Compound 15

To a solution of bisethyleneketal 14 (110 mg, 285 μmol, 1.0 equiv) in acetone (14.6 mL) and water (3.6 mL) was added PTSA (21.6 mg, 85.2 μmol, 0.30 equiv) and the reaction mixture was stirred for 3 days. Saturated NaHCO₃ solution (5 mL) and ethyl acetate (25 mL) were sequentially added to the reaction. The layers were separated and the aqueous layer was extracted with ethyl acetate (2×15 mL). The organic layers were combined, washed with brine (20 mL), dried over Na₂SO₄ and concentrated under reduced pressure. The resulting residue was then purified by flash chromatography (silica gel, eluent: 4:1 hexanes:EtOAc) to afford monoketone 15 (79.0 mg, 81%).

¹H NMR (500 MHz, CDCl₃) δ=5.73 (s, 1H), 5.29-5.25 (m, 1H), 3.98-3.90 (m, 4H), 2.48 (dd, J=8.8, 19.5 Hz, 1H), 2.46-2.40 (m, 1H), 2.36 (dd, J=5.9, 12.7 Hz, 1H), 2.34-2.25 (m, 2H), 2.24-2.08 (m, 5H), 2.09 (d, J=13.2 Hz, 1H), 1.95 (dd, J=2.4, 13.2 Hz, 1H), 1.90-1.81 (m, 1H), 1.79-1.70 (m, 2H), 1.70-1.61 (m, 2H), 0.89 (s, 3H). HRMS (ESI) (m/z) calc'd for C₂₁H₂₇O₄ [M+H]⁺: 343.1909, found 343.1919.

### 1-Chloroisoquinoline Adduct Compound 16

CeCl₃ (565 mg, 2.30 mmol, 10.0 equiv) in a reaction flask was heated at 140° C. under vacuum for 2 hours. The flask was charged with Ar and cooled to 0° C. After 30 minutes, THF (2.8 mL) was added and stirred at 0° C. for 2 hours. The flask was then allowed to warm to room temperature and stirred for additional 16 hours.

1-Chloro-7-iodoisoquinoline was synthesized following the procedure provided in Subasinghe et al., Bioorg. Med. Chem. Lett. 2013, 23, 1063-1069.

To a solution of CeCl₃/THF complex was added 1-chloro-7-iodoisoquinoline (396 mg, 1.40 mmol, 6.00 equiv) in THF (1.4 mL). The reaction was stirred for 10 minutes at room temperature and then allowed to cool to −78° C. A solution of n-butyllithium in hexanes (1.6 M, 716 μL, 1.10 mmol, 5.00 equiv) was then added dropwise. The reaction mixture was stirred for an additional 30 minutes at the same temperature and monoketone 15 (78.5 mg, 229 μmol, 1.00 equiv) was cannulated in THF (1.4 mL). After an additional 30 minutes, saturated NH₄Cl solution (5 mL) was added to the stirred reaction mixture, which was then allowed to warm to room temperature. The mixture was diluted with EtOAc (5 mL) and the layers were separated. The aqueous layer was extracted with EtOAc (3×5 mL) and the organic layers were combined, washed with brine (5 mL), and dried over Na₂SO₄ and concentrated under reduced pressure. The resulting residue was then purified by flash chromatography (silica gel, eluent: 2:1 hexanes:EtOAc) to provide 1-chloroisoquinoline adduct 16 (115 mg, 97%).

¹H NMR (500 MHz, CDCl₃) δ=8.34 (br. s, 1H), 8.24 (d, J=5.9 Hz, 1H), 7.89-7.83 (m, 1H), 7.76 (d, J=8.3 Hz, 1H), 7.56 (d, J=5.9 Hz, 1H), 5.63 (s, 1H), 5.16-4.99 (m, 1H), 4.02-3.87 (m, 4H), 2.62 (ddd, J=4.4, 9.8, 14.2 Hz, 1H), 2.48-2.38 (m, 2H), 2.36-2.26 (m, 3H), 2.26-2.19 (m, 1H), 2.18-2.08 (m, 2H), 1.96 (dd, J=2.4, 13.7 Hz, 1H), 1.88 (dd, J=5.1, 17.8 Hz, 1H), 1.82-1.70 (m, 2H), 1.67-1.57 (m, 3H), 1.49 (d, J=17.6 Hz, 1H), 1.20-1.08 (m, 3H). HRMS (ESI) (m/z) calc'd for C₃₀H₃₂NaO₄NCl [M+Na]³⁰: 528.1918, found 528.1929.

### Isoquinoline Compound 17

A solution of 1-chloroisoquinoline adduct 16 (115 mg, 227 μmol, 1.00 equiv) in dichloromethane (20 mL) was cooled to 0° C. Pyridine (183 μL, 2.30 mmol, 10.0 equiv) and DMAP (13.9 mg, 114 μmol, 0.50 equiv) were then added sequentially to the solution. After 5 minutes, trifluoroacetic anhydride (158 μL, 1.14 mmol, 5.00 equiv) was added dropwise and stirred additional for an 30 minutes, at which point pH 7 phosphate buffer (15 mL) was added followed by warming the reaction flask to room temperature. The organic and aqueous layers were separated and the aqueous layer was extracted with dichloromethane (2×15 mL). The organic layers were combined, washed with brine (25 mL), dried over Na₂SO₄, and concentrated under reduced pressure. The resulting residue was then purified by short flash column chromatography (silica gel, eluent: 2:1 hexanes:EtOAc) to afford trifluoroacetylated product which was quickly used for the next step.

Trifluoroacetylated product (130 mg, 216 mmol, 1.00 equiv) was azeotropically dried with benzene and dissolved in benzene (4.3 mL). AIBN (106 mg, 647 μmol, 3.00 equiv) was added and the reaction flask was degassed by the freeze-pump thaw process (3 cycles). Bu₃SnH (1.16 mL, 4.31 mmol, 20.0 equiv) was added and the reaction mixture was allowed to warm to reflux. After 3 hours, the reaction mixture was cooled to room temperature and concentrated under reduced pressure. The resulting residue was then purified by flash column chromatography (silica gel, eluent: 4:1→3:1→1:1 hexanes:EtOAc) to afford isoquinoline 17 (67.0 mg, 65% in two steps). See also Yamashita et al., J. Org. Chem. 2011, 76, 2408-2425.

¹H NMR (500 MHz, CDCl₃) δ=9.21 (s, 1H), 8.46 (d, J=5.9 Hz, 1H), 7.77 (s, 1H), 7.73 (d, J=8.3 Hz, 1H), 7.61 (d, J=5.9 Hz, 1H), 7.57 (d, J=8.3 Hz, 1H), 5.74 (s, 1H), 5.29-5.23 (m, 1H), 4.00-3.90 (m, 4H), 3.11 (t, J=10.0 Hz, 1H), 2.49 (dd, J=8.3, 11.2 Hz, 1H), 2.47-2.41 (m, 1H), 2.38-2.24 (m, 4H), 2.24-2.14 (m, 2H), 2.12 (d, J=13.2 Hz, 1H), 2.06-1.95 (m, 2H), 1.91 (dd, J=5.4, 17.6 Hz, 1H), 1.83 (dq, J=4.9, 11.7 Hz, 1H), 1.77 (td, J=2.3, 12.9 Hz, 1H), 1.72-1.59 (m, 3H), 0.52 (s, 3H). HRMS (ESI) (m/z) calc'd for C₃₀H₃₃NaNO₃ [M+Na]³⁰: 478.2353, found 478.2347.

### Ketone 1

To a solution of isoquinoline 17 (19.0 mg, 41.7 μmol, 1.00 equiv) in acetone (1.4 mL) and water (350 μL) was added PTSA (20.9 mg, 83.4 μmol, 2.00 equiv) and the reaction mixture was warmed to 55° C. After 14.5 hours, the reaction was cooled to room temperature and saturated NaHCO₃ solution (2 mL) and ethyl acetate (2.5 mL) were sequentially added to the reaction. The layers were separated and the aqueous layer was extracted with ethyl acetate (2×2.5 mL). The organic layers were combined, washed with brine (2 mL), dried over Na₂SO₄ and concentrated under reduced pressure. The resulting residue was then purified by flash chromatography (silica gel, eluent: 3:2→1:2 hexanes:EtOAc) to afford ketone 1 (15.0 mg, 87%).

¹H NMR (500 MHz, CDCl₃)=9.23 (s, 1H), 8.48 (d, J=5.9 Hz, 1H), 7.80 (s, 1H), 7.78 (d, J=8.3 Hz, 1H), 7.65 (d, J=5.9 Hz, 1H), 7.61 (d, J=8.3 Hz, 1H), 5.91 (s, 1H), 5.40-5.35 (m, 1H), 3.15 (t, J=10.0 Hz, 1H), 2.94 (d, J=15.1 Hz, 1H), 2.68 (d, J=15.1 Hz, 1H), 2.67-2.59 (m, 1H), 2.58-2.41 (m, 4H), 2.41-2.24 (m, 3H), 2.24-2.10 (m, 2H), 2.04 (tt, J=4.6, 13.2 Hz, 1H), 1.96 (dd, J=5.4, 17.6 Hz, 1H), 1.86 (dq, J=5.1, 12.1 Hz, 1H), 1.80-1.67 (m, 2H), 0.55 (s, 3H). HRMS (ESI) (m/z) calc'd for C₂₈H₃₀NO₂ [M+H]⁺: 412.2271, found 412.2288.

### General Method for Reductive Amination

To a solution of ketone (1.00 equiv) in THF and i-PrOH (3:1, 0.02 M) was added amine (4.00 equiv) and Ti(Oi-Pr)₄ (2.50 equiv) sequentially. The reaction was then stirred at room temperature for 18 hours. The reaction mixture was cooled to −20° C. and NaBH₄ (1.50 equiv) was added. Once the reaction was done, saturated NaHCO₃ solution was added and the mixture was filtered through a pad of celite. The aqueous layer was extracted with CHCl₃ and the combined organic layers was washed with brine, dried over Na₂SO₄ and concentrated under reduced pressure.

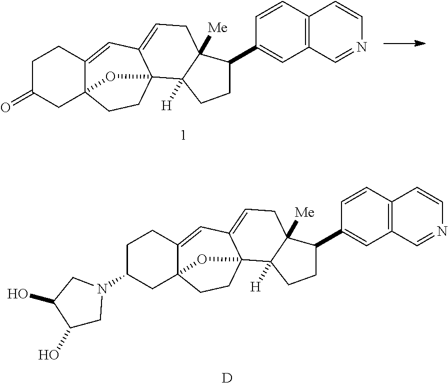

### C3 α-(3S,4S)-Pyrrolidine-3,4-Diol D

The crude mixture was purified sequentially by flash chromatography (silica gel, eluent: 10:1 EtOAc:2M NH₃ solution in MeOH→90:9:1 CHCl₃:MeOH:5N NH₄OH (aq)) to afford C3 α-(3S,4S)-pyrrolidine-3,4-diol D (25 mg, 60%).

¹H NMR (600 MHz, methanol-d₄) δ=9.19 (s, 1H), 8.37 (d, J=5.9 Hz, 1H), 7.96 (s, 1H), 7.87 (d, J=8.8 Hz, 1H), 7.79 (d, J=5.9 Hz, 1H), 7.73 (dd, J=1.5, 8.5 Hz, 1H), 5.71 (d, J=1.2 Hz, 1H), 5.26 (d, J=2.9 Hz, 1H), 4.04 (t, J=3.8 Hz, 2H), 3.30 (td, J=1.5, 3.4 Hz, 1H), 3.21 (t, J=10.6 Hz, 1H), 3.06 (dd, J=5.9, 10.0 Hz, 2H), 2.63 (dd, J=3.5, 10.6 Hz, 2H), 2.49 (dd, J=8.5, 11.4 Hz, 1H), 2.46-2.35 (m, 4H), 2.34-2.20 (m, 3H), 2.16 (td, J=4.6, 9.0 Hz, 1H), 2.09 (d, J=12.3 Hz, 1H), 2.05-1.90 (m, 4H), 1.88 (dd, J=5.3, 17.6 Hz, 1H), 1.76-1.66 (m, 2H), 1.61 (dt, J=7.6, 10.6 Hz, 1H), 1.29 (dq, J=5.3, 12.3 Hz, 1H), 0.54 (s, 3H). HRMS (ESI) (m/z) calc'd for C₃₂H₃₉N₂O₃ [M+H]⁺: 499.2955, found 499.2960.

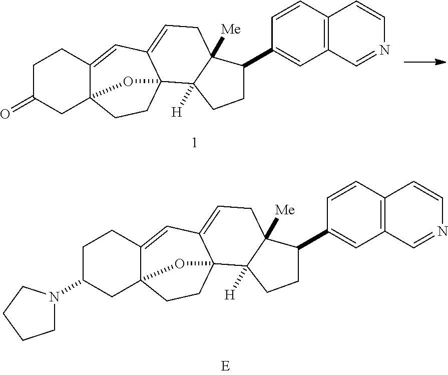

### C3 α-Pyrrolidine E

The crude mixture was purified by preparative TLC (silica gel, eluent: 20:10:3 EtOAc:Hexanes:2M NH₃ solution in MeOH) to afford α-pyrrolidine 19A (2.5 mg, 55%). ¹H NMR (500 MHz, CDCl₃) δ=9.22 (s, 1H), 8.48 (d, J=5.9 Hz, 1H), 7.79 (s, 1H), 7.75 (d, J=8.2 Hz, 1H), 7.62 (d, J=5.3 Hz, 1H), 7.59 (d, J=8.8 Hz, 1H), 5.72 (s, 1H), 5.27 (d, J=2.9 Hz, 1H), 3.14 (t, J=10.0 Hz, 1H), 2.63 (br. s., 4H), 2.52 (dd, J=8.8, 11.2 Hz, 1H), 2.42-2.29 (m, 3H), 2.28-2.15 (m, 5H), 2.12 (d, J=12.3 Hz, 1H), 2.10-2.00 (m, 2H), 1.93 (dd, J=5.3, 17.0 Hz, 1H), 1.90-1.83 (m, 2H), 1.80 (br. s., 4H), 1.72 (td, J=8.8, 12.9 Hz, 1H), 1.63 (br. s., 1H), 1.37 (dq, J=3.5, 11.7 Hz, 1H), 0.53 (s, 3H). HRMS (ESI) (m/z) calc'd for C₃₂H₃₉N₂O [M+H]⁺: 467.3057, found 467.3064.

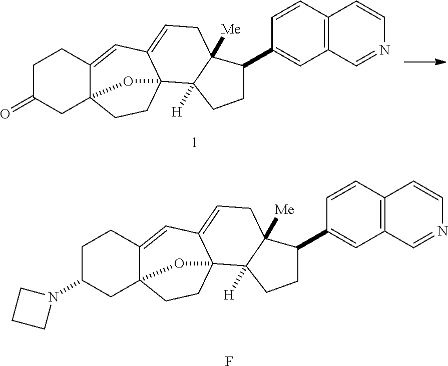

### C3 α-Azetidine F

The crude mixture was purified by preparative TLC (silica gel, eluent: 1:1 EtOAc:MeOH) to afford α-azetidine 18A (ca. 1.5 mg, 38%). ¹H NMR (500 MHz, CDCl₃) δ=9.24 (s, 1H), 8.50 (d, J=5.4 Hz, 1H), 7.80 (s, 1H), 7.77 (d, J=8.8 Hz, 1H), 7.64 (d, J=5.9 Hz, 1H), 7.60 (d, J=8.8 Hz, 1H), 5.74 (s, 1H), 5.28 (br. s., 1H), 3.24 (br. s., 4H), 3.16 (t, J=9.8 Hz, 1H), 2.54 (dd, J=8.8, 11.2 Hz, 1H), 2.42-2.30 (m, 3H), 2.30-2.13 (m, 5H), 2.12-2.00 (m, 2H), 1.95 (dd, J=5.4, 18.1 Hz, 1H), 1.93-1.78 (m, 3H), 1.74 (td, J=8.3, 12.2 Hz, 1H), 1.67-1.54 (m, 3H), 1.11 (q, J=12.2 Hz, 1H), 0.55 (s, 3H). HRMS (ESI) (m/z) calc'd for C₃₁H₃₇N₂O [M+H]⁺: 453.2906, found 453.2900.

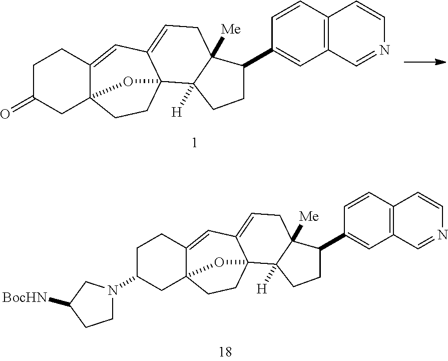

C3 α-(R)-3-(Boc-amino)pyrrolidine 18 The crude mixture was purified sequentially by flash chromatography (silica gel, eluent: 1:2 EtOAc:hexanes+5% 2M NH₃ solution in MeOH→1:1 EtOAc:hexanes+5% 2M NH₃ solution in MeOH) to afford C3 α-(R)-3-(Boc-amino)pyrrolidine 18 (105 mg, 65%). HRMS (ESI) (m/z) calc'd for C₃₇H₄₈N₃O [M+H]⁺: 582.3690, found 582.3680.

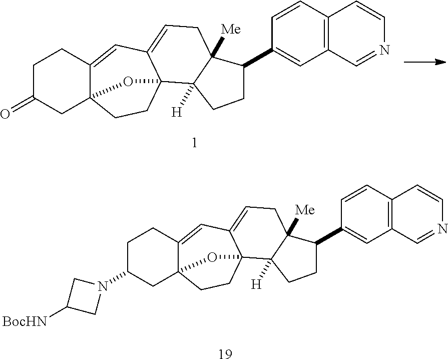

### C3 α-3-(Boc-Amino)Azetidine 19

The crude mixture was purified sequentially by flash chromatography (silica gel, eluent: 40:1 DCM:MeOH→20:1 DCM:MeOH) to afford C3 α-3-(Boc-amino)pyrrolidine 19 (70 mg, 40%). HRMS (ESI) (m/z) calc'd for C₃₆H₄₆N₃O₃ [M+H]⁺: 568.3534, found 568.3545.

### General Method for Boc-Deprotection

Boc-amine was stirred in DCM and TFA (6:1, 0.025 M) for 2 hours and the mixture was concentrated under reduced pressure.

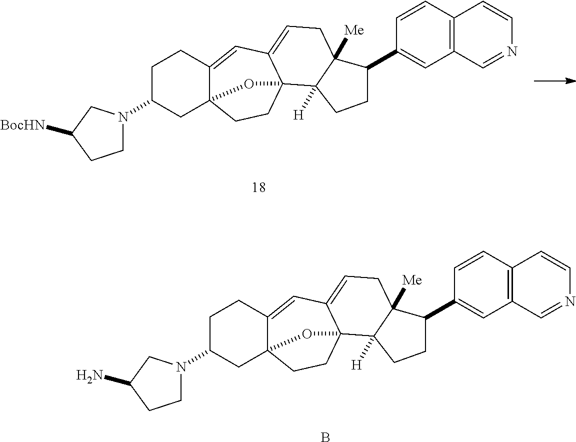

### C3 α-(R)-3-Aminopyrrolidine B

The crude mixture was purified sequentially by flash chromatography (silica gel, eluent: 90:9:1 CHCl₃:MeOH:5N NH₄OH (aq)) to afford C3 α-(R)-3-aminopyrrolidine B (85 mg, 99%).

¹H NMR (500 MHz, CDCl₃) δ=9.23 (s, 1H), 8.50 (d, J=5.9 Hz, 1H), 7.80 (s, 1H), 7.77 (d, J 8.3 Hz, 1H), 7.63 (d, J=5.4 Hz, 1H), 7.60 (dd, J=1.2, 8.5 Hz, 1H), 5.74 (d, J=1.5 Hz, 1H), 5.28 (d, J=2.9 Hz, 1H), 3.57-3.50 (m, 1H), 3.16 (t, J=10.0 Hz, 1H), 2.94 (dd, J=6.8, 9.3 Hz, 1H), 2.82 (dt, J=5.6, 8.7 Hz, 1H), 2.67 (dd, J=8.3, 15.1 Hz, 1H), 2.53 (dd, J=8.3, 11.2 Hz, 1H), 2.44-2.30 (m, 5H), 2.29-2.14 (m, 5H), 2.14-1.99 (m, 3H), 1.95 (dd, J=5.4, 17.1 Hz, 1H), 1.87 (dq, J=5.4, 12.2 Hz, 1H), 1.84 (t, J=12.2 Hz, 1H), 1.73 (td, J=8.5, 12.3 Hz, 1H), 1.63 (dt, J=7.8, 10.7 Hz, 1H), 1.52 (tdd, J=5.6, 7.6, 13.0 Hz, 1H), 1.36 (dq, J=4.9, 12.7 Hz, 1H), 0.55 (s, 3H); HRMS (ESI) (m/z) calc'd for C₃₂H₄₀N₃O [M+H]⁺: 482.3166, found 482.3152.

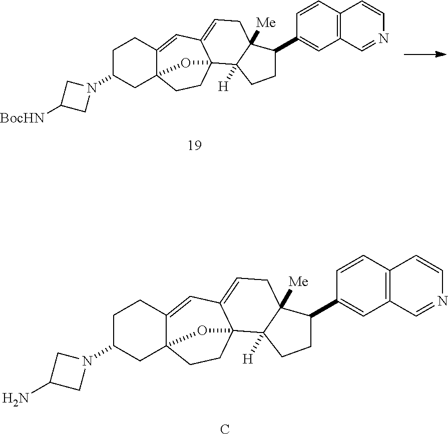

### C3 α-3-Aminoazetidine C

The crude mixture was purified sequentially by flash chromatography (silica gel, eluent: 90:9:1 CHCl₃:MeOH:5N NH₄OH (aq)) to afford C3 α-3-aminopyrrolidine C (50 mg, 87%).

¹H NMR (600 MHz, CDCl₃-d) 8=9.22 (s, 1H), 8.48 (d, J=5.3 Hz, 1H), 7.78 (s, 1H), 7.75 (d, J=8.8 Hz, 1H), 7.62 (d, J=5.9 Hz, 1H), 7.58 (d, J=8.8 Hz, 1H), 5.75 (s, 1H), 5.30 (s, 2H), 4.10-3.72 (m, 4H), 3.23-3.06 (m, 2H), 2.51 (dd, J=8.8, 11.2 Hz, 1H), 2.45-2.30 (m, 3H), 2.26 (t, J=11.2 Hz, 1H), 2.23-2.13 (m, 3H), 2.08-2.00 (m, 1H), 2.00-1.77 (m, 5H), 1.77-1.69 (m, 1H), 1.62 (d, J=7.6 Hz, 1H), 1.27 (br. s., 1H), 0.53 (s, 3H); HRMS (ESI) (m/z) calc'd for C₃₁H₃₈N₃O [M+H]⁺: 468.3009, found 468.3000.

### Example 3: Panlab Assay Screening

Compounds A, B, C, and D were tested in Panlab assays to evaluate potential off-target activity using standard assay kits. Methods employed in these studies have been adapted from the scientific literature to maximize reliability and reproducibility. Reference standards were run as an integral part of each assay to ensure the validity of the results obtained.

IC₅₀ values were determined by a non-linear, least squares regression analysis using MathIQ™ (ID Business Solutions Ltd., UK). Where inhibition constants (Ki) are presented, the Kᵢ values were calculated using the equation of Cheng and Prusoff (Cheng, Y., Prusoff, W. H., Biochem. Pharmacol. 22:3099-3108, 1973) using the observed IC₅₀ of the tested compound, the concentration of radioligand employed in the assay, and the historical values for the KD of the ligand (obtained experimentally at Eurofins Panlabs, Inc.). Where presented, the Hill coefficient (nH), defining the slope of the competitive binding curve, was calculated using MathIQ™. Hill coefficients significantly different than 1.0 may suggest that the binding displacement does not follow the laws of mass action with a single binding site.

### Compound A

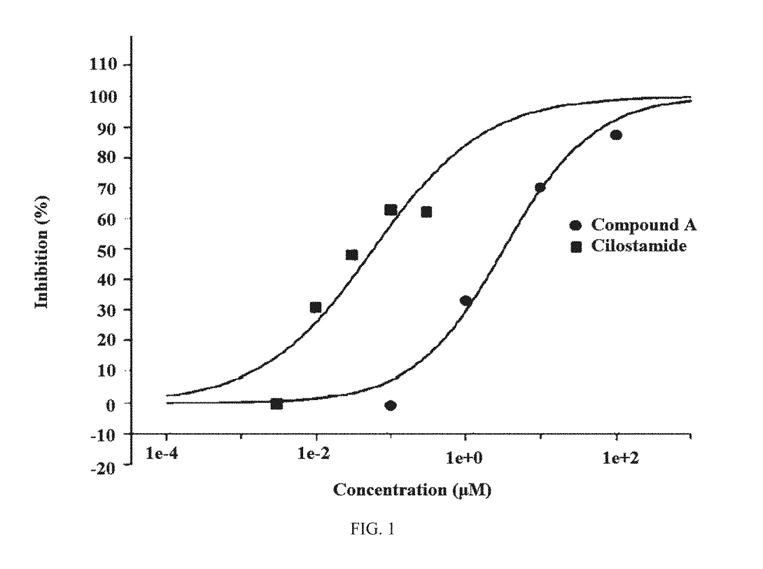

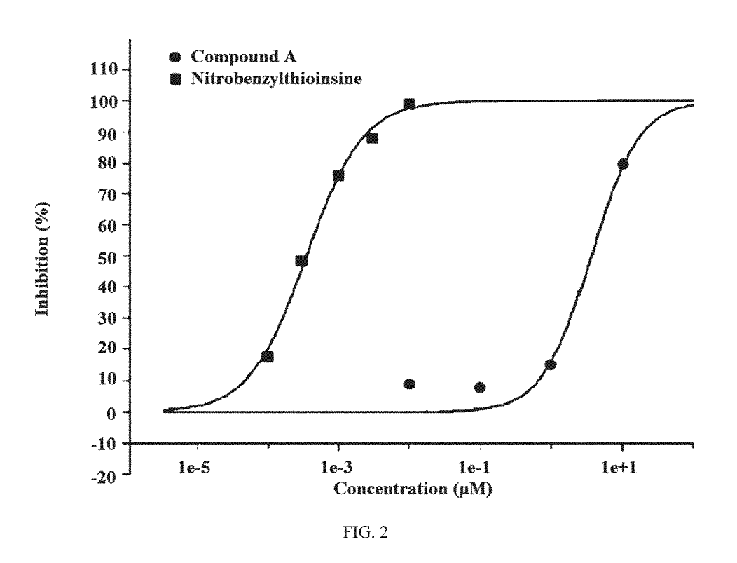

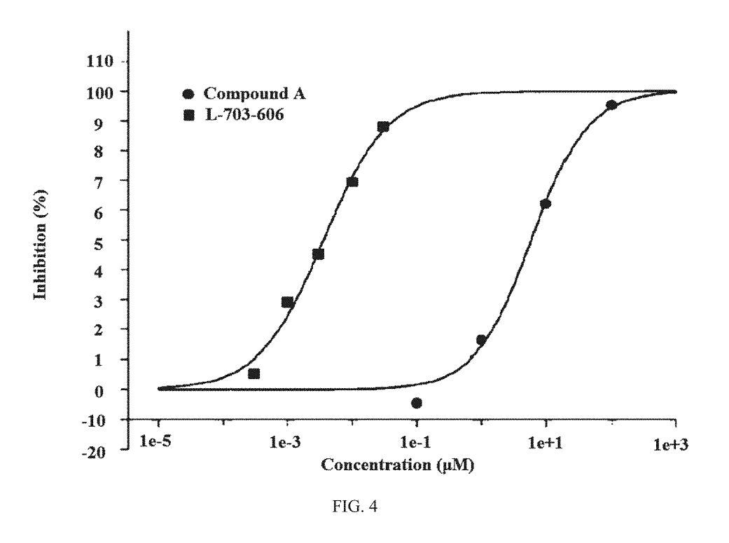

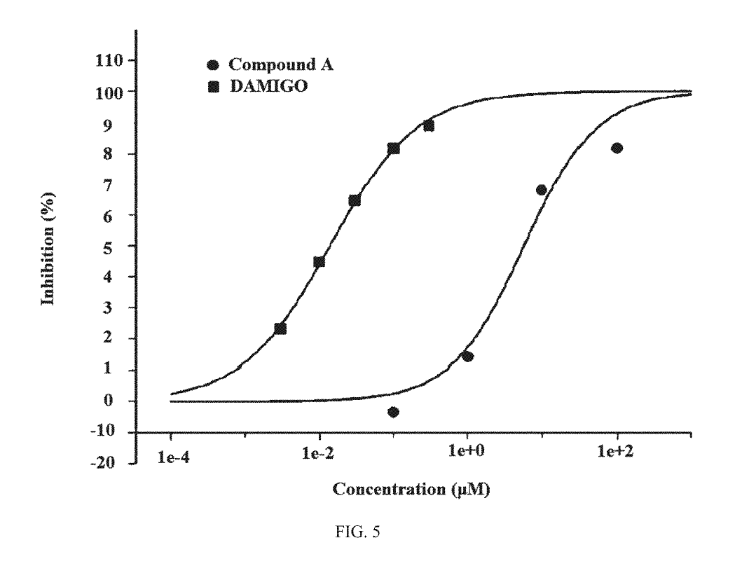

Compound A was tested in over 100 Panlab assays at 10 μM and only had greater than 50% inhibition against 8 targets. Follow up IC₅₀ testing showed that the most potent off-target activity was only 3.26 μM (FIG. 1), but even with this off-target activity, Compound A has a therapeutic index of nearly a 1000 and as such is quite selective. The dose response curve for Compound A against the off-target as well as a control compound is given in FIGS. 1, 2, 3, 4, and 5 corresponding to Phosphodiesterase PDE3, Transporter Adenosine, Transporter Dopamine, Tachykinin NK₁, and Opiate μ(OP3, MOP) respectively.

### Compound B

Compound B was tested in a subset of 30 Panlab assays that included the hits from screening Compound F in addition to the Panlab hERG and sodium channel assay. This allows direct comparison of Compound B to Compound F in terms of off-target profile. The results from the hERG and sodium channel assay (34% and 65% inhibition at 10 μM respectively) were consistent with those found in Example 4 showing Compound B to be a micromolar inhibitor of hERG. Compound B only showed greater than 50% inhibition at a 10 μM concentration against 8 targets.

### Compound C

Compound C was tested in a subset of 30 Panlab assays that included the hits from screening Compound F in addition to the Panlab hERG and sodium channel assay. The results from the hERG and sodium channel assay (49% and 66% inhibition at 10 μM respectively) were consistent with those found in Example 4 showing Compound C to be a micromolar inhibitor of hERG. Compound C only showed greater than 75% inhibition at 10 μM concentration against 4 targets.

### Compound D

Compound D was tested in a subset of 30 Panlab assays that included the hits from screening Compound F in addition to the Panlab hERG and sodium channel assay. The results from the hERG and sodium channel assay (20% and 65% inhibition at 10 μM respectively) were consistent with those found in Example 4 showing Compound D to be a micromolar inhibitor of hERG. Compound D only showed greater than 75% inhibition at a 10 μM concentration against 3 targets.

### Compound F

Compound F was tested in over 100 Panlab assays and was found to have greater than 50% inhibition of over 20 targets at 10 μM. Of the targets that Compound F inhibited, only 12 were with inhibition of greater than or equal to 75%.

### Example 4: Determination of hERG Activity

The in vitro effects of compounds A, B, C, D, E, and F on the hERG (human ether-à-go-go-related gene) potassium channel current (a surrogate for I_{Kr}, the rapidly activating, delayed rectifier cardiac potassium current) expressed in mammalian cells were evaluated at room temperature using the QPatch HT (Sophion Bioscience A/S, Denmark), an automatic parallel patch clamp system. The test articles were evaluated at 1 μM, 3 μM, 10 μl and 30 μM. Each test article concentration was tested in at least three cells. The duration of exposure to each test article concentration was a minimum of 3 minutes. A positive control (Cisapride. Table 1) tested within the expected range of % inhibitions.

TABLE 1
hERG Inhibition of Cisapride

| | Mean % | | | | Individual Data |
|---|---|---|---|---|---|
| Concentration | hERG | Standard | Standard | | Points |
| (μM) | Inhibition | Deviation | Error | N | (% Inhibition) |
| 0.05 | 72.7 | 9.5 | 3.9 | 6 | 77.9 |
| | | | | | 77.1 |
| | | | | | 74.2 |
| | | | | | 71.0 |
| | | | | | 54.8 |
| | | | | | 81.3 |

### Compound A

Compound A was found to have an IC₅₀ greater than 30 μM in the hERG assay (Table 2). This constitutes a major improvement over prior Cortistatin A analogs (see Compounds E and F) and further suggests that Compound A is an unprecedented selective inhibitor of CDK8 and CDK19 from the Cortistatin family. Discussion of the minor turbidity observed at 10 μM and 30 μM is presented in Example 5.

TABLE 2
hERG Inhibition of Compound A

| | | | | | | Individual |
|---|---|---|---|---|---|---|
| | Concen- | Mean % | | | | Data Points |
| IC₅₀ | tration | hERG | Standard | Standard | | (% |
| (μM) | (μM) | Inhibition | Deviation | Error | N | Inhibition) |
| >30 | 1 | 4.0 | 1.7 | 0.8 | 4 | 6.0 |
| | | | | | | 3.9 |
| | | | | | | 1.9 |
| | | | | | | 4.4 |
| | 3 | 9.2 | 3.6 | 1.8 | 4 | 10.0 |
| | | | | | | 13.4 |
| | | | | | | 4.6 |
| | | | | | | 9.0 |
| | 10* | 19.6 | 3.6 | 1.8 | 4 | 17.6 |
| | | | | | | 24.5 |
| | | | | | | 16.3 |
| | | | | | | 20.0 |
| | 30* | 43.7 | 10.3 | 5.1 | 4 | 35.5 |
| | | | | | | 42.9 |
| | | | | | | 38.0 |
| | | | | | | 58.4 |

*TurboSol analysis indicates that there may be a solubility issue at this concentration in this test article. Visible precipitation in the form of cloudiness was observed.

### Compound B

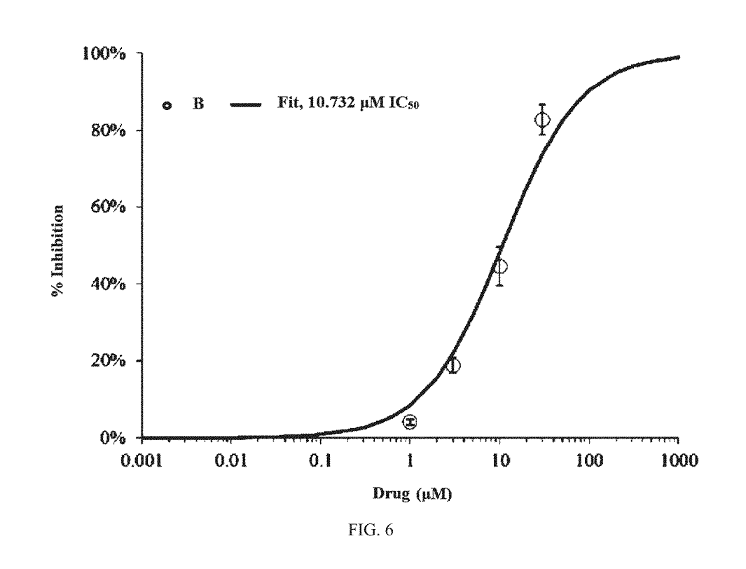

Compound B was found to have an IC₅₀ of approximately 10 μM in the hERG assay (Table 3). This constitutes a major improvement over prior Cortistatin A analogs (see Compounds E and F) and further suggests that Compound B is an unprecedentedly selective inhibitor of CDK8 and CDK19 from the Cortistatin family. The directly analogous unsubstituted pyrolidine (Compound E) has sub-micromolar hERG activity. The corresponding dose response curve is presented in FIG. 6. Discussion of the minor turbidity observed at 30 μM is presented in Example 5.

TABLE 3
hERG Inhibition of Compound B

| | | | | | | Individual |
|---|---|---|---|---|---|---|
| | Concen- | Mean % | | | | Data Points |
| IC₅₀ | tration | hERG | Standard | Standard | | (% |
| (μM) | (μM) | Inhibition | Deviation | Error | N | Inhibition) |
| 10.732 | 1 | 4.1 | 1.4 | 0.7 | 4 | 3.2 |
| | | | | | | 2.7 |
| | | | | | | 5.4 |
| | | | | | | 5.2 |
| | 3 | 18.9 | 3.9 | 2.0 | 4 | 17.1 |
| | | | | | | 14.4 |
| | | | | | | 21.2 |
| | | | | | | 23.0 |
| | 10 | 44.5 | 10.1 | 5.1 | 4 | 46.9 |
| | | | | | | 30.0 |
| | | | | | | 47.7 |
| | | | | | | 53.6 |
| | 30* | 82.7 | 7.9 | 4.0 | 4 | 89.4 |
| | | | | | | 73.0 |
| | | | | | | 79.6 |
| | | | | | | 89.0 |

*TurboSol analysis indicates that there may be a solubility issue at this concentration in this test article. Visible precipitation was observed.

### Compound C

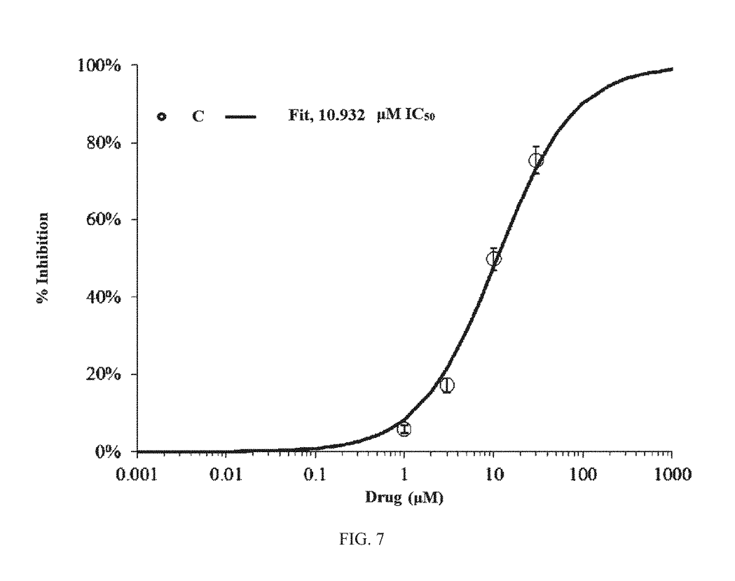

Compound C was found to have an IC₅₀ of approximately 10 μM in the hERG assay (Table 4). This constitutes a major improvement over prior Cortistatin A analogs (see Compounds E and F) and further suggests that Compound C is an unprecedentedly selective inhibitor of CDK8 and CDK19 from the Cortistatin family. The directly analogous unsubstituted azetidine (Compound F) has sub-micromolar hERG activity. The corresponding dose response curve is presented in FIG. 7. Discussion of the minor turbidity observed at 30 μM is presented in Example 5.

TABLE 4
hERG Inhibition of Compound C

| | | | | | | Individual |
|---|---|---|---|---|---|---|
| | Concen- | Mean % | | | | Data Points |
| IC₅₀ | tration | hERG | Standard | Standard | | (% |
| (μM) | (μM) | Inhibition | Deviation | Error | N | Inhibition) |
| 10.932 | 1 | 5.8 | 1.9 | 1.0 | 4 | 5.8 |
| | | | | | | 6.8 |
| | | | | | | 3.1 |
| | | | | | | 7.6 |
| | 3 | 17.1 | 3.7 | 1.8 | 4 | 12.7 |
| | | | | | | 15.6 |
| | | | | | | 19.5 |
| | | | | | | 20.7 |
| | 10 | 49.8 | 5.9 | 2.9 | 4 | 45.8 |
| | | | | | | 47.7 |
| | | | | | | 58.5 |
| | | | | | | 47.2 |
| | 30* | 75.5 | 6.9 | 3.5 | 4 | 66.9 |
| | | | | | | 78.3 |
| | | | | | | 83.2 |
| | | | | | | 73.8 |

*TurboSol analysis indicates that there may be a solubility issue at this concentration in this test article. Visible precipitation was observed.

### Compound D

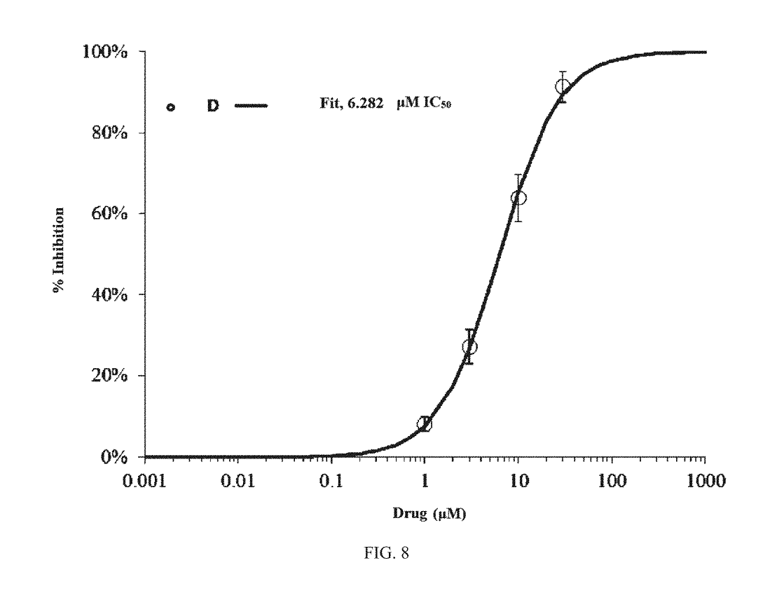

Compound D was found to have single digit micromolar activity in the hERG assay (Table 5). This constitutes a major improvement over prior Cortistatin A analogs (see Compounds E and F) and further suggests that Compound D is an unprecedentedly selective inhibitor of CDK8 and CDK19 from the Cortistatin family. The directly analogous unsubstituted pyrolidine (Compound E) has sub-micromolar hERG activity. The corresponding dose response curve is presented in FIG. 8. Discussion of the minor turbidity observed at 30 μM is presented in Example 5.

TABLE 5
hERG Inhibition of Compound D

| | | | | | | Individual |
|---|---|---|---|---|---|---|
| | Concen- | Mean % | | | | Data Points |
| IC₅₀ | tration | hERG | Standard | Standard | | (% |
| (μM) | (μM) | Inhibition | Deviation | Error | N | Inhibition) |
| 6.282 | 1 | 8.0 | 3.0 | 1.7 | 3 | 6.4 |
| | | | | | | 6.2 |
| | | | | | | 11.5 |
| | 3 | 27.3 | 7.3 | 4.2 | 3 | 25.1 |
| | | | | | | 21.3 |
| | | | | | | 35.3 |
| | 10 | 63.9 | 10.2 | 5.9 | 3 | 71.6 |
| | | | | | | 52.4 |
| | | | | | | 67.7 |
| | 30* | 91.3 | 6.6 | 3.8 | 3 | 97.9 |
| | | | | | | 84.6 |
| | | | | | | 91.4 |

*TurboSol analysis indicates that there may be a solubility issue at this concentration in this test article. Visible precipitation was observed.

### Compound E

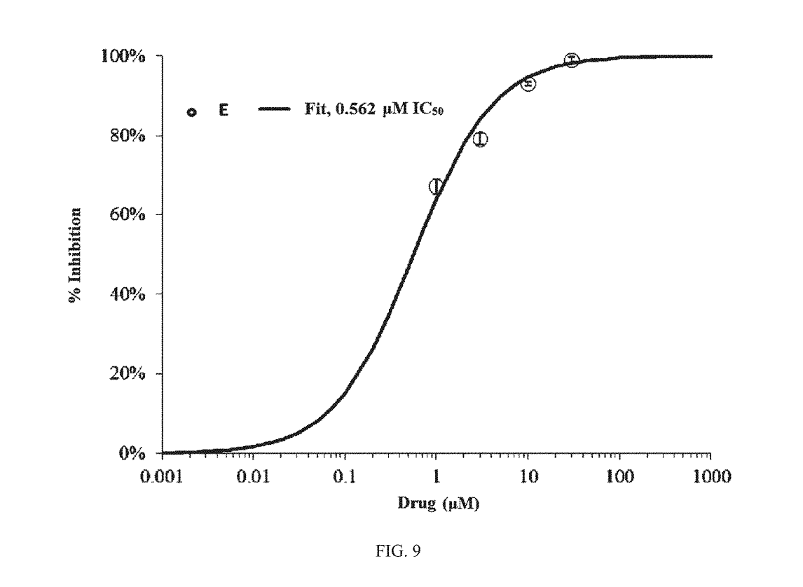

Compound E was found to have sub-micromolar activity in the hERG assay (Table 6). The directly analogous substituted pyrrolidines (Compounds B and D) have 10 to 100 fold less hERG activity. The corresponding dose response curve is presented in FIG. 9. Discussion of the minor turbidity observed at 30 μM is presented in Example 5.

TABLE 6
hERG Inhibition of Compound E

| | | | | | | Individual |
|---|---|---|---|---|---|---|
| | Concen- | Mean % | | | | Data Points |
| IC₅₀ | tration | hERG | Standard | Standard | | (% |
| (μM) | (μM) | Inhibition | Deviation | Error | N | Inhibition) |
| 0.562 | 1 | 67.2 | 3.8 | 1.9 | 4 | 72.4 |
| | | | | | | 67.5 |
| | | | | | | 65.6 |
| | | | | | | 63.4 |
| | 3 | 79.2 | 2.9 | 1.4 | 4 | 83.4 |
| | | | | | | 77.1 |
| | | | | | | 77.3 |
| | | | | | | 79.0 |
| | 10 | 93.0 | 1.0 | 0.5 | 4 | 93.8 |
| | | | | | | 92.6 |
| | | | | | | 91.8 |
| | | | | | | 93.6 |
| | 30* | 98.8 | 1.6 | 0.8 | 4 | 97.7 |
| | | | | | | 98.0 |
| | | | | | | 98.3 |
| | | | | | | 101.2 |

*TurboSol analysis indicates that there may be a solubility issue at this concentration in this test article. Visible precipitation was observed.

### Compound F

The objective of this study was to evaluate the in vitro effects of Compound F on three cardiac ion channel currents: I_{Kr}, the rapidly activating, delayed rectifier potassium current through hERG channel encoded by human ether-à-go-go-related gene; I_{Ks}, the slowly activating, delayed rectifier cardiac potassium channel current encoded by hKCNQ1/hKCNE1; and INa, the human cardiac Na+ channel current encoded by hNav1.5 in CHO or HEK293 cells (Table 7). This functional assay was conducted using higher throughput planar voltage-clamp technology PatchXpress 7000A.

Signal amplitudes were quantified at steady state during control (vehicle) and in the presence of increasing concentrations of the test compound. Concentration-dependent changes of signal in response to compound were expressed as percent block relative to control (pre-drug/trigger) and are reported as the mean±SEM (standard error) of the indicated number (N) of tested cells/wells. IC₅₀ values were determined where applicable by fitting the averaged concentration-response data (mean±SEM) with a Hill equation.

The hERG IC₅₀ was determined to be 0.37 μM in this functional voltage clamp assay conducted with the PatchXpress. At the highest tested concentration of 30 μM, the compound inhibited I_{Ks} by 68±15%. The calculated IC₅₀ value for inhibition of I_{Ks} by the compound in this functional assay was 17 μM. At the highest tested concentration of 30 μM, the compound inhibited INa by 98±1% at the pulsing rate of 3 Hz, and by 86±2% at the slower pulsing rate of 0.2 Hz, thus displaying significant rate-dependent inhibitory effects. The calculated IC₅₀ values at pulsing rates of 0.2 Hz and 3 Hz in this functional INa assay were 14 μM and 6.5 μM, respectively.

TABLE 7
Channel Blocking Data of Compound F
% Block Mean ± SEM (n)

| Concentration | | | |
|---|---|---|---|
| (μM) | hERG | I_{KS} | I_{Na} at 3 Hz |
| 0.12 | 21 ± 2 (3) | ND | ND |
| 0.37 | 51 ± 1 (3) | ND | ND |
| 1.1 | 77 ± 0 (3) | ND | ND |
| 3.3 | 94 ± 0 (4) | ND | 30 ± 5 (4) |
| 10 | 97 ± 0 (4) | 34 ± 7 (5) | 60 ± 3 (4) |
| 30 | 102 ± 1 (4) | 68 ± 15 (4) | 98 ± 1 (4) |
| IC₅₀ (μM) | 0.37 | 17 | 6.5 |
| IC₂₀ (μM) | 0.11 | ND | ND |

### Example 5: TurboSol

TurboSol evaluation was performed on the test articles to determine solubility in physiological saline solution (HB-PS+0.3% DMSO. The solubility limit for this experiment as determined by vehicle controls was 8.0×103 LSU (Light Scattering Units, horizontal black line). Based on the data obtained, there may be solubility issues in physiological saline solution (HB-PS+0.3% DMSO). Precipitation was visible.

### Compounds A and E

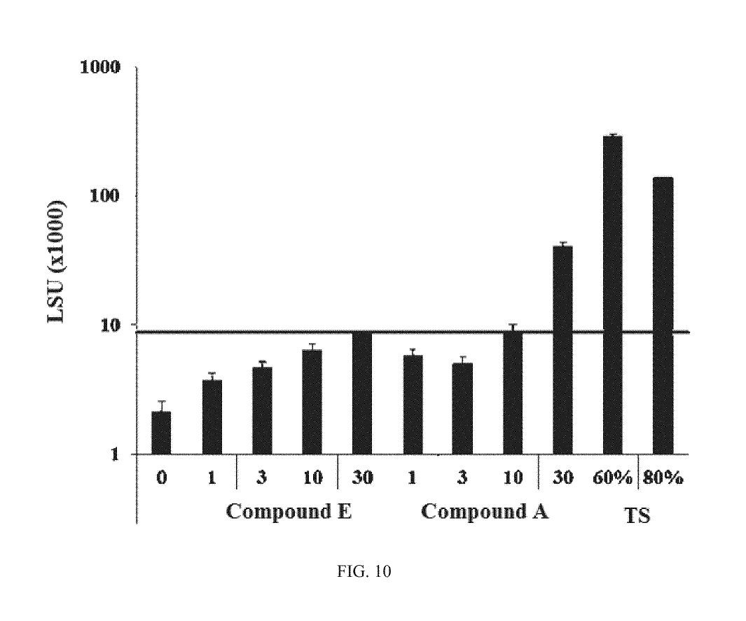

Compounds A and E were tested in the TurboSol assay to determine their solubility (Table 8 and FIG. 10). Compound A was found to be slightly insoluble at concentrations of 10 μM and 30 μM, but was far under the LSU readout for the 80% transmission standard. Compound E was found to be slightly insoluble at 30 μM.

TABLE 8
TurboSol Analysis of Compound A and Compound E

| | | | | | | Stan- | |
|---|---|---|---|---|---|---|---|
| | | FIG. | Concen- | | Average | dard | |
| | Com- | Leg- | tration | | LSU | Devi- | Thresh- |
| Plate | pound | end | (μM) | N | (×1000) | ation | old |
| 151007.IBU | Vehicle | A | 0 | 11 | 2.2 | 0.4 | 8.8 |
| | Compound E | | 1 | 3 | 3.7 | 0.5 | 8.8 |
| | | | 3 | 3 | 4.7 | 0.5 | 8.8 |
| | | | 10 | 3 | 6.4 | 0.6 | 8.8 |
| | | | 30* | 3 | 8.7 | 0.2 | 8.8 |
| | Compound A | | 1 | 3 | 5.8 | 0.6 | 8.8 |
| | | | 3 | 3 | 5.1 | 0.6 | 8.8 |
| | | | 10* | 3 | 9.0 | 1.0 | 8.8 |
| | | | 30* | 3 | 41.0 | 2.3 | 8.8 |
| | Transmit- | TS | 60% | 3 | 294.2 | 4.2 | 8.8 |
| | tance | | 80% | 3 | 138.8 | 1.8 | 8.8 |
| | Standard | | | | | | |

*TurboSol analysis indicates that there may be a solubility issue at this concentration in this test article. Visible precipitation was observed.
LSU: Light Scattering Unit
60% TS and 80% TS: Standard for 60% and 80% Transmittance, respectively.

### Compounds B and C

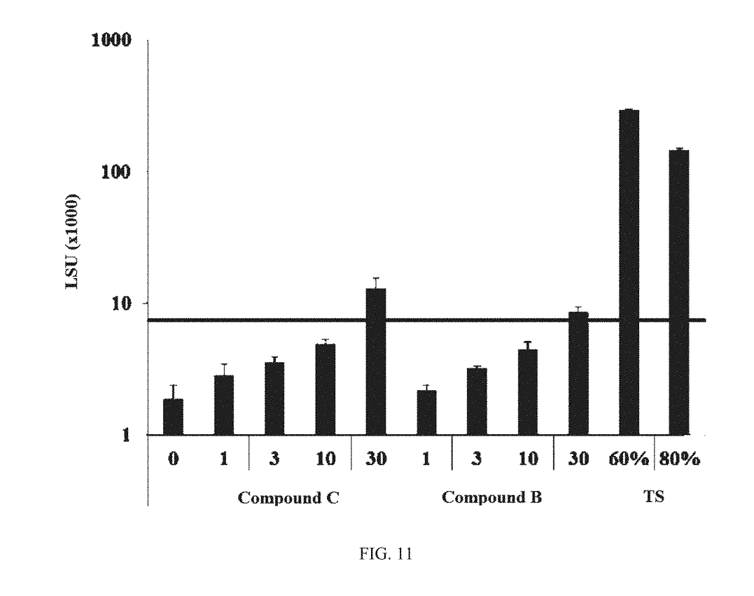

Compounds B and C were tested in the TurboSol assay to determine their solubility (Table 9 and FIG. 11). Compounds B and C were both found to be slightly insoluble at 30 μM concentrations, but were far under the LSU readout for the 80% transmission standard.

TABLE 9
TurboSol Analysis of Compound B and Compound C

| | | | | | | Stan- | |
|---|---|---|---|---|---|---|---|
| | | FIG. | Concen- | | Average | dard | |
| | Com- | Leg- | tration | | LSU | Devi- | Thresh- |
| Plate | pound | end | (μM) | N | (×1000) | ation | old |
| 151027.IBU | Vehicle | A | 0 | 12 | 1.9 | 0.5 | 7.4 |
| | Compound C | | 1 | 3 | 2.8 | 0.6 | 7.4 |
| | | | 3 | 3 | 3.5 | 0.3 | 7.4 |
| | | | 10 | 3 | 4.9 | 0.4 | 7.4 |
| | | | 30* | 3 | 13.0 | 2.2 | 7.4 |
| | Compound B | | 1 | 3 | 2.2 | 0.2 | 7.4 |
| | | | 3 | 3 | 3.2 | 0.1 | 7.4 |
| | | | 10 | 3 | 4.5 | 0.6 | 7.4 |
| | | | 30* | 3 | 8.6 | 0.8 | 7.4 |
| | Transmit- | TS | 60% | 3 | 294.2 | 7.4 | 7.4 |
| | tance | | 80% | 3 | 146.1 | 7.4 | 7.4 |
| | Standard | | | | | | |

*TurboSol analysis indicates that there may be a solubility issue at this concentration in this test article. Visible precipitation was observed.
LSU: Light Scattering Unit
60% TS and 80% TS: Standard for 60% and 80% Transmittance, respectively.

### Compound D

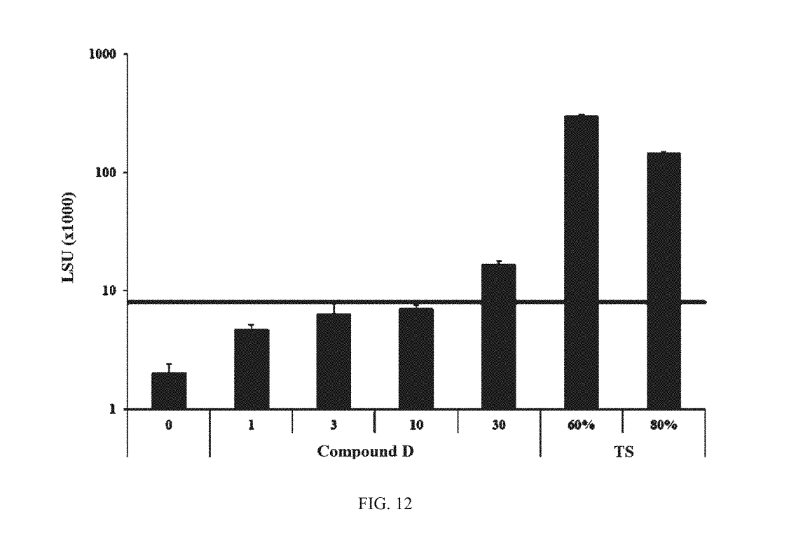

Compound D was tested in the TurboSol assay to determine its solubility (Table 10 and FIG. 12). Compound D was found to be slightly insoluble at 30 μM concentrations, but was far under the LSU readout for the 80% transmission standard.

TABLE 10
TurboSol Analysis of Compound D

| | | | | | | Stan- | |
|---|---|---|---|---|---|---|---|
| | | FIG. | Concen- | | Average | dard | |
| | Com- | Leg- | tration | | LSU | Devi- | Thresh- |
| Plate | pound | end | (μM) | N | (×1000) | ation | old |
| 150925.IBU | Vehicle | A | 0 | 12 | 2.0 | 0.4 | 8.0 |
| | Compound D | | 1 | 3 | 4.7 | 0.5 | 8.0 |
| | | | 3 | 3 | 6.4 | 1.5 | 8.0 |
| | | | 10 | 3 | 7.0 | 0.5 | 8.0 |
| | | | 30* | 3 | 16.6 | 1.3 | 8.0 |
| | Transmit- | TS | 60% | 3 | 300.5 | 8.0 | 8.0 |
| | tance | | 80% | 3 | 144.5 | 8.0 | 8.0 |
| | Standard | | | | | | |

*TurboSol analysis indicates that there may be a solubility issue at this concentration in this test article. Visible precipitation was observed.
LSU: Light Scattering Unit
60% TS and 80% TS: Standard for 60% and 80% Transmittance, respectively.

### Example 6: Tolerability Studies of Compound A and Compound D

Twenty-five female NSG mice were randomly assigned to various treatment groups as described in Table 11. All mice were treated for 7 days unless as indicated below.

On study days zero to six (seven doses), Compound A was administered via oral gavage in a volume of 10 mL/kg and Compound D was administered via intravenous injection into the lateral tail vein in a volume of 10 mL/kg. Body weights were recorded daily for fifteen days (days zero to fourteen) and animal health was assessed daily.

At doses of 10 mg/kg, 1 of 2 mice dosed with either Compound A or Compound D demonstrated >15% body weight loss by day 5 of dosing resulting in treatment holiday. Mice recovered during the additional 7 days of observational period. Both agents were tolerated (<6% body weight loss) at doses at or below 3 mg/kg.

Mice were monitored clinically for signs of morbidity and for body weights daily. Mice showing signs of morbidity were euthanized as per study guidelines. Tolerability is defined as the dose that did not show clinical signs of morbidity such as ruffled fur, hunch backing, labored breathing, recumbency or difficulty with ambulation, and body weight loss of more than 15%.

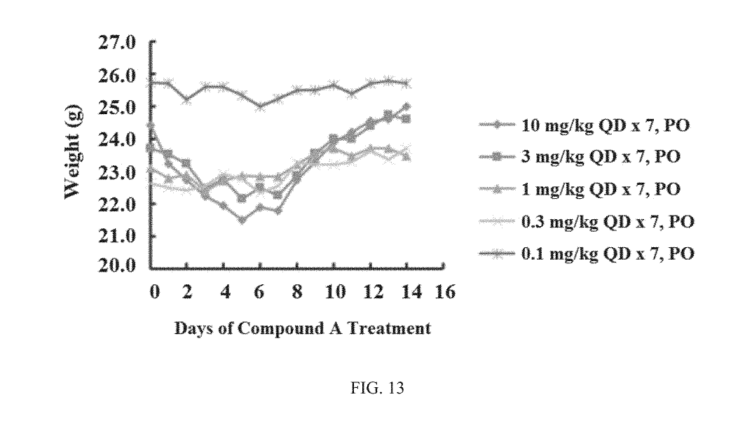

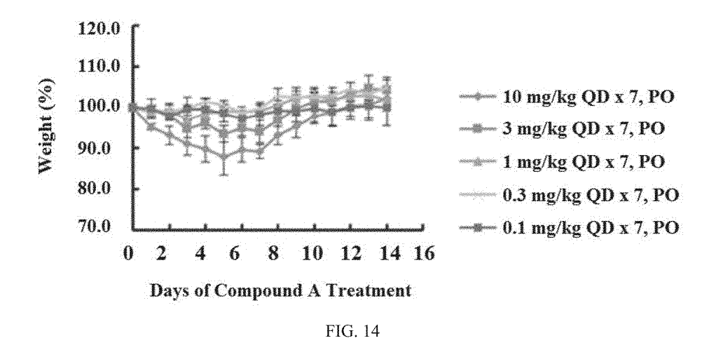

Compound A was well tolerated at or below 3 mg/kg with the mice returning to approximately or slightly higher than their initial weight at the end of the dosing period (FIG. 13). Mice in the 10 mg/kg dosing group exhibited a small (approximately 15%) initial loss in weight, but recovered to healthy weights after a dosing holiday (FIG. 14).

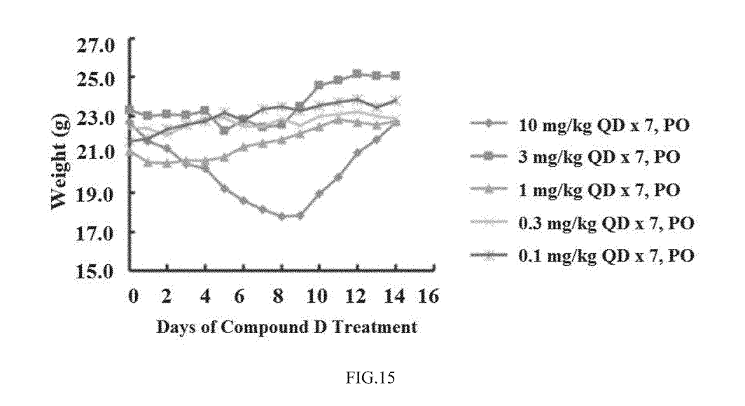

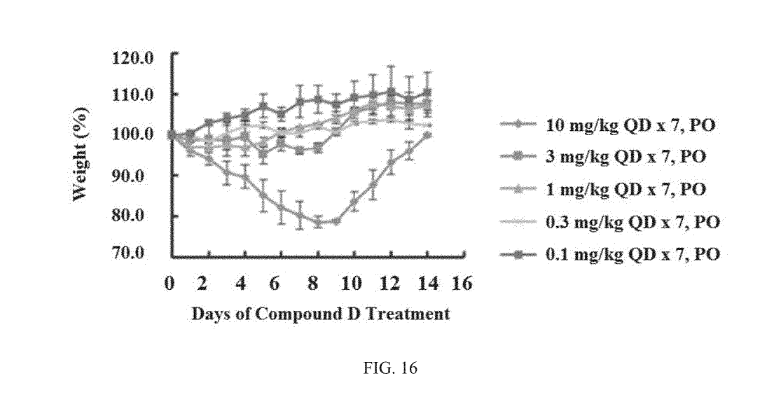

Compound D was well tolerated at doses at or below 3 mg/kg with the mice returning to approximately or slightly higher than their initial weight (FIG. 15). Mice in the 10 mg/kg dosing group exhibited an approximately 20% initial loss in weight, but recovered to healthy weights after a dosing holiday (FIG. 16).

TABLE 11
Study Groups of Tolerability Studies for Compound A and Compound D

| | | | | Dosing | Dosing | Number of |
|---|---|---|---|---|---|---|
| Group # | Test Agent | Dose | | Volume | Schedule | Animals |
| 1 | Compound A | 0.1 | mg/kg | 10 mL/kg | QD x 7, PO | 2 |
| 2 | Compound A | 0.3 | mg/kg | 10 mL/kg | QD x 7, PO | 3 |
| 3 | Compound A | 1 | mg/kg | 10 mL/kg | QD x 7, PO | 3 |
| 4 | Compound A | 3 | mg/kg | 10 mL/kg | QD x 7, PO | 3 |
| 5 | Compound A | 10 | mg/kg | 10 mL/kg | QD x 7, PO | 2 |
| 6 | Compound D | 10 | mg/kg | 10 mL/kg | QD x 7, IV | 2 |
| 7 | Compound D | 3 | mg/kg | 10 mL/kg | QD x 7, IV | 2 |
| 8 | Compound D | 1 | mg/kg | 10 mL/kg | QD x 7, IV | 3 |
| 9 | Compound D | 0.3 | mg/kg | 10 mL/kg | QD x 7, IV | 3 |
| 10 | Compound D | 0.1 | mg/kg | 10 mL/kg | QD x 7, IV | 2 |

### Example 7: Tolerability Studies of Cortistatin A and Compound F

### Compound F

Twenty seven mice were used for the tolerability study of Compound F. Mice were randomly assigned to various treatment groups with n=3 mice/group. In the low dose group of 0.3 mg/kg Compound F administered IP, mice were re-challenged after 8 days of drug holiday with a higher dose of 20 mg/kg Compound F, IP. All mice were treated for 7 days unless as indicated below.

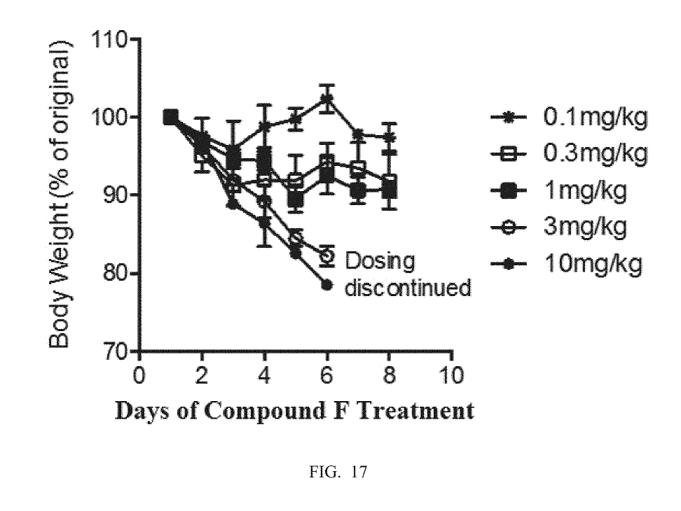

In the oral dosing group, Compound F at 3 mg/kg resulted in >15% body weight loss requiring drug holiday in 3 of 3 mice, while a dose of 10 mg/kg was not tolerated with >20% body weight loss. At the 1 mg/kg dose, the average body weight loss was ˜10% (FIG. 17)

In the IP dosing group, 20 mg/kg dose resulted in >15% body weight loss in 2 of 3 mice.

Mice were monitored clinically for signs of morbidity and for body weights daily. Mice showing signs of morbidity were euthanized as per study guidelines. Tolerability is defined as the dose that did not show clinical signs of morbidity such as ruffled fur, hunchbacking, labored breathing, recumbency or difficulty with ambulation, and body weight loss of more than 15%.

### Cortistatin A

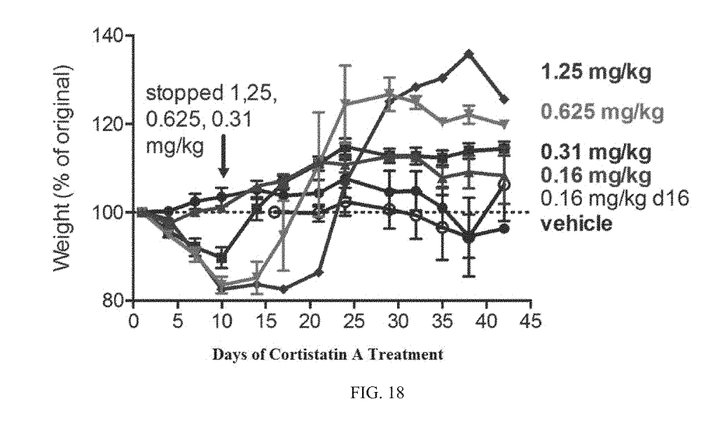

Mice were injected intravenously with MV4; 11-mCLP cells and imaged on days 3 and 7 post-injection. At this time mice were divided into seven treatment groups with mean bioluminescence˜8×10⁶ ph/s/cm²/sr (n=3 per group): cA @ 5 mg/kg, 2.5 mg/kg, 1.25 mg/kg, 0.625 mg/kg, 0.3125 mg/kg and 0.15625 mg/kg of Cortistatin A or vehicle. All treatments were administered IP in a volume of 10 mL/kg once daily. The drug showed varying levels of toxicity based on dose, with each group being dosed until body weight loss reached ˜15% (FIG. 18). Bioluminescence and weight data were collected every 3-4 days for seven weeks. Mice were sacrificed when they reached 20% weight loss or when moribund. Upon sacrifice, mice were fixed in bouin's fixative. Additionally, blood was drawn from the six mice sacrificed on day 64 and CBC analysis was done on a Hemavet 950FS.

### Example 8: Compound a, B, C, and D Kinome Profiling

A radiometric protein kinase assay (33PanQinase Activity Assay) was used for measuring the kinase activity of the 320 protein kinases. All kinase assays were performed in 96-well FlashPlates™ from Perkin Elmer (Boston, Mass., USA) in a 50 μl reaction volume. The reaction cocktail was pipetted in 4 steps in the following order:
1. 10 μl of non-radioactive ATP solution (in H2O)
2. 25 μl of assay buffer/[γ-33P]-ATP mixture
3. 5 μl of test sample in 10% DMSO
4. 10 μl of enzyme/substrate mixture

The assay for all protein kinases contained 70 mM HEPES-NaOH pH 7.5, 3 mM MgCl₂, 3 mM MnCl₂, 3 μM Na-orthovanadate, 1.2 mM DTT, ATP (variable amounts, corresponding to the apparent ATP-Km of the respective kinase), [γ-33P]-ATP (approx. 8×1005 cpm per well), protein kinase (variable amounts), and substrate (variable amounts). All PKC assays (except the PKC-mu and the PKC-nu assay) additionally contained 1 mM CaCl₂, 4 mM EDTA, 5 μg/ml phosphatidylserine and 1 μg/ml 1,2-dioleyl-glycerol.

The CAMKID, CAMK2A, CAMK2B, CAMK2D, CAMK4, CAMKK1, CAMKK2, DAPK2, EEF2K, MYLK, MYLK2 and MYLK3 assays additionally contained 1 μg/ml Calmodulin and 0.5 mM CaCl₂). The PRKG1 and PRKG2 assays additionally contained 1 μM cGMP. The DNA-PK assay additionally contained 2.5 μg/ml DNA. The protein kinase reaction cocktails were incubated at 30° C. for 60 minutes. The reaction was stopped with 50 μl of 2% (v/v) H₃PO₄, plates were aspirated and washed two times with 200 μl 0.9% (w/v) NaCl. Incorporation of 33Pi (counting of “cpm”) was determined with a microplate scintillation counter (Microbeta, Wallac). All protein kinase assays were performed with a BeckmanCoulter Biomek 2000/SL robotic system.

All protein kinases provided by ProQinase were expressed in Sf9 insect cells or in E. coli as recombinant GST-fusion proteins or His-tagged proteins, either as full-length or enzymatically active fragments. All kinases were produced from human cDNAs and purified by either GSHaffinity chromatography or immobilized metal. Affinity tags were removed from a number of kinases during purification. The purity of the protein kinases was examined by SDSPAGE/Coomassie staining, the identity was checked by mass spectroscopy.

Kinases from external vendors CAR=Carna Biosciences Inc.; INV=Life Technologies (Invitrogen Corporation); MIL=Merck-Millipore (Millipore Corporation) were expressed, purified and quality-controlled by virtue of the vendors readings.

For each kinase, the median value of the cpm of three wells was defined as “low control” (n=3). This value reflects unspecific binding of radioactivity to the plate in the absence of a protein kinase but in the presence of the substrate. Additionally, for each kinase the median value of the cpm of three other wells was taken as the “high control”, i.e. full activity in the absence of any inhibitor (n=3). The difference between high and low control of each enzyme was taken as 100% activity. As part of the data evaluation the low control of each kinase was subtracted from the high control value as well as from their corresponding “compounds values”. The residual activity (in %) for each compounds well was calculated by using the following formula:

Res. Activity (%)=100×[(signal of compounds−low control)/(high control−low control)]

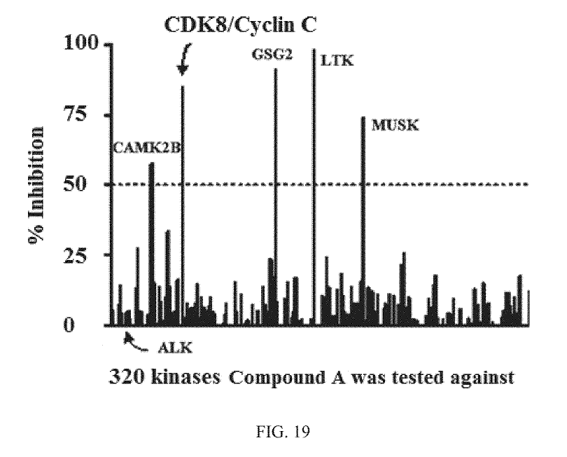

1 μM Compound A (100-times IC₅₀ for CDK8/Cyclin C inhibition) is selective for CDK8/Cyclin C among 320 kinases (FIG. 19). Testing was done using an in vitro kinase phosphorylation assay panel by the company ProQinase. Shown is the average percent inhibition with 2 replicates measured for each kinase. There were 5 kinases with an average of >50% inhibition by Compound A: CDK8/Cyclin C, CAMK2B, LTK and MUSK. However, only CDK8/Cyclin C is likely to be inhibited in cells or in vivo by Compound A for the following reasons: (1) GSG2 was ruled-out as an in cell target of Cortistatin A (see Shair Nature 2015), (2) replicates were inconsistent for CAMK2B (36%, 79%/o) and MUSK (36%, 111%) and (3) LTK inhibition is inconsistent with ALK inhibition. Compound A does not inhibit ALK in vitro (<10% inhibition) but LTK and ALK kinase domains are 790/o identical and FDA approved ALK inhibitor crizotinib (XALKORI) also inhibits LTK.

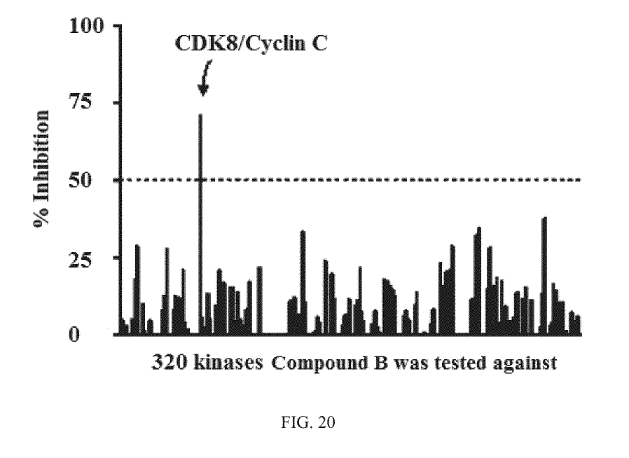

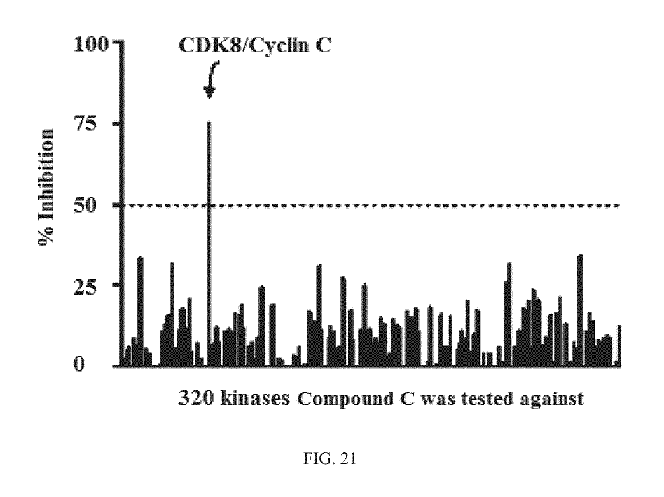

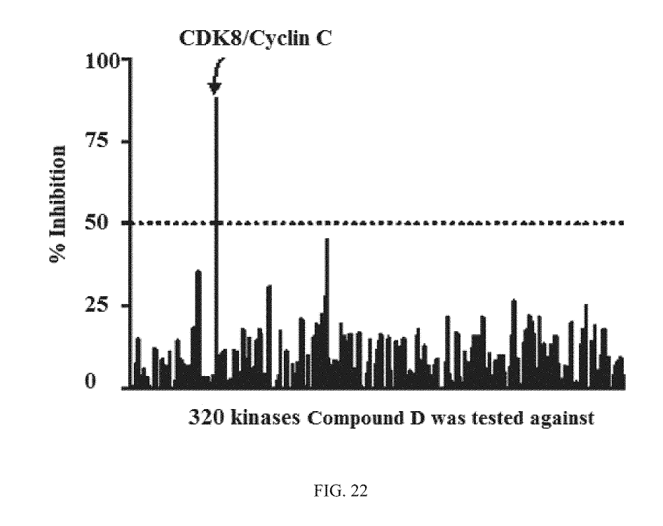

Upon retesting, CAMK2B, LTK and MUSK were not inhibited by up to 10 μM Compound A. It was previously determined that Cortistatin A, which also inhibited GSG2 in vitro, did not inhibit GSG2 in cellular lysate (Shair Nature, 2015). Therefore, CDK8/Cyclin C was the only validated kinase to be strongly inhibited by 1 μM Compound A in the kinome panel. 1 μM Compound B, C, and D were also selective for CDK8/Cyclin C in the kinome profiling as seen in FIG. 20, FIG. 21, and FIG. 22 respectively.

### Example 9: Compound a, B, C, and D CDK8 Binding IC₅₀

CDK8 inhibition in cells as measured by inhibition of Interferon-gamma (IFNγ)-stimulated STAT1-S727 phosphorylation. The compounds were provided as 200 μl of 1×10⁻⁰³ M/100% stock solutions in vials. The vials arrived in good condition and 2×100 μl each of the stock solutions were transferred into wells A2 to H2 of a 96 well “master plate” (barcoded “11273-UNH-01”), according to Table 12.

TABLE 12
Well ID of Compounds for CDK8 Binding Assay

| Number | Compound ID | Well ID |
|---|---|---|
| 1 | Compound A | A2 |
| 2 | Compound A | B2 |
| 3 | Compound D | A3 |
| 4 | Compound D | B3 |
| 5 | Compound C | A4 |
| 6 | Compound C | B4 |
| 7 | Compound B | A5 |
| 8 | Compound B | B5 |

Prior to testing, the 1×10⁻⁰³ M stock solutions in column 2 of the master plate were subjected to a serial, semi-logarithmic dilution using 100% DMSO as a solvent. This resulted in 10 distinct concentrations, with a dilution endpoint of 3×10⁻⁰⁸ M/100% DMSO in column 12. Column 1 and 7 were filled with 100% DMSO as controls. Subsequently, 1×10 μl from each well of the serial diluted copy plate were aliquoted with a 96 channel pipettor into a “compound dilution plate”, barcoded “11273-UNH-01”.

In the process, 90 μl H₂O was added to each well of the compound dilution plate. To minimize potential precipitation, water was added to the plate only a few minutes before the transfer of the compound solutions into the assay plates. The plate was shaken thoroughly, resulting in a “compound dilution plate/10% DMSO”. The compound dilution plate was discarded at the end of the working day.

For the assays, 5 μl solution from each well of the compound dilution plates/10% DMSO was transferred into the assay plates. The final volume of the assay was 50 μl. The compound was tested at 10 final assay concentrations in the range of 1×10₋₀₅ M to 3×10⁻¹⁰ M in duplicate. The final DMSO concentration in the reaction cocktails was 1% in all cases. Compound A was found to have IC₅₀s of 10.6 nM and 9.3 nM against CDK8. Compound B was found to have IC₅₀s of 5.5 nM and 12.7 nM against CDK8. Compound C was found to have IC₅₀s of 8.9 nM and 10.2 nM against CDK8. Compound D was found to have IC₅₀s of 6.4 nM and 6.5 nM against CDK8. Compounds A, B, C, and D were found to have average IC₅₀s of 9.9 nM, 9.1 nM, 9.5 nM, and 6.4 nM respectively (Table 13). Thus, Compounds A, B, C, and D are all very potent and selective inhibitors of CDK8. Compound A was additionally found to have a longer residence time than Cortistatin A.

TABLE 13
IC₅₀ and K_{d} values of Compounds A, B, C, and D compared to Cortistatin A

| | CA | Compound A | Compound B | Compound C | Compound D |
|---|---|---|---|---|---|
| IC₅₀ (nM) | 3.5 | 9.9 | 9.1 | 9.5 | 6.4 |
| K_{d} (nM) | 0.195 ± 0.02 | 0.23 ± 0.02 | 0.19 ± 0.05 | 0.22 ± 0.03 | 0.2 ± 0.02 |
| Residence time | 262 ± 34 | 447 | | | |
| (minutes) | | | | | |

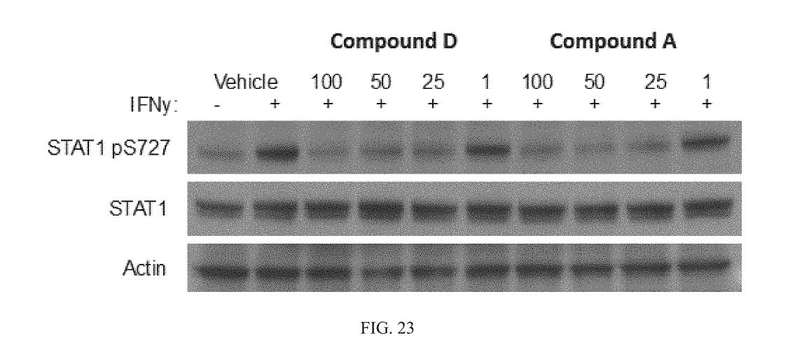

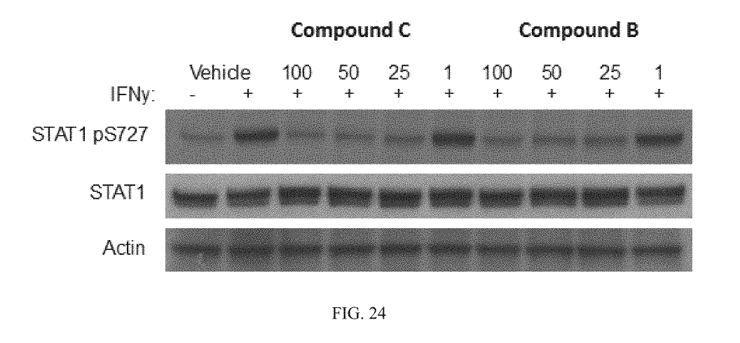

The dose response curves presented above were corroborated with western blot studies. FIG. 23 shows Compounds A and D and FIG. 24 shows Compounds B and C to have dose depended responses.

### Example 10: Half-Maximal Growth Inhibition (GI50) for Compounds a, B, C, D, and F Against Various Human Cancer Cell Lines

Compounds A, B, C, D, and F were tested against various cell lines to probe their broad utility against cancer. Cortistatin A (CA) was also tested against the cell lines in a head to head fashion. The data (below) shows that all 5 CA analogs tested had essentially the same sensitivity profile as Cortistatin A.

TABLE 14
GI₅₀ values of Compounds A, B, C, D, and F against cancerous cell lines

| Cell | | Selected | GI₅₀ (nM), day 10 | | | | | |
|---|---|---|---|---|---|---|---|---|
| line | Malignancy | Alteration | CA | F | A | D | C | B |
| SKNO-1 | AML, M2 | RUNX1-RUNX1T1 | <1 | <1 | 2 | <1 | 3 | 3 |
| MV4; 11 | AML, M5 | MLL-AF4 | <1 | 1 | 1.5 | 1.5 | 2 | 4 |
| MOLM-14 | AML, M5 | MLL-AF9 | <1 | 3 | 6 | 6 | 21 | 31 |
| HEL | AML, | JAK2V617F | >1,000 | ND | >1,000 | >1,000 | >1,000 | >1,000 |
| | erythroleukemia | | | | | | | |
| MKPL1 | AML, M7 | | <1 | ND | ND | 1.5 | ND | 2 |
| OCI-AML2 | AML, M4 | DNMT3A | 1 | ND | 4 | ND | ND | ND |
| | | R635W | | | | | | |
| K562 | CML | BCR-Abl | >1,000 | ND | ND | >1,000 | ND | ND |

### Example 11: Compounds A, B, C, and D CDK8 Mechanism of Action Validation

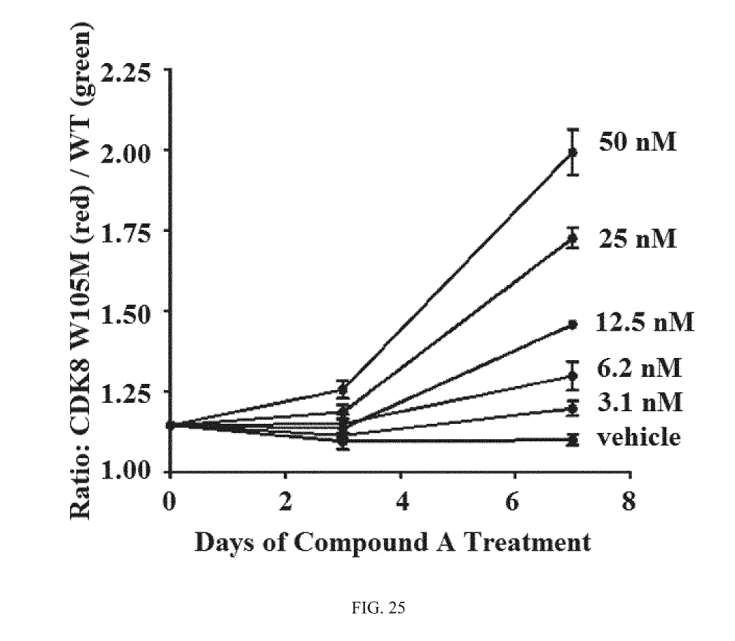

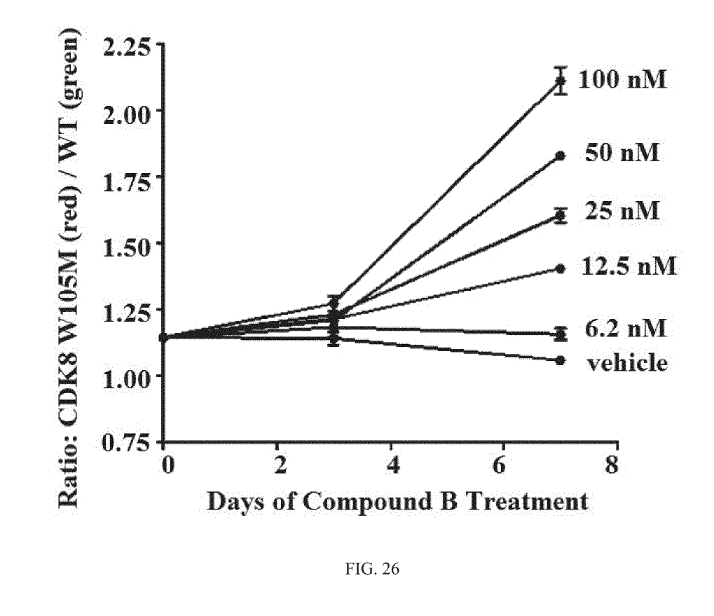

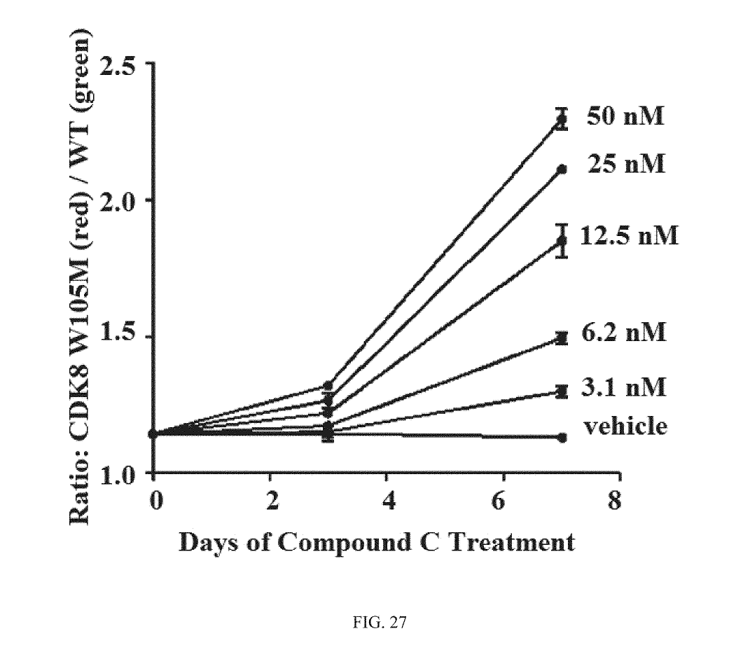

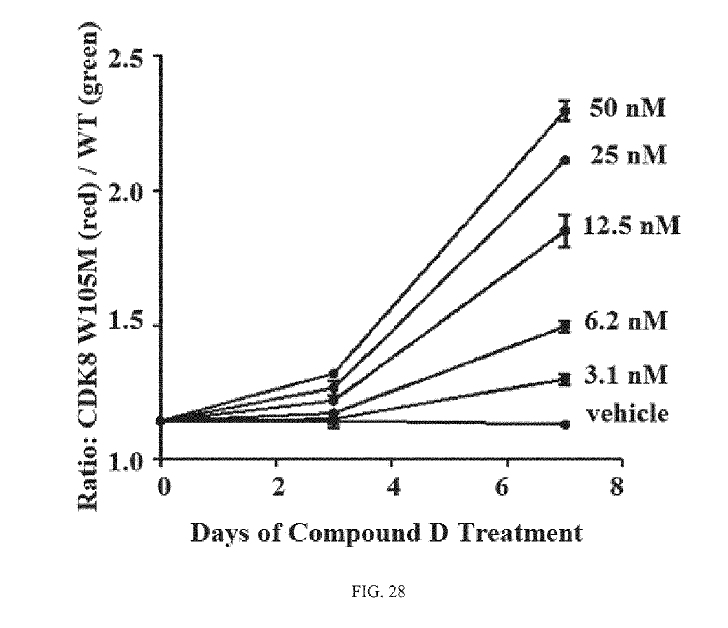

Compounds A, B, C, and D inhibit SET-2 AML cell line proliferation by inhibiting CDK8. ZsGreen (Clontech) and FLAG-CDK8 WT (wild type) or mCherry (Clontech) and FLAG-CDK8-W105M were expressed in SET 2 cells. The cells were mixed, the ratio of red to green fluorescence was determined by flow cytometry, and the cells were treated with the indicated compounds. After 3 days and 7 days of treatment, the ratio of red to green fluorescent cells was determined. Analogs dose-dependently shifted the ratio of red to green cells, indicating that CDK8 mediates the antiproliferative activity of Compounds A, B, C, and D as seen in FIGS. 25, 26, 27, and 28 respectively.

### Example 12: Pharmacokinetic Profile of Compounds A, B, C, and D

The objective of this study was to evaluate the pharmacokinetic (PK) profile of Compounds A, B, C, and D. On the day of dose administration the dosing solutions for each group were prepared by addition of vehicle [20% 2-Hydroxypropyl-beta-cyclodextrin (HPCD)]. Formulation correction for salt content was not required for this study. Materials were vortexed and sonicated at room temperature following addition of vehicle. The solutions were maintained at ambient temperature stirring in the dark for at least 30 minutes prior to dose administration. The formulations were then vortexed and sonicated briefly to help ensure solubility. These procedures resulted in clear colorless solutions.

Mice were selected from the Testing Facility's rodent colony. The animals were enrolled in the study based on acceptable health as determined by Testing Facility personnel on the day of test article administration. Animals were held in environmental acclimation for at least 48 hours (±6 hours) prior to dosing. Animals were maintained at temperatures between 16-26° C. (62-78° F.) with the humidity range between 30-70%. Animals in all groups were fasted overnight and food was returned 4 hours following dosing. Water was provided ad libitum throughout the study.

Whole blood samples for plasma were either collected from each animal by either direct vein puncture of the submandibular vein (survival blood collection) or by cardiac puncture following euthanasia with CO₂ (terminal blood collection). Throughout dosing and at all sample collection time points, all animals were observed for any clinically relevant abnormalities and none were observed. Following collection, whole blood was placed into a tube containing K₂EDTA and immediately placed on wet ice until processed for plasma. Whole blood samples were centrifuged at 2200×g for 10 minutes in a refrigerated centrifuge (5±3° C.) to isolate plasma. The samples were transferred to individual polypropylene containers and immediately placed on dry ice before storage at −70±10° C. until analysis. Plasma collected from undosed spare animals was pooled and analyzed as well.

Once collected the mouse plasma studies were quantitated using LCMS/MS spectroscopy to determine the concentration of compound. The below (Table 15) LC-MS/MS method was used to analyze samples and determine concentrations of the parent compound by the method of internal standards. Eight calibration standards containing the analyte and internal standard imipramine were prepared and analyzed at the beginning and at the end of the sequence. A calibration curve was generated and sample concentrations calculated using the Quantitation software MassHunter.

TABLE 15
LC-MS/MS Method used to determine PK Properties
Agilent 6460 Triple Quad LC/MS with Agilent 1290 Infinity HPLC

| | |
|---|---|
| MS Serial number | SG13227213 |
| Source | ES+ |
| DMRM mode | PAR (quant): 499 −> 378; Frag = 155 V, CE = 37 eV |
| | PAR (qual): 499 −> 156; Frag = 155 V, CE = 73 eV |
| | Int Std: 281 −> 208; Frag = 95 V, CE = 22 eV |
| Column | Gemini C18, 5 μM, 6 × 50 mm |
| MPA | 0.1% FA in water |
| MPB | 0.1% FA in water |
| Flow (mL/min) | 0.5 |
| Gradient | T = 0-2-6-8-8.1-10 |
| | % B = 0-0-100-100-0-0 |
| Injection volume | 5 μL |

The analyzed PK properties are presented in Table 16. Compounds A, B, C, and D were profiled for their pharmacokinetic properties and all exhibited good bioavailability, half-life, Cₘₐₓ, and appreciably less hERG activity than their unsubstituted analogs compounds E and F. Notably, the Tₘₐₓ varied from 0.5 hr (Compound A) to 8.0 hr (Compound C) suggesting that small modifications to the A ring can cause major changes in PK properties.

TABLE 16
PK Properties of Compounds A, D, C, and B

| | | Compound A | Compound D | Compound C | Compound B |
|---|---|---|---|---|---|
| | Mol. Weight | 413.55 | 498.6 | 467.66 | 481.67 |
| | (g/mol) | | | | |
| | cLogP | 5.29 | 4.53 | 4.68 | 4.94 |
| IV | Clearance | 18.4 | 18.0 | 23.7 | 31.0 |
| 3 mg/kg | (mL/min/kg) | | | | |
| CD-1 mice | Vss (L/kg) | 1.12 | 5.41 | 26.4 | 26.6 |
| | ½ life (hours) | 4.08 | 6.60 | 14.7 | 18.7 |
| PO | % F | 43.9% | 15.0% | 42.3% | 19.8% |
| 10 mg/kg | Cₘₐₓ (nM) | 3.869 | 377 | 462 | 218 |
| CD-1 mice | Tₘₐₓ (hours) | 0.5 | 2.0 | 8.0 | 2.0 |
| | ½ life (hours) | 3.04 | 4.25 | 6.68 | 9.55 |
| | AUC_{inf} | 3,970 | 1,390 | 2,970 | 1,060 |
| | (ng*h/mL) | | | | |
| | C24 (nM) | 1.8 | 7.6 | 82.5 | 26.4 |
| | hERG inhibition | >30 | 6.3 | 10.9 | 10.7 |
| | (μM) | | | | |

### Example 13: In Vivo Efficacy of Compounds a, B, C, D, and F

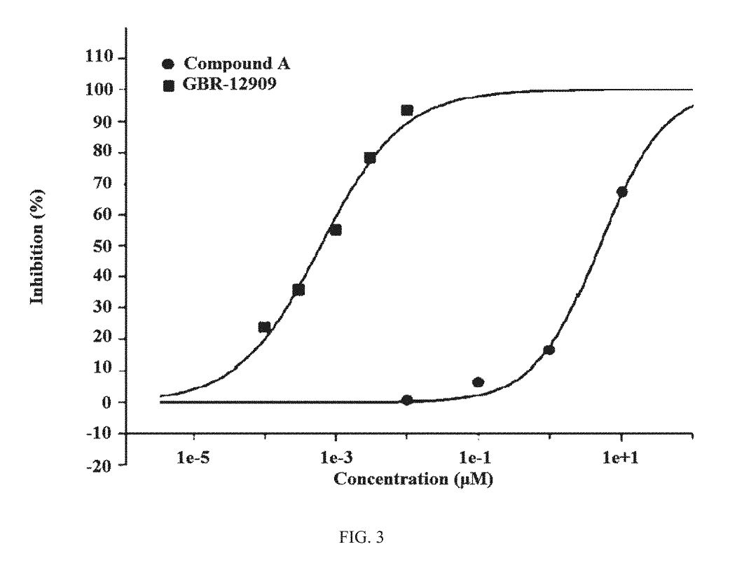

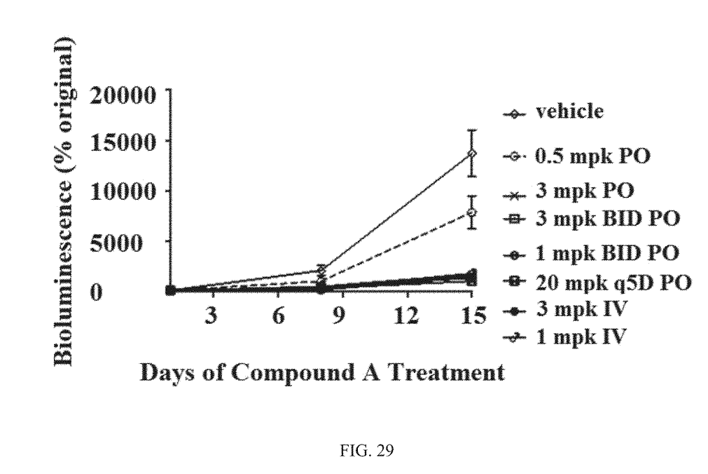

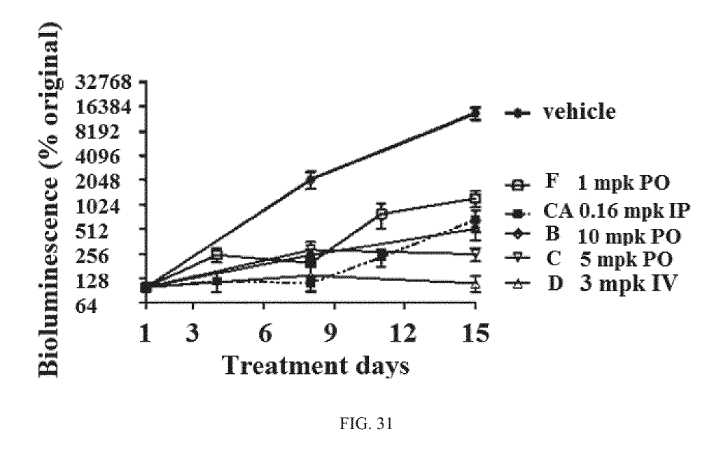

The in vivo anti-leukemic activity of Compounds A, B, C, D, and F was tested at the indicated doses and treatment schedules. The doses were chosen based on the compounds tolerability profile (i.e. CA was dosed at 0.16 mg/kg because higher doses caused weight loss or other tolerability issues outlined in Example 6). Human AML cell line MV4;11 expressing mCherry and luciferase were injected into NSG mice. Upon measurement of leukemia cell engraftment, treatment was initiated. Body weight and bioluminescence were measured. It has previously been shown (Shair, Nature 2015) that bioluminescence corresponds to leukemia burden. FIG. 29 shows that Compou0.5nd A was highly efficacious at doses of at least 1 mg/kg and even showed some efficacy at 0.5 3.04 mg/kg. Moreover there was no appreciable advantage between IV or PO dosing observed. FIG. 3, 970 30 (log 2 scale) shows that Compound A is similarly efficacious to Compound F in addition to 1.8 its superior selectivity and tolerability profile. Compound F was dosed at 1 mg/kg because high>30er doses were found to cause tolerability issues. FIG. 31 (log 2 scale) shows that Compounds B, C, and D exhibit superior in vivo efficacy as compared to Compound F. All compounds inhibited leukemia progression.

### Example 14: Comparison of Compounds A, B, C, D, E, F, and CA

Compounds A, B, C, and D have reduced inhibition of the hERG ion channel compared to Compounds E and F. In addition, Compounds A, B, C, and D have a reduced number of off-target interactions as measured by the number of targets inhibited by >70% dosed at 10 μM against 118 off-targets (Panlabs). These results indicate that Compounds A, B, C, and D have an improved safety profile compared to Compounds E and F.

Compounds A, B, C, and D also are able to achieve significantly higher exposure (AUC) in mice than previous compounds including CA and Compound F. The improved safety profile of the new analogs may allow for greater exposure achievable at well-tolerated doses. Compounds B, C, and D exhibit better efficacy in a mouse model of AML than CA and Compound F, with Compound A having comparable efficacy. The greater efficacy of these compounds correlates largely with the increased exposure achievable at doses that are well-tolerated. The higher exposure achievable with these new analogs may be translating into greater efficacy and/or the better safety profiles are enabling higher exposure and thus improved efficacy in vivo. In other words, off-target interactions of CA and Compound F limit exposure and efficacy. These off-target interactions have been mitigated with the new compounds presented in the present invention, resulting in greater exposure and in vivo efficacy.

TABLE 17
In vivo Efficacy of Compounds A, B, C, D, E, and F

| In vivo efficacy | | | | | | Safety Profile |
|---|---|---|---|---|---|---|
| | | Fold increase | | | | 3 hits ≥75% |
| | | in leukemia | Efficacy | AUC_{inf} | hERG | inhibition |
| | | burden 15 days | Rank | (ng* | (IC₅₀, | at 10 μM |
| Compound | Dose/Schedule | after 1^{st} dose | Day 15 | hr/mL) | μM) | out of 28 |
| D | 3 mpk IV 7on/2off/4on then | 1.1 | 1 | 2780 | 6.282 | 3 |
| | IP 2on* | | | | | |
| C | 5 mpk PO 7on/4off/4on | 2.2 | 2 | 1485 | 10.932 | 4 |
| B | 10 mpk PO 7 on/2off/6on | 5.2 | 3 | 1060 | 10.732 | 8 |
| Cortistatin | 0.16 mpk IP QDx15 | 6.7 | 4 | 134 | ND | ND |
| A | | | | | | |
| A | 20 mpk PO Q5Dx2 | 10.4 | 5 | 7940 | >30** | 0 |
| F | 1 mpk PO QDx15 | 12.3 | 6 | 524 | 0.37 | 12 |
| E | ND | ND | NA | ND | 0.562 | ND |
| vehicle | PO QDx15 | 126 | NA | | | |

Dose/Schedule: QD = once daily (QDx15 = once daily for 15 doses); on/off = daily treatment or holiday (QD 7on/2off = once daily for 7 days on/2 days off; PO = oral, IP = intraperitoneal, IV = intravenous
In vivo efficacy = MV4; 11 disseminated leukemia model in NSG mice. Data from 3 separate studies: (1) cortistatin A, (2) Compound F and (3) Compounds A, B, C, and D. Vehicle shown for study 3. Vehicle for cortistatin A and Compound F studies had similar fold-change (118 and 122, respectively).
AUCinf = area under the curve at the indicated efficacy dose. Values are actual of interpolated from pharmacokinetic study performed in male CD-1 mice
Safety Panel = 28 binding assays (Eurofins Panlabs)
In vivo efficacy = MV4; 11 disseminated leukemia model in NSG mice
*Compound D IV switched to IP after veins were difficult to locate on mice
**some insolubility observed at 10 μM and 30 μM, only 9% inhibition at 3 μM.

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

What is claimed is:

1. A compound selected from: 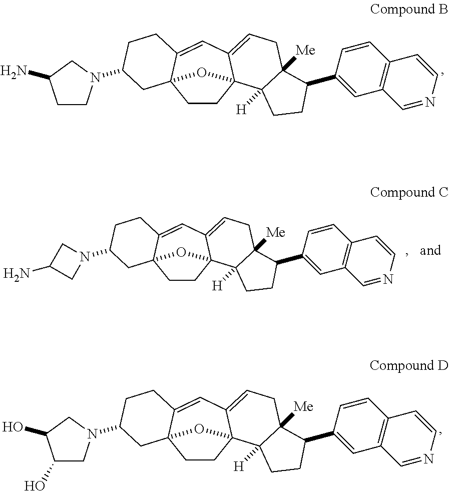 [SMILES: C[C@]12CC=C3C=C4CC[C@@H](N5CC[C@@H](N)C5)C[C@]45CC[C@]3(O5)[C@@H]1CC[C@@H]2c1ccc2ccncc2c1 ; C[C@]12CC=C3C=C4CC[C@@H](N5CC(N)C5)C[C@]45CC[C@]3(O5)[C@@H]1CC[C@@H]2c1ccc2ccncc2c1 ; C[C@]12CC=C3C=C4CC[C@@H](N5C[C@H](O)[C@@H](O)C5)C[C@]45CC[C@]3(O5)[C@@H]1CC[C@@H]2c1ccc2ccncc2c1]
or a pharmaceutically acceptable salt thereof.

2. The compound of claim 1, wherein the compound is 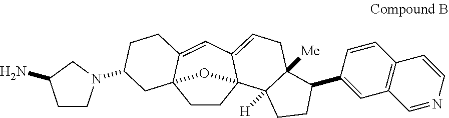 [SMILES: C[C@]12CC=C3C=C4CC[C@@H](N5CC[C@@H](N)C5)C[C@]45CC[C@]3(O5)[C@@H]1CC[C@@H]2c1ccc2ccncc2c1]
or a pharmaceutically acceptable salt thereof.

3. The compound of claim 1, wherein the compound is 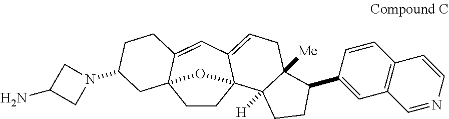 [SMILES: C[C@]12CC=C3C=C4CC[C@@H](N5CC(N)C5)C[C@]45CC[C@]3(O5)[C@@H]1CC[C@@H]2c1ccc2ccncc2c1]
or a pharmaceutically acceptable salt thereof.

4. The compound of claim 1, wherein the compound is 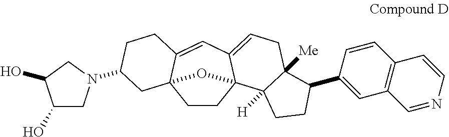 [SMILES: C[C@]12CC=C3C=C4CC[C@@H](N5C[C@H](O)[C@@H](O)C5)C[C@]45CC[C@]3(O5)[C@@H]1CC[C@@H]2c1ccc2ccncc2c1]
or a pharmaceutically acceptable salt thereof.

5. A compound selected from: 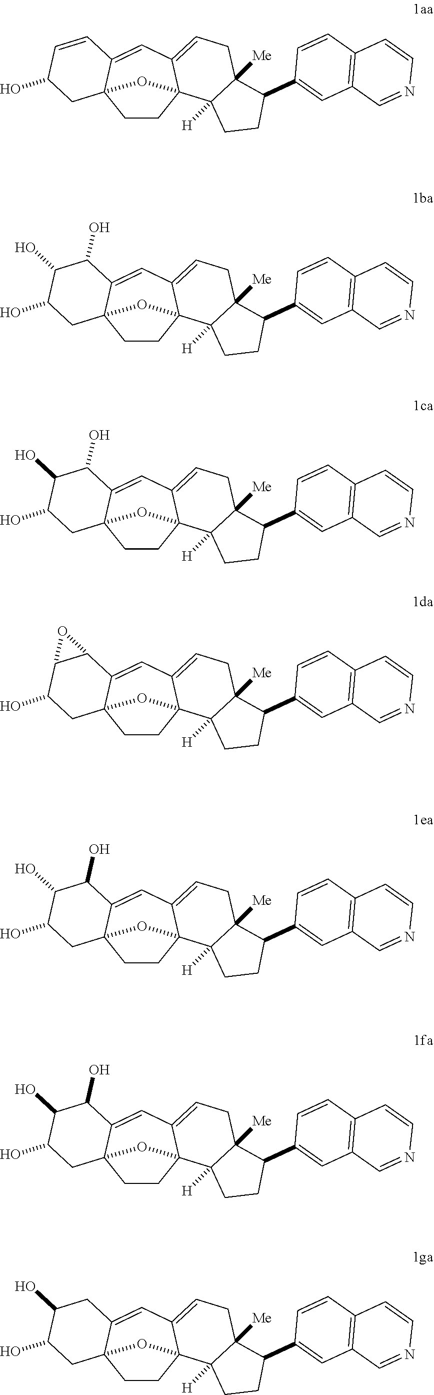 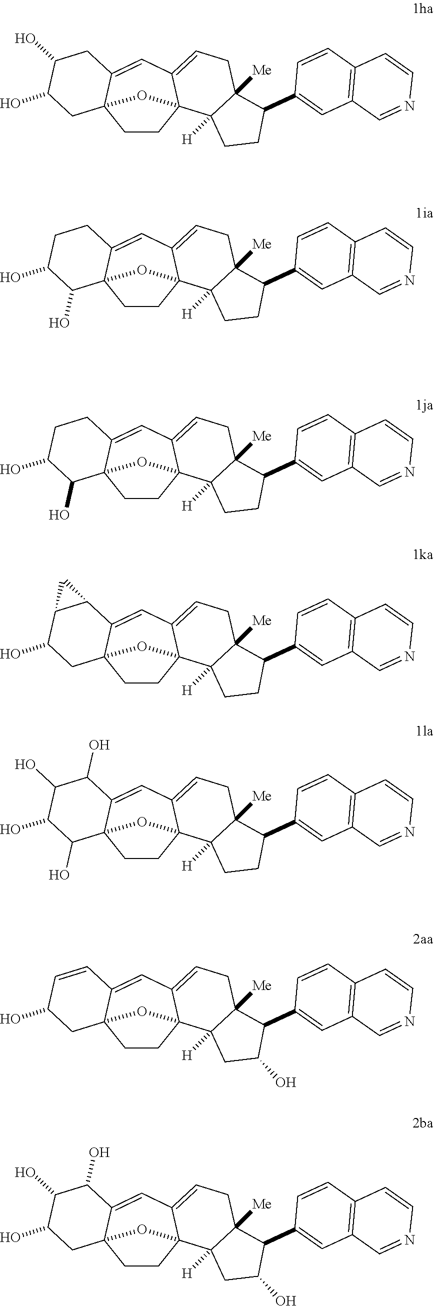 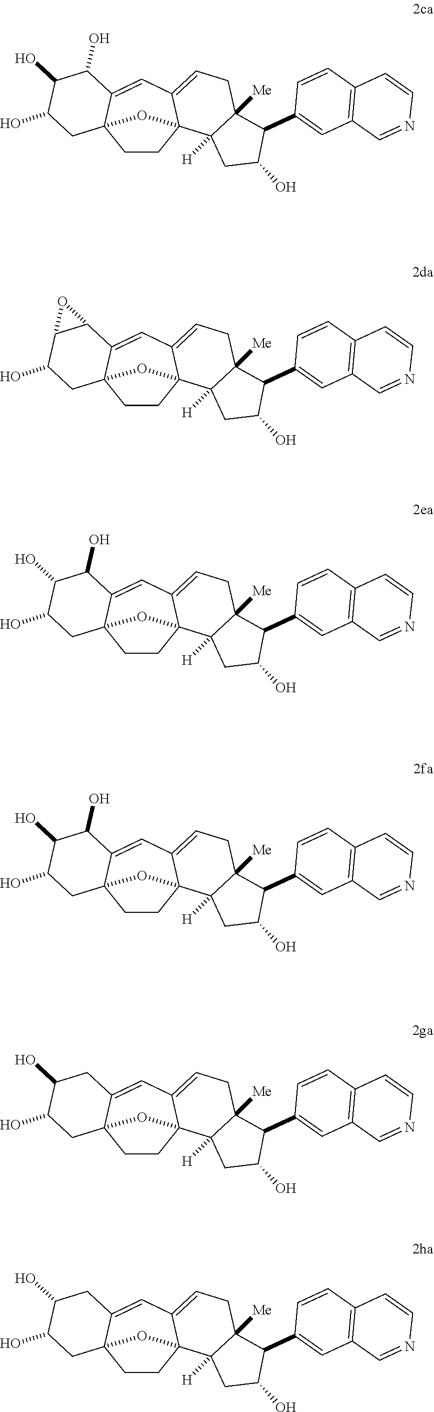 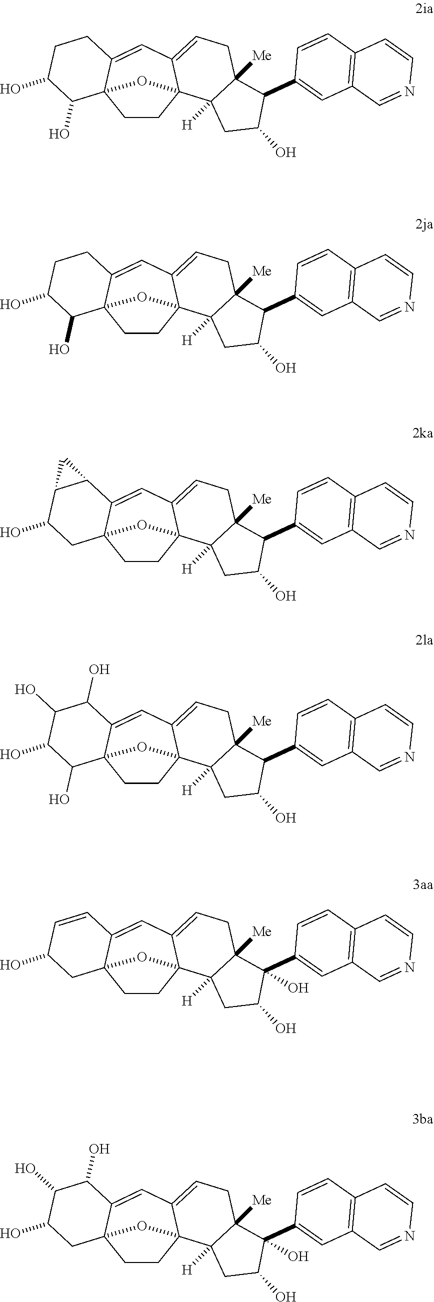 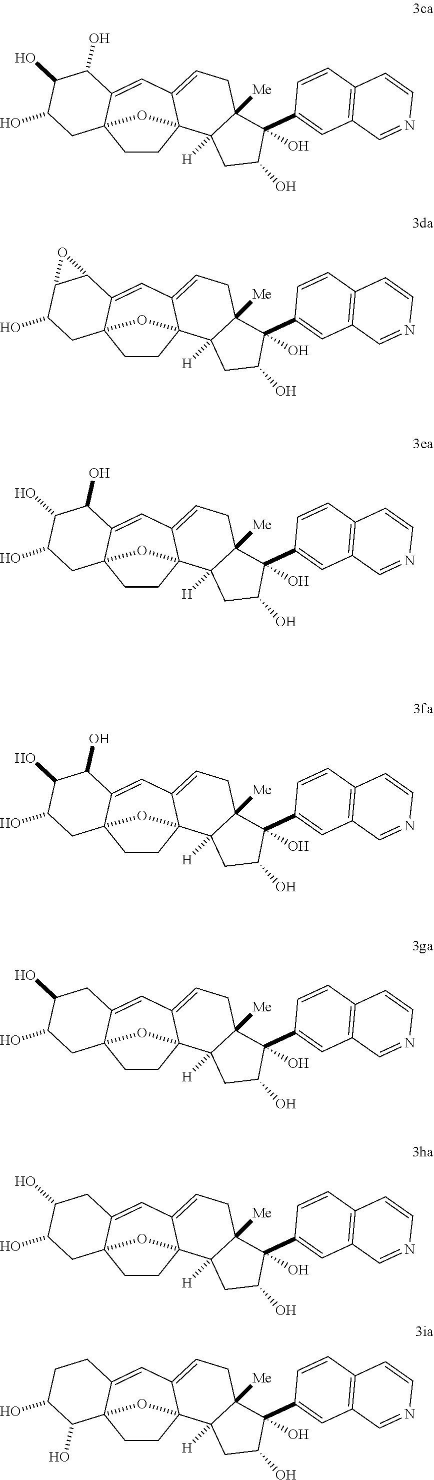 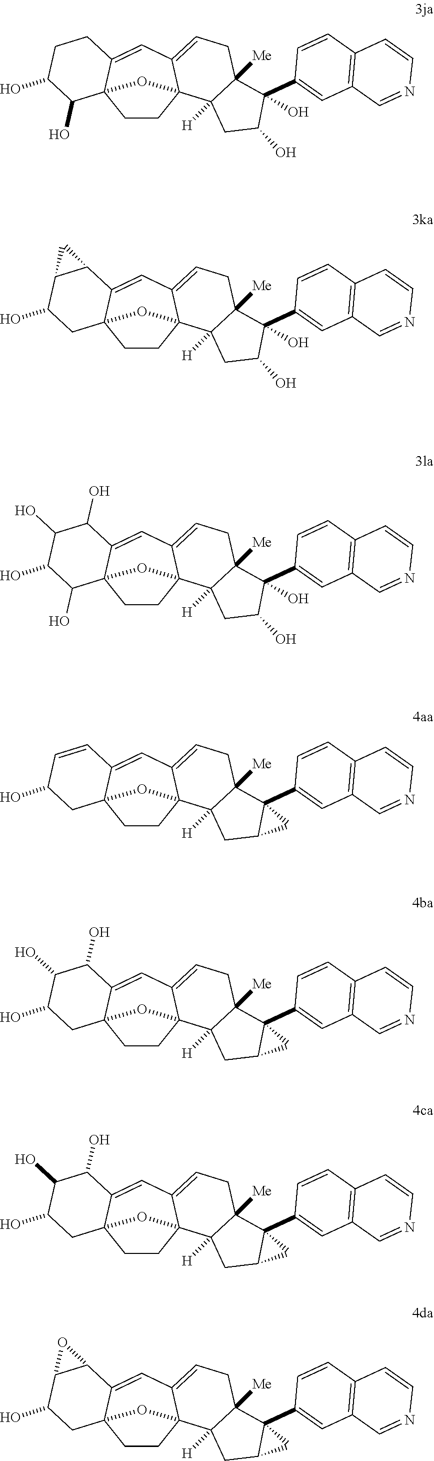 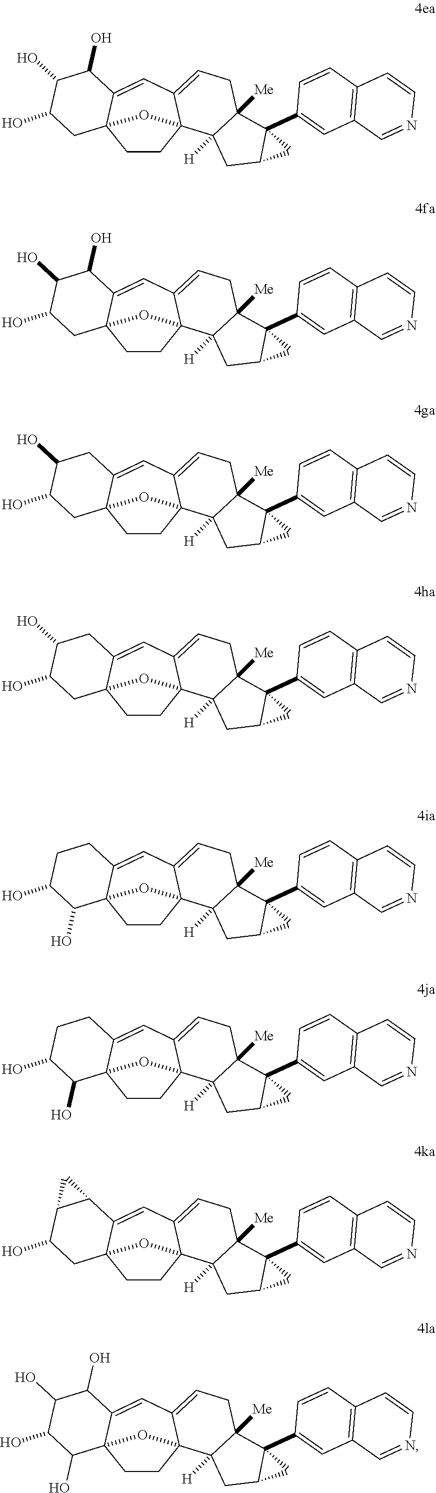
or a pharmaceutically acceptable salt thereof.

6. A compound selected from: 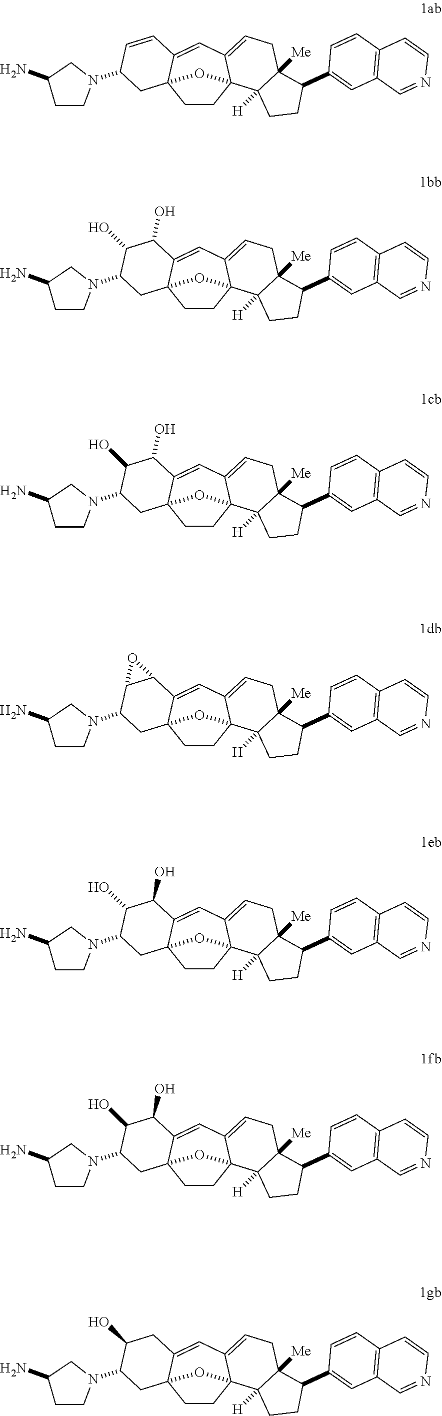 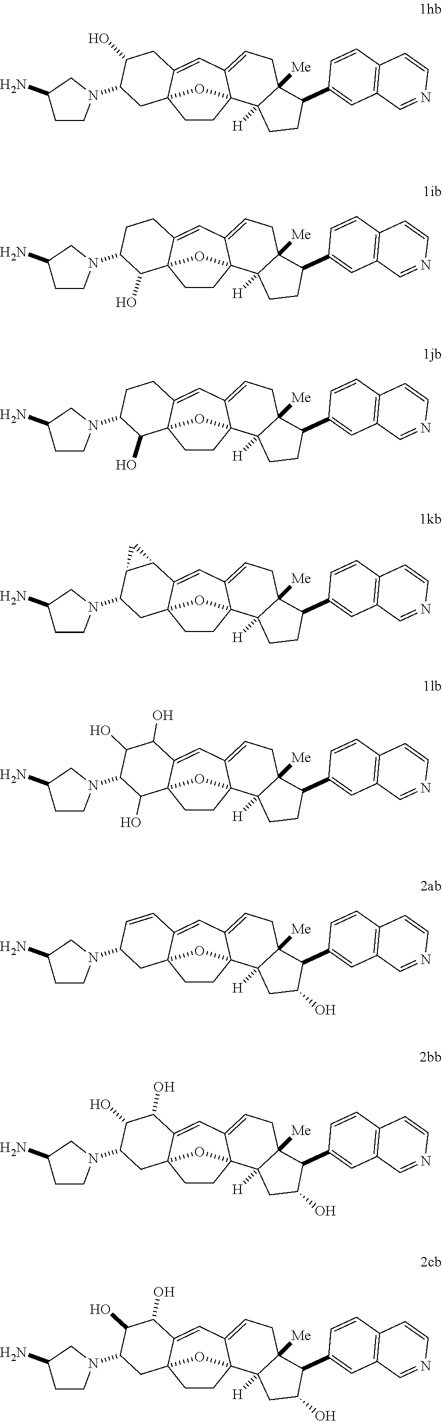 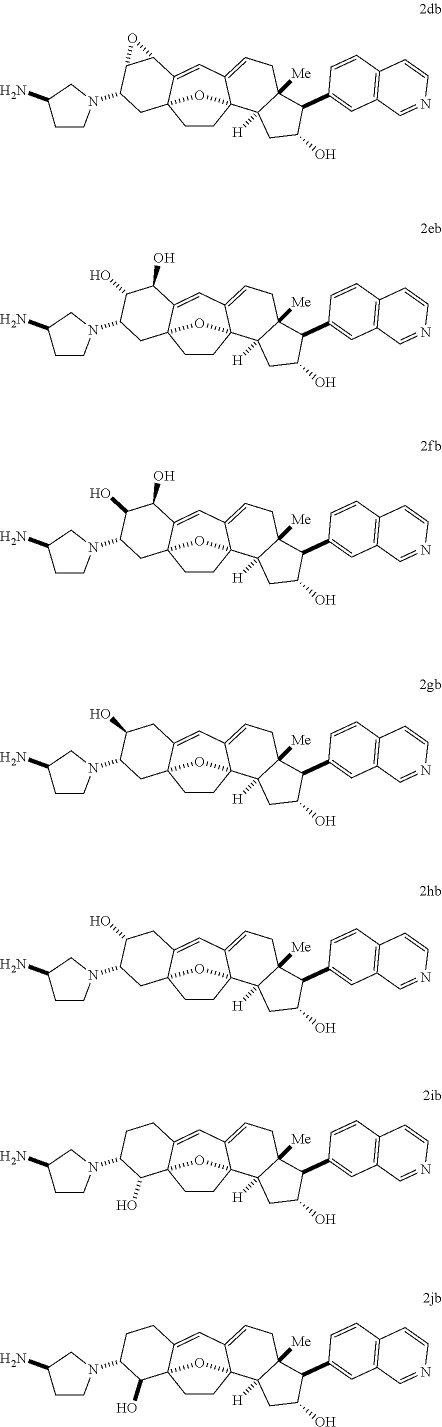 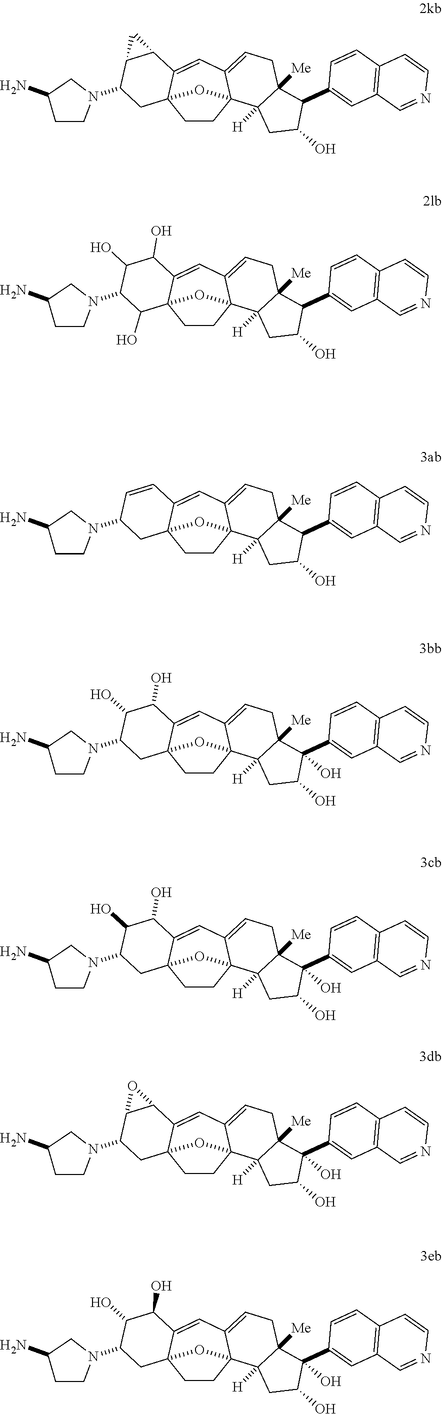 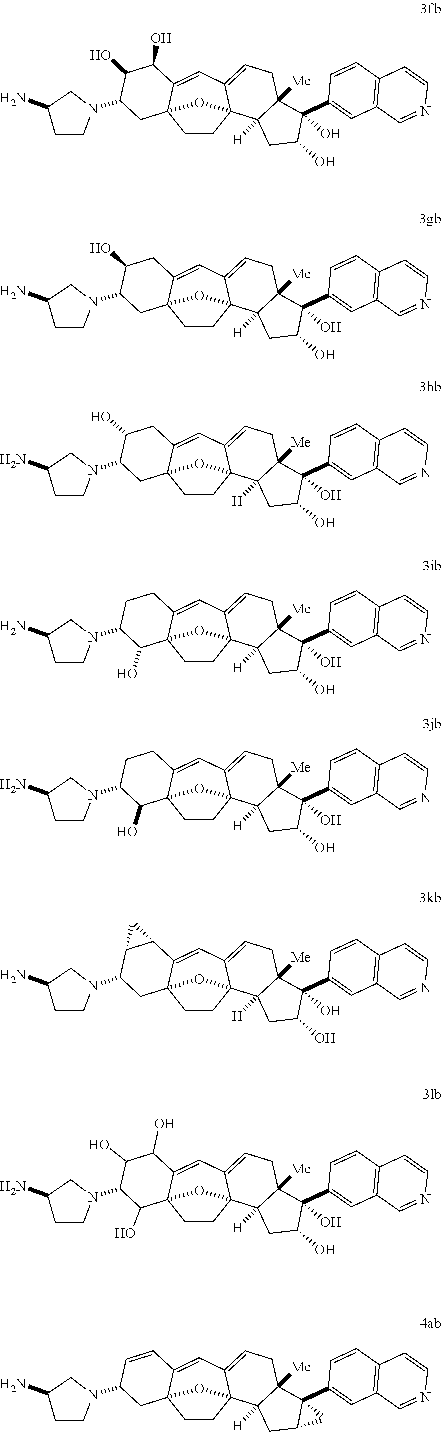 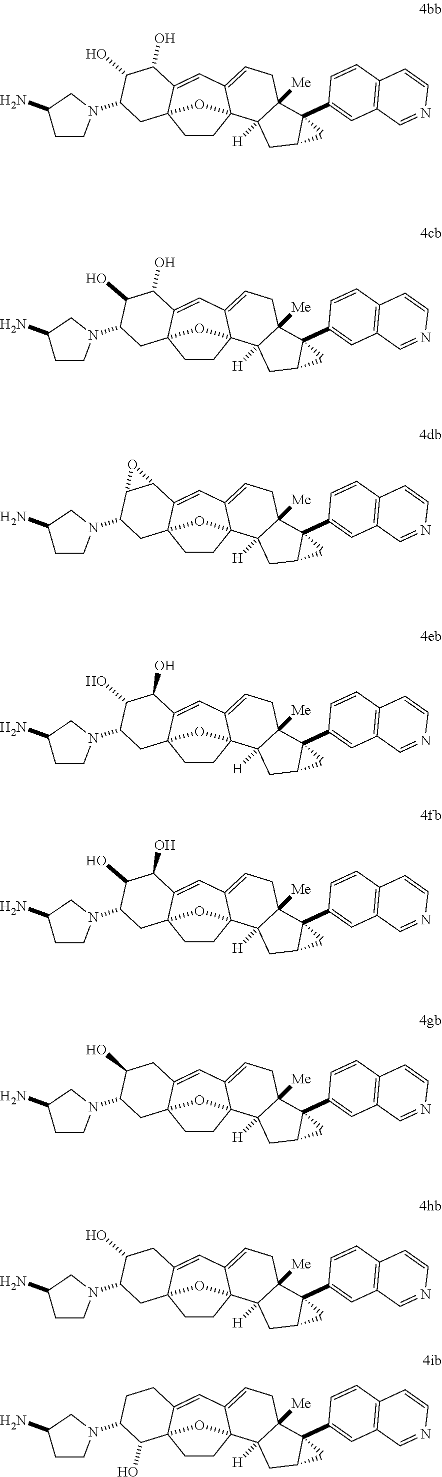 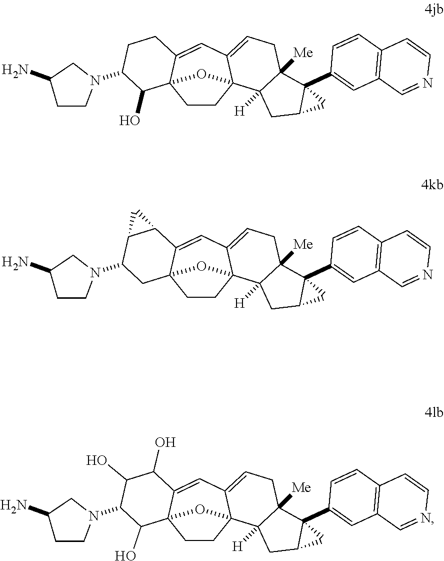
or a pharmaceutically acceptable salt thereof.

7. A compound selected from: 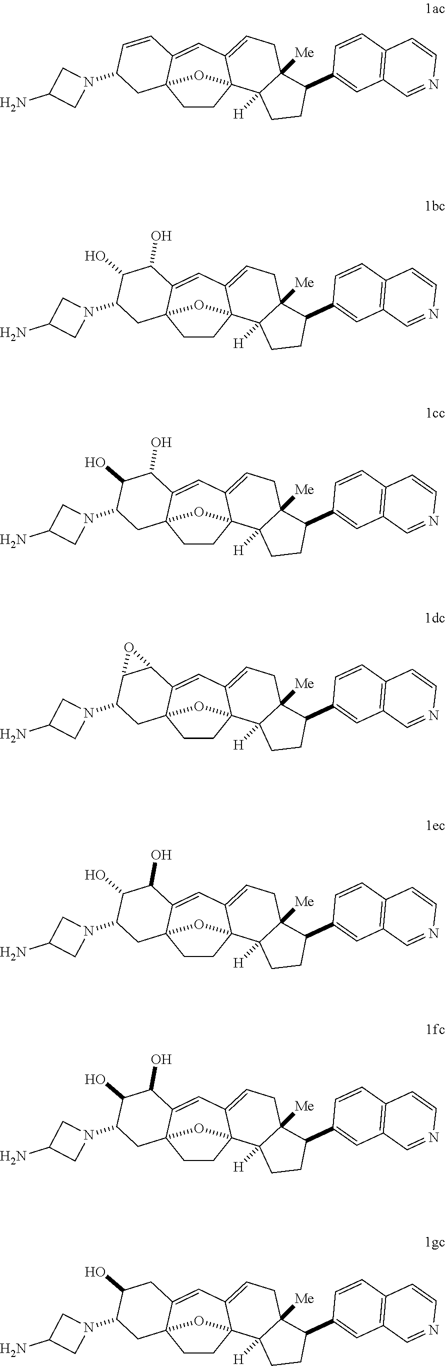 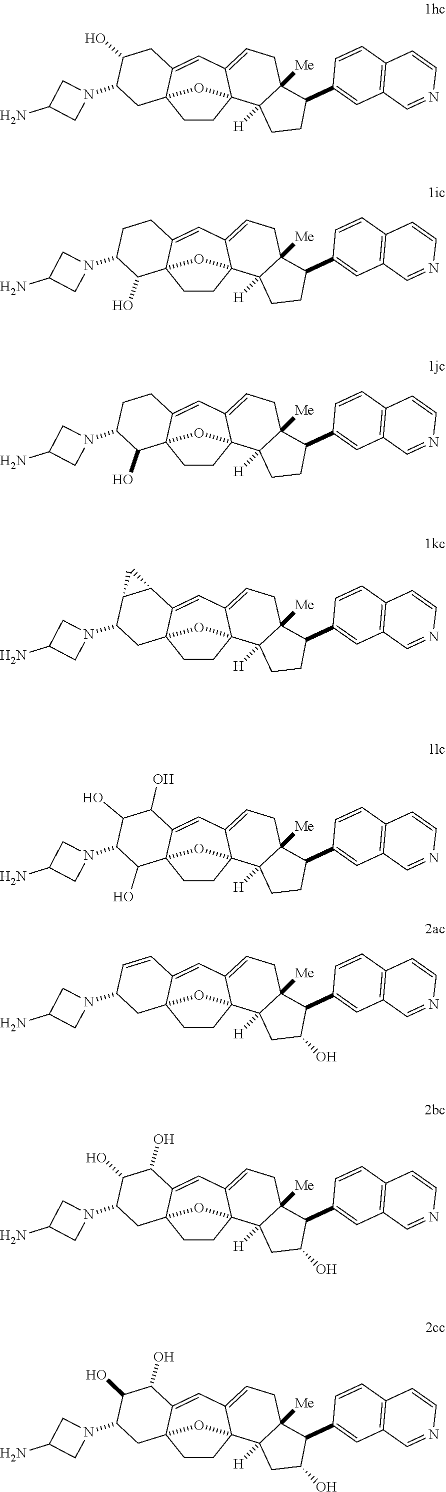 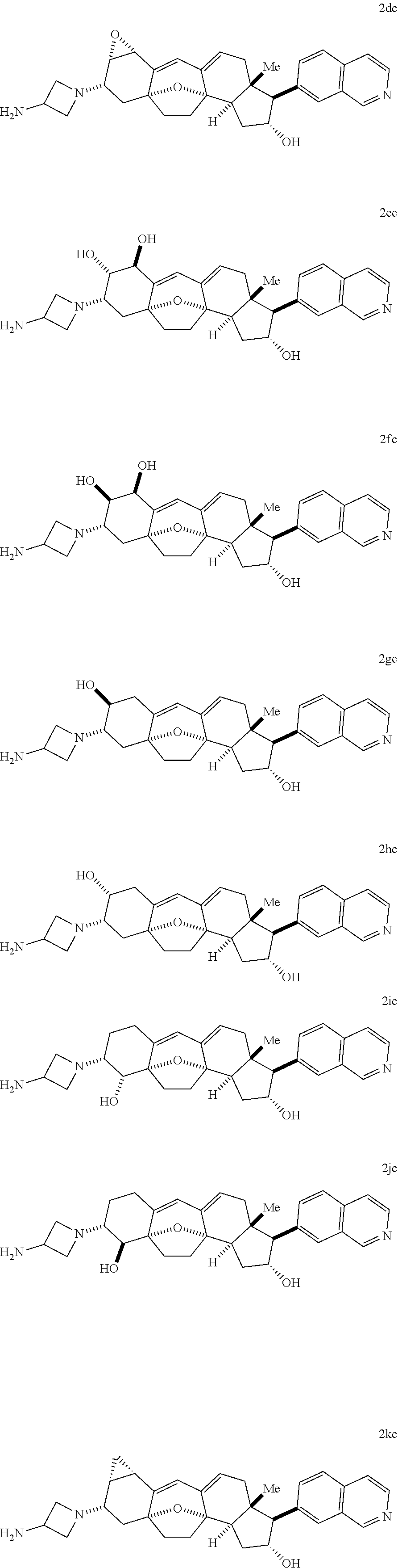 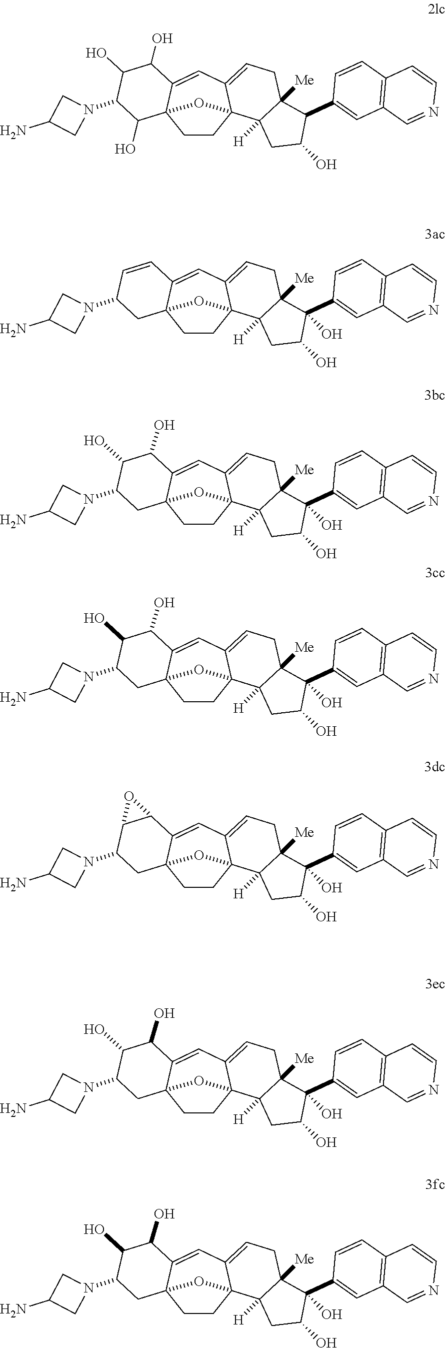 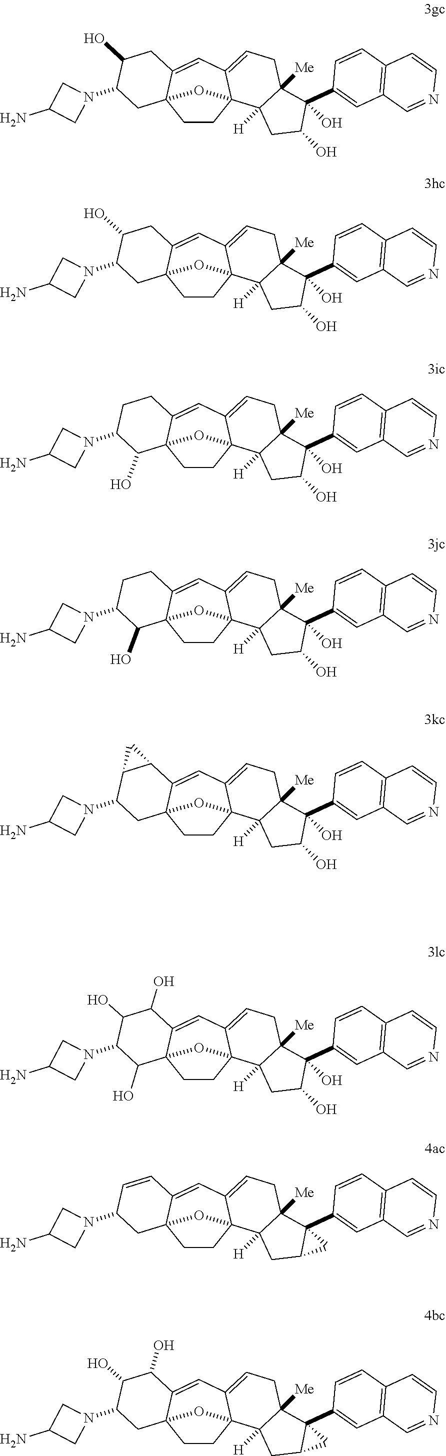 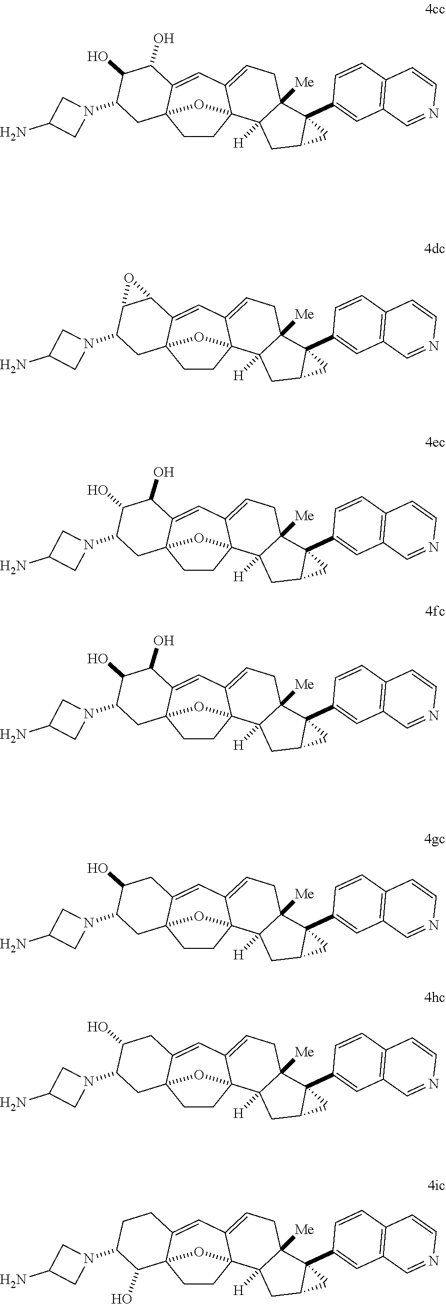 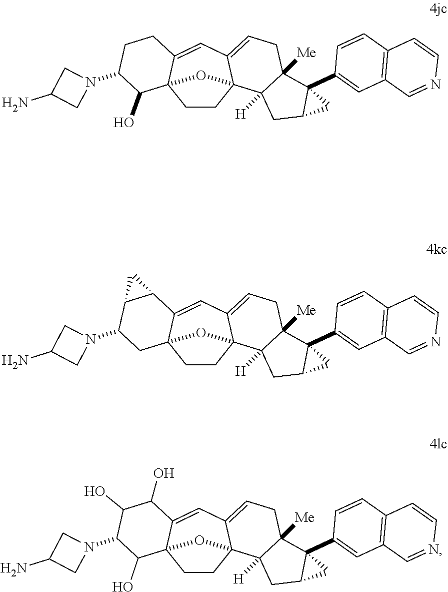
or a pharmaceutically acceptable salt thereof.

8. A compound selected from: 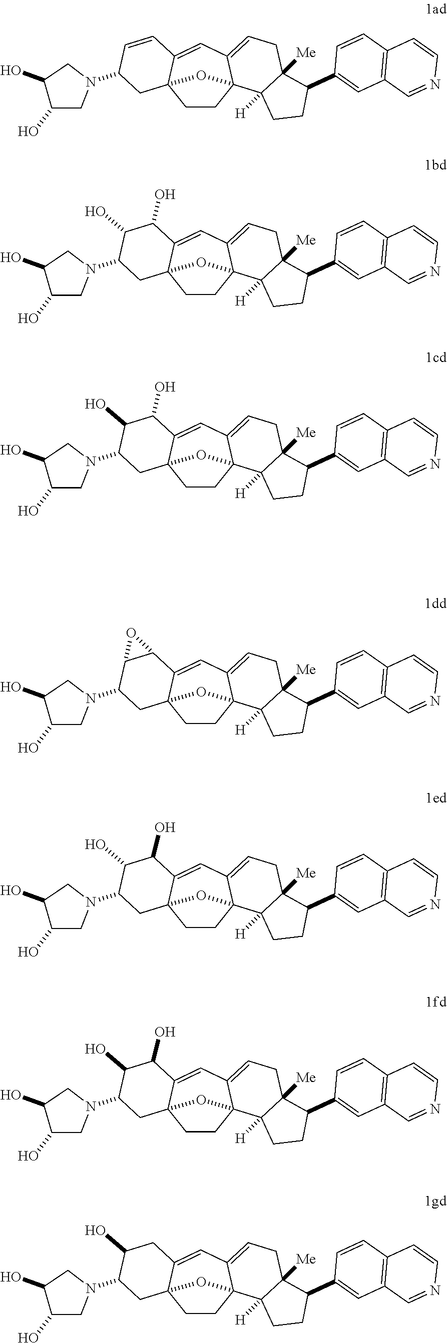 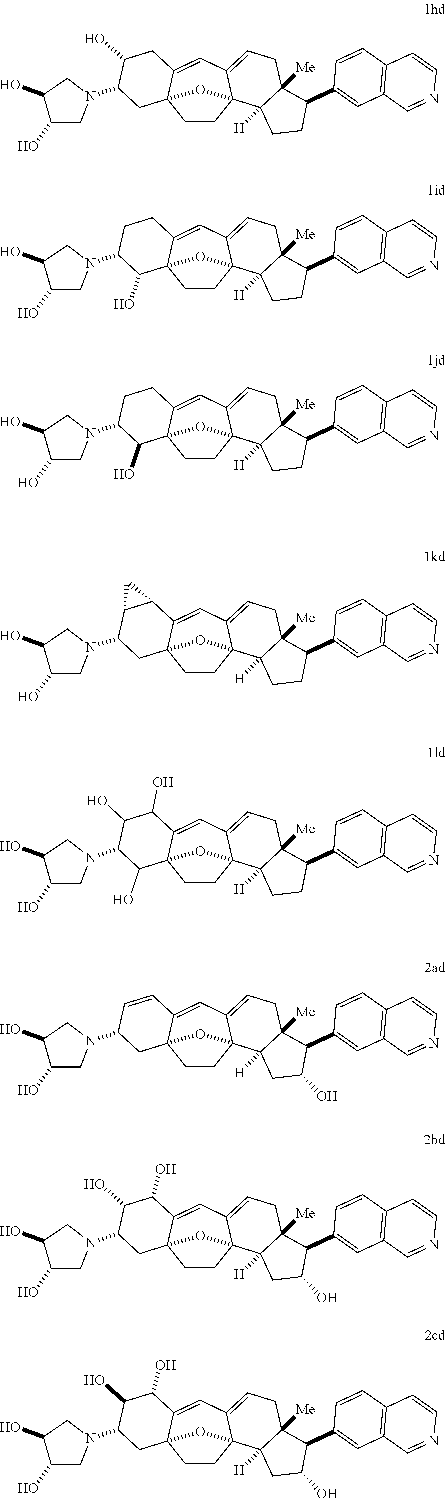 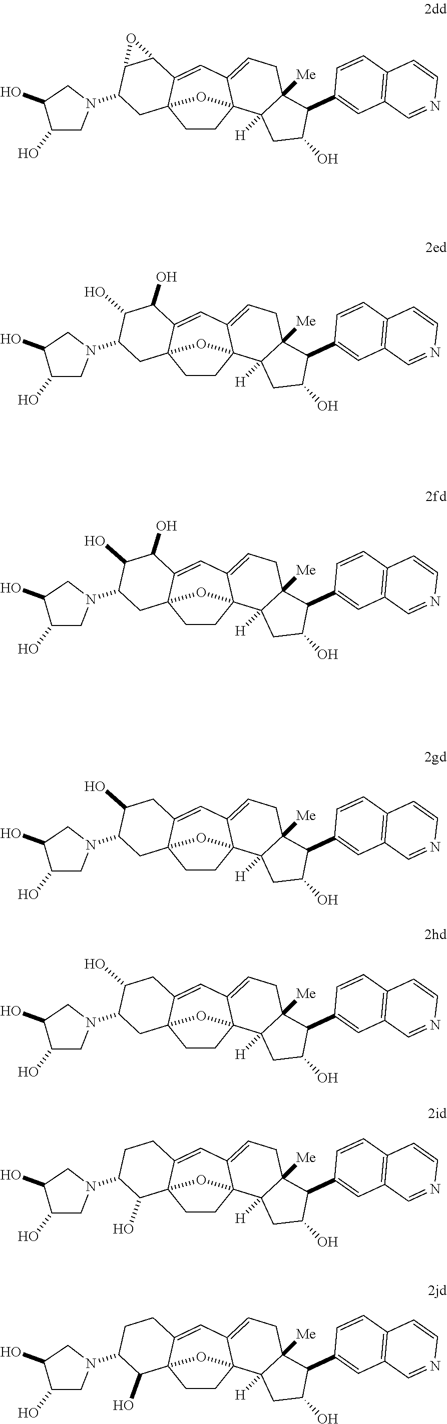 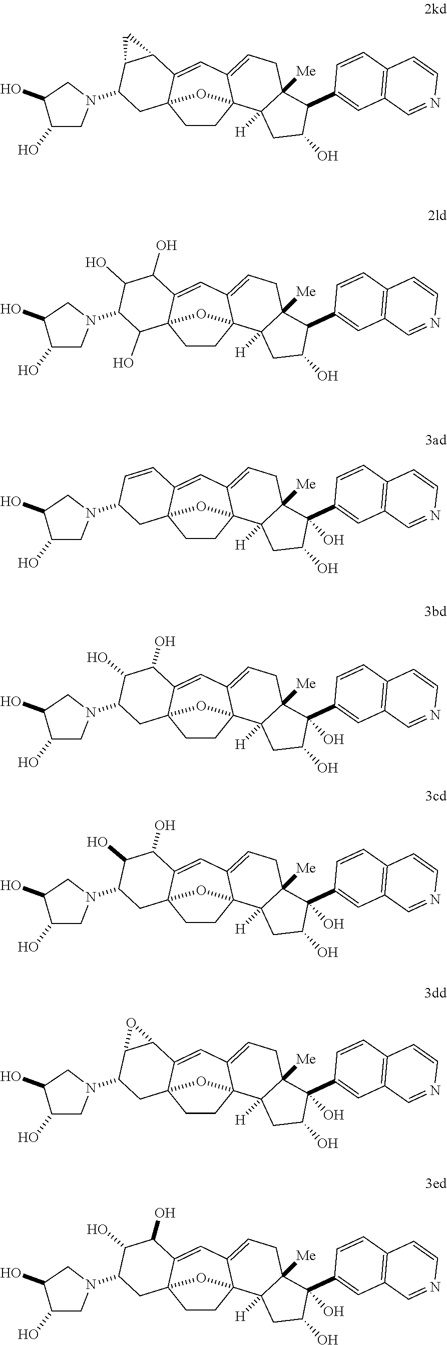 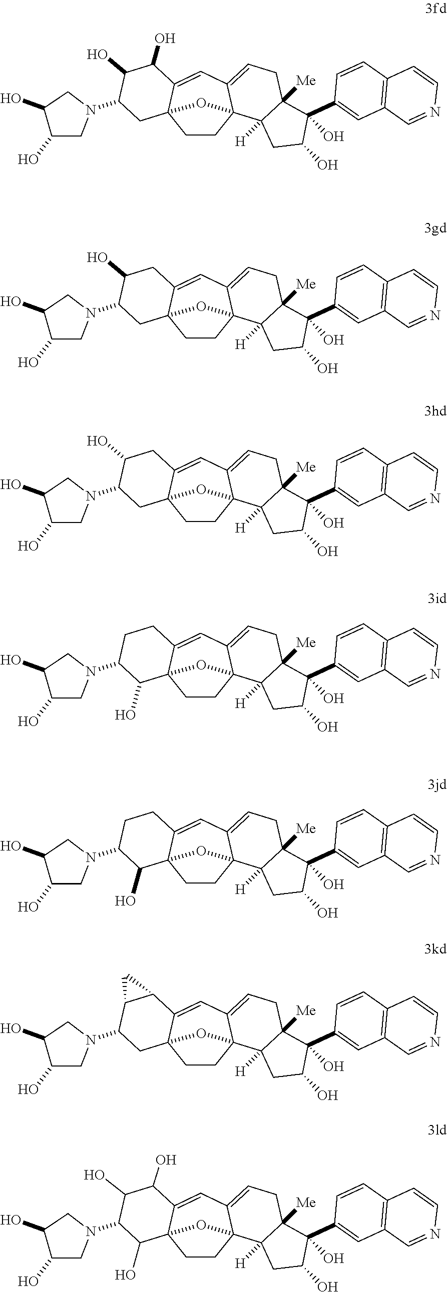 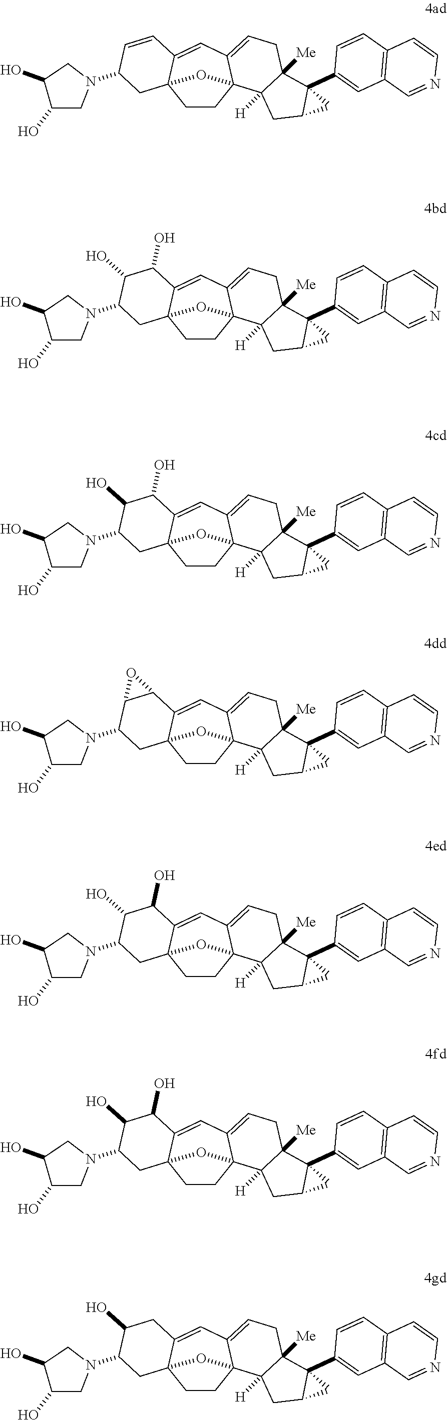 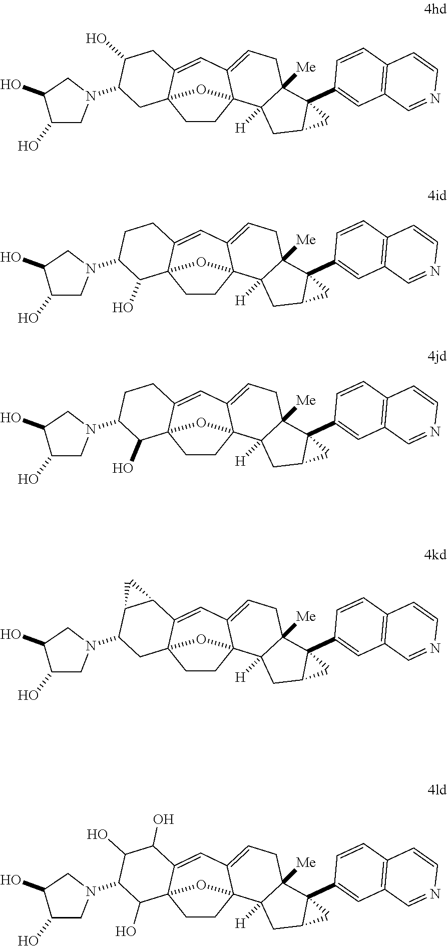
or a pharmaceutically acceptable salt thereof.

9. The compound of claim 1, wherein the compound is not in a salt form.

10. The compound of claim 1, wherein at least one hydrogen is replaced with deuterium.

11. A method for the treatment of a host with a disorder mediated by CDK8 and/or CDK19 comprising administering to the host an effective amount of a compound selected from 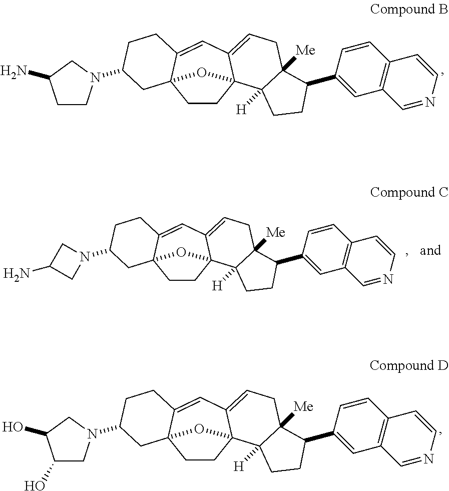 [SMILES: C[C@]12CC=C3C=C4CC[C@@H](N5CC[C@@H](N)C5)C[C@]45CC[C@]3(O5)[C@@H]1CC[C@@H]2c1ccc2ccncc2c1 ; C[C@]12CC=C3C=C4CC[C@@H](N5CC(N)C5)C[C@]45CC[C@]3(O5)[C@@H]1CC[C@@H]2c1ccc2ccncc2c1 ; C[C@]12CC=C3C=C4CC[C@@H](N5C[C@H](O)[C@@H](O)C5)C[C@]45CC[C@]3(O5)[C@@H]1CC[C@@H]2c1ccc2ccncc2c1]
or a pharmaceutically acceptable salt thereof.

12. The method of claim 11, wherein the host is a human.

13. The method of claim 12, wherein the compound is 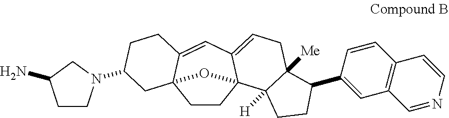 [SMILES: C[C@]12CC=C3C=C4CC[C@@H](N5CC[C@@H](N)C5)C[C@]45CC[C@]3(O5)[C@@H]1CC[C@@H]2c1ccc2ccncc2c1]
or a pharmaceutically acceptable salt thereof.

14. The method of claim 12, wherein the compound is 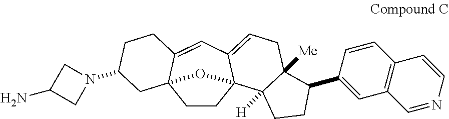 [SMILES: C[C@]12CC=C3C=C4CC[C@@H](N5CC(N)C5)C[C@]45CC[C@]3(O5)[C@@H]1CC[C@@H]2c1ccc2ccncc2c1]
or a pharmaceutically acceptable salt thereof.

15. The method of claim 12, wherein the compound is 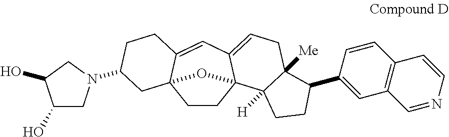 [SMILES: C[C@]12CC=C3C=C4CC[C@@H](N5C[C@H](O)[C@@H](O)C5)C[C@]45CC[C@]3(O5)[C@@H]1CC[C@@H]2c1ccc2ccncc2c1]
or a pharmaceutically acceptable salt thereof.

16. The method of claim 12, wherein the disorder is a tumor, a cancer, or a disorder related to abnormal proliferation.

17. The method of claim 12, wherein the disorder is an inflammatory disorder, an immune disorder, or an autoimmune disorder.

18. The method of claim 12, wherein the disorder is acute myeloid leukemia (AML).

19. The method of claim 12, wherein the disorder is lymphoma, myeloproliferative neoplasm (MPN), primary myelofibrosis (PMF), acute lymphocytic leukemia (ALL), chronic lymphocytic leukemia (CLL), or chronic myelocytic leukemia (CML).

20. A pharmaceutical composition comprising a pharmaceutically acceptable carrier and a compound selected from 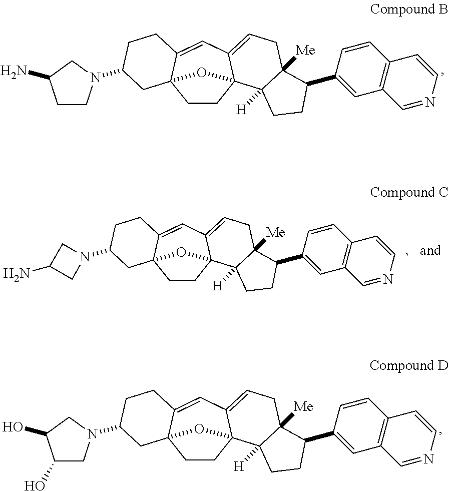 [SMILES: C[C@]12CC=C3C=C4CC[C@@H](N5CC[C@@H](N)C5)C[C@]45CC[C@]3(O5)[C@@H]1CC[C@@H]2c1ccc2ccncc2c1 ; C[C@]12CC=C3C=C4CC[C@@H](N5CC(N)C5)C[C@]45CC[C@]3(O5)[C@@H]1CC[C@@H]2c1ccc2ccncc2c1 ; C[C@]12CC=C3C=C4CC[C@@H](N5C[C@H](O)[C@@H](O)C5)C[C@]45CC[C@]3(O5)[C@@H]1CC[C@@H]2c1ccc2ccncc2c1]
or a pharmaceutically acceptable salt thereof.
